SEQUENCE LISTING (22 sequences)

SEQ ID NO: 1 - DNA, 1530 bp, synthetic construct
  misc_feature 1..1530: note = synthetic polymer
  source 1..1530: mol_type = other DNA
atgggagtgctaaaattcaagcacatctttttcagaagctttgttaaatcaagtggagtatcccagatagttttcaccttccttctgattccatgttgcttgactctgaatttcagagcacctcctgttattccaaatgtgcctttcctctgggcctggaatgccccaagtgaattttgtcttggaaaatttgatgagccactagatatgagcctcttctctttcataggaagcccccgaataaacgccaccgggcaaggtgttacaatattttatgttgatagacttggctactatccttacatagattcaatcacaggagtaactgtgaatggaggaatcccccagaagatttccttacaagaccatctggacaaagctaagaaagacattacattttatatgccagtagacaatttgggaatggctgttattgactgggaagaatggagacccacttgggcaagaaactggaaacctaaagatgtttacaagaataggtctattgaattggttcagcaacaaaatgtacaacttagtctcacagaggccactgagaaagcaaaacaagaatttgaaaaggcagggaaggatttcctggtagagactataaaattgggaaaattacttcggccaaatcacttgtggggttattatctttttccggattgttacaaccatcactataagaaacccggttacaatggaagttgcttcaatgtagaaataaaaagaaatgatgatctcagctggttgtggaatgaaagcactgctctttacccatccatttatttgaacactcagcagtctcctgtagctgctacactctatgtgcgcaatcgagttcgggaagccatcagagtttccaaaatacctgatgcaaaaagtccacttccggtttttgcatatacccgcatagtttttactgatcaagttttgaaattcctttctcaagatgaacttgtgtatacatttggcgaaactgttgctctgggtgcttctggaattgtaatatggggaaccctcagtataatgcgaagtatgaaatcttgcttgctcctagacaattacatggagactatactgaatccttacataatcaacgtcacactagcagccaaaatgtgtagccaagtgctttgccaggagcaaggagtgtgtataaggaaaaactggaattcaagtgactatcttcacctcaacccagataattttgctattcaacttgagaaaggtggaaagttcacagtacgtggaaaaccgacacttgaagacctggagcaattttctgaaaaattttattgcagctgttatagcaccttgagttgtaaggagaaagctgatgtaaaagacactgatgctgttgatgtgtgtattgctgatggtgtctgtatagatgcttttctaaaacctcccatggagacagaagaacctcaaattttctacaatgcttcaccctccacactatctgccacaatgttcattgttagtattttgtttcttatcatttcttctgtagcgagtttgtaa

SEQ ID NO: 2 - DNA, 1406 bp, synthetic construct
  misc_feature 1..1406: note = synthetic polymer
  source 1..1406: mol_type = other DNA
aataagaggaagtgaaatctgaataattttgtgttactcatagcgcgtaatatttgtctagggccgcggggactttgaccgtttacgtggagactcgcccaggtgtttttctcaggtgttttccgcgtacgtcggcggctcgtggctctttcgcggcaaaaaggatttggcgcgtaaaagtggttcgagtacgtcggcggctcgtggctctttcgcggcaaaaaggatttggcgcgtaaaagtggttcgaagtacgtcgaccacaaaccccgcccagcgtcttgtcattggcgtcgacgctgtacggggtcaaagttggcgttttattattatagtcagctgacgtgtagtgtatttatacccggtgagttcctcaagaggccactcttgagtgccagcgagtagagttttctcctccgagccgctccgacaccgggactgaaaatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataa

SEQ ID NO: 3 - DNA, 37607 bp, synthetic construct
  misc_feature 1..37607: note = synthetic polymer
  source 1..37607: mol_type = other DNA
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgtacgtcggcggctcgtggctctttcgcggcaaaaaggatttggcgcgtaaaagtggttcgagtacgtcggcggctcgtggctctttcgcggcaaaaaggatttggcgcgtaaaagtggttcgaagtacgtcgaccacaaaccccgcccagcgtcttgtcattggcgtcgacgctgtacggggtcaaagttggcgttttattattatagtcagctgacgtgtagtgtatttatacccggtgagttcctcaagaggccactcttgagtgccagcgagtagagttttctcctccgagccgctccgacaccgggactgaaaatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataaggtgtaaacctgtgattgcgtgtgtggttaacgcctttgtttgctgaatgagttgatgtaagtttaataaagggtgagataatgtttaacttgcatggcgtgttaaatggggcggggcttaaagggtatataatgcgccgtgggctaatcttggttacatctgacctcatggaggcttgggagtgtttggaagatttttctgctgtgcgtaacttgctggaacagagctctaacagtacctcttggttttggaggtttctgtggggctcatcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgtacaggtggctgaactgtatccagaactgagacgcattttgacaattacagaggatgggcaggggctaaagggggtaaagagggagcggggggcttgtgaggctacagaggaggctaggaatctagcttttagcttaatgaccagacaccgtcctgagtgtattacttttcaacagatcaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagatcagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtaaggtttactggccccaattttagcggtacggttttcctggccaataccaaccttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctctttgaaaggtgtaccttgggtatcctgtctgagggtaactccagggtgcgccacaatgtggcctccgactgtggttgcttcatgctagtgaaaagcgtggctgtgattaagcataacatggtatgtggcaactgcgaggacagggcctctcagatgctgacctgctcggacggcaactgtcacctgctgaagaccattcacgtagccagccactctcgcaaggcctggccagtgtttgagcataacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaatcggggttggggttattctctgtcttgtgattctctttattcttatactaacgcttctctgcctaaggctcgccgcctgctgtgtgcacatttgcatttattgtcagctttttaaacgctggggtcgccacccaagatgattaggtacataatcctaggtttactcacccttgcgtcagcccacggtaccacccaaaaggtggattttaaggagccagcctgtaatgttacattcgcagctgaagctaatgagtgcaccactcttataaaatgcaccacagaacatgaaaagctgcttattcgccacaaaaacaaaattggcaagtatgctgtttatgctatttggcagccaggtgacactacagagtataatgttacagttttccagggtaaaagtcataaaacttttatgtatacttttccattttatgaaatgtgcgacattaccatgtacatgagcaaacagtataagttgtggcccccacaaaattgtgtggaaaacactggcactttctgctgcactgctatgctaattacagtgctcgctttggtctgtaccctactctatattaaatacaaaagcagacgcagctttattgaggaaaagaaaatgccttaatttactaagttacaaagctaatgtcaccactaactgctttactcgctgcttgcaaaacaaattcaaaaagttagcattataattagaataggatttaaaccccccggtcatttcctgctcaataccattcccctgaacaattgactctatgtgggatatgctccagcgctacaaccttgaagtcaggcttcctggatgtcagcatctgactttggccagcacctgtcccgcggatttgttccagtccaactacagcgacccaccctaacagagatgaccaacacaaccaacgcggccgccgctaccggacttacatctaccacaaatacaccccaagtttctgcctttgtcaataactgggataacttgggcatgtggtggttctccatagcgcttatgtttgtatgccttattattatgtggctcatctgctgcctaaagcgcaaacgcgcccgaccacccatctatagtcccatcattgtgctacacccaaacaatgatggaatccatagattggacggactgaaacacatgttcttttctcttacagtatgattaaatgagacatgattcctcgagtttttatattactgacccttgttgcgcttttttgtgcgtgctccacattggctgcggtttctcacatcgaagtagactgcattccagccttcacagtctatttgctttacggatttgtcaccctcacgctcatctgcagcctcatcactgtggtcatcgcctttatccagtgcattgactgggtctgtgtgcgctttgcatatctcagacaccatccccagtacagggacaggactatagctgagcttcttagaattctttaattatgaaatttactgtgacttttctgctgattatttgcaccctatctgcgttttgttccccgacctccaagcctcaaagacatatatcatgcagattcactcgtatatggaatattccaagttgctacaatgaaaaaagcgatctttccgaagcctggttatatgcaatcatctctgttatggtgttctgcagtaccatcttagccctagctatatatccctaccttgacattggctggaacgcaatagatgccatgaaccacccaactttccccgcgcccgctatgcttccactgcaacaagttgttgccggcggctttgtcccagccaatcagcctcgcccaccttctcccacccccactgaaatcagctactttaatctaacaggaggagatgactgacaccctagatctagaaatggacggaattattacagagcagcgcctgctagaaagacgcagggcagcggccgagcaacagcgcatgaatcaagagctccaagacatggttaacttgcaccagtgcaaaaggggtatcttttgtctcgtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctccgaggagacaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgtactaagcggtgatgtttctgatcagccaccatgggagtgctaaaattcaagcacatctttttcagaagctttgttaaatcaagtggagtatcccagatagttttcaccttccttctgattccatgttgcttgactctgaatttcagagcacctcctgttattccaaatgtgcctttcctctgggcctggaatgccccaagtgaattttgtcttggaaaatttgatgagccactagatatgagcctcttctctttcataggaagcccccgaataaacgccaccgggcaaggtgttacaatattttatgttgatagacttggctactatccttacatagattcaatcacaggagtaactgtgaatggaggaatcccccagaagatttccttacaagaccatctggacaaagctaagaaagacattacattttatatgccagtagacaatttgggaatggctgttattgactgggaagaatggagacccacttgggcaagaaactggaaacctaaagatgtttacaagaataggtctattgaattggttcagcaacaaaatgtacaacttagtctcacagaggccactgagaaagcaaaacaagaatttgaaaaggcagggaaggatttcctggtagagactataaaattgggaaaattacttcggccaaatcacttgtggggttattatctttttccggattgttacaaccatcactataagaaacccggttacaatggaagttgcttcaatgtagaaataaaaagaaatgatgatctcagctggttgtggaatgaaagcactgctctttacccatccatttatttgaacactcagcagtctcctgtagctgctacactctatgtgcgcaatcgagttcgggaagccatcagagtttccaaaatacctgatgcaaaaagtccacttccggtttttgcatatacccgcatagtttttactgatcaagttttgaaattcctttctcaagatgaacttgtgtatacatttggcgaaactgttgctctgggtgcttctggaattgtaatatggggaaccctcagtataatgcgaagtatgaaatcttgcttgctcctagacaattacatggagactatactgaatccttacataatcaacgtcacactagcagccaaaatgtgtagccaagtgctttgccaggagcaaggagtgtgtataaggaaaaactggaattcaagtgactatcttcacctcaacccagataattttgctattcaacttgagaaaggtggaaagttcacagtacgtggaaaaccgacacttgaagacctggagcaattttctgaaaaattttattgcagctgttatagcaccttgagttgtaaggagaaagctgatgtaaaagacactgatgctgttgatgtgtgtattgctgatggtgtctgtatagatgcttttctaaaacctcccatggagacagaagaacctcaaattttctacaatgcttcaccctccacactatcttaataaactttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatg

SEQ ID NO: 4 - protein, 576 aa, synthetic construct
  REGION 1..576: note = synthetic polymer
MKRARPSEDTFNPVYPYDTETGPPTVPFLTPPFVSPNGFQESPPGVLSLRLSEPLVTSNGMLALKMGNGLSLDEAGNLTSQNVTTVSPPLRGDKSNINLEISAPLTVTSEALTVAAAAPLMVAGNTLTMGSQAPLTVHDSKLSIATQGPLTVSEGKLALQTSGPLTTTDSSTLTITASPPLTTATGSLGIDLKEPIYTQNGKLGLKYGAPLHVTDDLNTLTVATGPGVTINNTSLGTKVTGALGFDSQGNMQLNVAGGLRIDSQNRRLILDVSYPFDAQNQLNLRLGQGPLFINSAHNLDINYNKGLYLFTASNNSKKLEVNLSTAKGLMFDATAIAINAGDGLEFGSPNAPNTNPLKTKIIGHGLEFDSNKAMVPKLGTGLSFDSTGAITVGNKNNDKLTLWTTPAPSPNCDLNAEKDAKLTLVLTKCGSQILATVSVLAVKGSLAPISGTVQSAHLIIRFDENGVLLNNSFLDPEYWNFRNGDLTEGTAYTNAVGFMPNLSAYPKSHGKTAKSNIVSQVYLNGDKTKPVTLTITLNGTQETGDTTPSAYSMSFSWDWSGHNYINEIFATSSYTF

SEQ ID NO: 5 - protein, 9 aa, synthetic construct
  REGION 1..9: note = synthetic polymer
CDCRGDCFC

SEQ ID NO: 6 - protein, 4 aa, synthetic construct
  REGION 1..4: note = synthetic polymer
KKTK

SEQ ID NO: 7 - protein, 4 aa, synthetic construct
  REGION 1..4: note = synthetic polymer
RGDK

SEQ ID NO: 8 - protein, 509 aa, synthetic construct
  REGION 1..509: note = synthetic polymer
MGVLKFKHIFFRSFVKSSGVSQIVFTFLLIPCCLTLNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLSATMFIVSILFLIISSVASL

SEQ ID NO: 9 - DNA, 1473 bp, synthetic construct
  misc_feature 1..1473: note = synthetic polymer
  source 1..1473: mol_type = other DNA
atgggagtgctaaaattcaagcacatctttttcagaagctttgttaaatcaagtggagtatcccagatagttttcaccttccttctgattccatgttgcttgactctgaatttcagagcacctcctgttattccaaatgtgcctttcctctgggcctggaatgccccaagtgaattttgtcttggaaaatttgatgagccactagatatgagcctcttctctttcataggaagcccccgaataaacgccaccgggcaaggtgttacaatattttatgttgatagacttggctactatccttacatagattcaatcacaggagtaactgtgaatggaggaatcccccagaagatttccttacaagaccatctggacaaagctaagaaagacattacattttatatgccagtagacaatttgggaatggctgttattgactgggaagaatggagacccacttgggcaagaaactggaaacctaaagatgtttacaagaataggtctattgaattggttcagcaacaaaatgtacaacttagtctcacagaggccactgagaaagcaaaacaagaatttgaaaaggcagggaaggatttcctggtagagactataaaattgggaaaattacttcggccaaatcacttgtggggttattatctttttccggattgttacaaccatcactataagaaacccggttacaatggaagttgcttcaatgtagaaataaaaagaaatgatgatctcagctggttgtggaatgaaagcactgctctttacccatccatttatttgaacactcagcagtctcctgtagctgctacactctatgtgcgcaatcgagttcgggaagccatcagagtttccaaaatacctgatgcaaaaagtccacttccggtttttgcatatacccgcatagtttttactgatcaagttttgaaattcctttctcaagatgaacttgtgtatacatttggcgaaactgttgctctgggtgcttctggaattgtaatatggggaaccctcagtataatgcgaagtatgaaatcttgcttgctcctagacaattacatggagactatactgaatccttacataatcaacgtcacactagcagccaaaatgtgtagccaagtgctttgccaggagcaaggagtgtgtataaggaaaaactggaattcaagtgactatcttcacctcaacccagataattttgctattcaacttgagaaaggtggaaagttcacagtacgtggaaaaccgacacttgaagacctggagcaattttctgaaaaattttattgcagctgttatagcaccttgagttgtaaggagaaagctgatgtaaaagacactgatgctgttgatgtgtgtattgctgatggtgtctgtatagatgcttttctaaaacctcccatggagacagaagaacctcaaattttctacaatgcttcaccctccacactatcttaa

SEQ ID NO: 10 - protein, 490 aa, synthetic construct
  REGION 1..490: note = synthetic polymer
MGVLKFKHIFFRSFVKSSGVSQIVFTFLLIPCCLTLNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 11 - DNA, 20 bp, synthetic construct
  misc_feature 1..20: note = synthetic polymer
  source 1..20: mol_type = other DNA
atcgaagaggtactggctga

SEQ ID NO: 12 - DNA, 20 bp, synthetic construct
  misc_feature 1..20: note = synthetic polymer
  source 1..20: mol_type = other DNA
cctccggtgataatgacaag

SEQ ID NO: 13 - DNA, 20 bp, synthetic construct
  misc_feature 1..20: note = synthetic polymer
  source 1..20: mol_type = other DNA
gagggtaactccagggtgcg

SEQ ID NO: 14 - DNA, 24 bp, synthetic construct
  misc_feature 1..24: note = synthetic polymer
  source 1..24: mol_type = other DNA
tttcactagcatgaagcaaccaca

SEQ ID NO: 15 - DNA, 22 bp, synthetic construct
  misc_feature 1..22: note = synthetic polymer
  source 1..22: mol_type = other DNA
ggacaggagaggaagaccatgt

SEQ ID NO: 16 - DNA, 24 bp, synthetic construct
  misc_feature 1..24: note = synthetic polymer
  source 1..24: mol_type = other DNA
tggagtggtagaattctgtcatgc

SEQ ID NO: 17 - DNA, 20 bp, synthetic construct
  misc_feature 1..20: note = synthetic polymer
  source 1..20: mol_type = other DNA
ccaggcaggcttttcattca

SEQ ID NO: 18 - DNA, 20 bp, synthetic construct
  misc_feature 1..20: note = synthetic polymer
  source 1..20: mol_type = other DNA
aggtcctggaagtatgggtg

SEQ ID NO: 19 - DNA, 20 bp, synthetic construct
  misc_feature 1..20: note = synthetic polymer
  source 1..20: mol_type = other DNA
tacacactccttgctcctgg

SEQ ID NO: 20 - DNA, 20 bp, synthetic construct
  misc_feature 1..20: note = synthetic polymer
  source 1..20: mol_type = other DNA
cttagtctcacagaggccac

SEQ ID NO: 21 - DNA, 23 bp, synthetic construct
  misc_feature 1..23: note = synthetic polymer
  source 1..23: mol_type = other DNA
aggtggctctgaagatcaatctg

SEQ ID NO: 22 - DNA, 20 bp, synthetic construct
  misc_feature 1..20: note = synthetic polymer
  source 1..20: mol_type = other DNA
ttagccctccggttcttgaa

(19) United States
(12) Patent Application Publication
(10) Pub. No.: US 2026/0335388 A1
(43) Pub. Date: Oct. 29, 2026

(54) COMBINATION THERAPIES WITH ONCOLYTIC ADENOVIRUS AND TOPOISOMERASE I INHIBITORS OR PRODRUGS THEREOF

(71) Applicants: Theriva Biologics, S.L., Barcelona (ES); Hospital Sant Joan de Déu, Esplugues de Llobregat (ES)

(72) Inventors: Ángel Montero CARCABOSO, Barcelona (ES); Víctor Burgueño SANDOVAL, Barcelona (ES); Manel Maria CASCALLO PIQUERAS, Gelida (ES); Ana MATO BERCIANO, Rockville, MD (US); Sheila CONNELLY, Rockville, MD (US)

(21) Appl. No.: 19/112,372

(22) PCT Filed: Sep. 18, 2023
(86) PCT No.: PCT/IB2023/059239

§ 371 Date: Mar. 17, 2025

Related U.S. Application Data
(60) Provisional application No. 63/408,079, filed on Sep. 19, 2022.
(60) Provisional application No. 63/533,176, filed on Aug. 17, 2023.

(51) Int. Cl. A61K 35/768; A61K 31/4745; A61K 45/06; A61P 35/00; C12N 7/00; C12N 9/26

(52) U.S. Cl. CPC A61K 35/768; A61K 31/4745; A61K 45/06; A61P 35/00; C12N 7/00; C12N 9/2474; C12N 2710/10032; C12Y 302/01035

(57) ABSTRACT

The present disclosure relates, inter alia, to combination therapies of an oncolytic adenovirus with a topoisomerase I inhibitor or prodrug thereof, such as topotecan or SN-38, or a prodrug of a topoisomerase inhibitor, such as irinotecan, for treating retinoblastoma, Ewing sarcoma, or neuroblastoma.

### CROSS-REFERENCE TO RELATED APPLICATIONS

This application claims the benefit of U.S. Provisional Application No. 63/408,079, filed Sep. 19, 2022, and U.S. Provisional Application No. 63/533,176, filed Aug. 17, 2023, the entire contents of which are hereby incorporated by reference in their entireties.

### TECHNICAL FIELD

The present disclosure relates, inter alia, to combination therapies of an oncolytic adenovirus with topotecan, irinotecan, or SN-38 for use in oncology.

### DESCRIPTION OF THE XML FILE SUBMITTED ELECTRONICALLY

The instant application contains a Sequence Listing which has been submitted electronically in XML file format and is hereby incorporated by reference in its entirety. Said XML file, created on Sep. 8, 2023, is named “VCN-004_Sequence_Listing.xml” and is 72,036 bytes in size.

### BACKGROUND

Retinoblastoma, neuroblastoma, and Ewing sarcoma are common types of solid tumors, especially in children. Retinoblastoma is the most common malignant intraocular tumor in children, with an incidence of 1/17,000 newborns. It develops in the retina, usually growing towards the vitreous cavity.

Current treatment of retinoblastoma is aimed primarily at the patient's survival and secondly at preservation of sight. There are various therapeutic options, including: systemic or local ocular chemotherapy, radiotherapy, cryotherapy, laser therapy and surgery (enucleation of the affected eye). The choice of therapeutic regimen depends on many factors, such as the stage of tumor development, whether the tumor is unifocal or multifocal and unilateral or bilateral, and the site and size of the tumor.

In the late 1990s, there was a shift from local, eye-directed therapies such as external beam radiotherapy (EBRT), which has been consistently associated with a high risk of second malignancies, toward systemic chemotherapy combined with aggressive focal therapies (Rodriguez-Galindo et al., “Treatment of retinoblastoma: current status and future perspectives,” Curr Treat Options Neurol. 2007 July; 9(4):294-307). The use of systemic chemoreduction was effective for tumor control in eyes with less advanced tumors, avoiding the use of EBRT, but approximately 75% of retinoblastoma cases worldwide present with more advanced tumors with massive vitreous or subretinal seeding and retinal detachment, where both radiation and systemic chemotherapy can rarely save the eye or vision. Systemic chemoreduction results in reduced long-term toxicity by avoiding EBRT in many patients, but its acute toxicity can be fatal in up to 4-5% of patients in non-developed countries. Furthermore, studies show an additive risk for secondary malignancies when chemotherapy is combined with EBRT. Ototoxicity caused by carboplatin and cases of fatal chemotherapy-induced leukemia have also been reported in non-irradiated children with retinoblastoma. Thus, in the mid-2000s, physicians moved away from systemic chemotherapy to advanced techniques for more selective ocular delivery to increase drug exposure in the tumor, maximizing efficacy and minimizing the probability of adverse events. The introduction of ophthalmic artery chemotherapy (OAC) led to great success in treating eyes with more advanced disease in which systemic chemotherapy had poorer results. The current standard therapy in high- and middle-income countries is intravitreous chemotherapy in conjunction with OAC, which has led to improvement in eye preservation and eliminated the use of EBRT.

Among the antineoplastic agents used for retinoblastoma, topotecan has been used based on its effect in preclinical models and clinical studies. Topotecan alone or in combination is active against retinoblastoma (Schaiquevich et al., “Ocular pharmacology of topotecan and its activity in retinoblastoma,” Retina. 2014 September; 34(9):1719-27). It shows a favorable passage to the vitreous when given intravenously and intraarterially, and ocular toxicity is minimal by all routes of administration. However, its clinical role, optimal dose, and route of administration for the treatment of retinoblastoma remain to be determined. As an example, a study of OAC administration of topotecan showed that the OAC route selectively delivers chemotherapy to the eye, and that topotecan concentrations in the vitreous and retina of non-tumor bearing pigs were 243 and 146 times higher after OAC than after IV, respectively, while systemic exposure was comparable between the two routes (Schaiquevich et al., “Treatment of Retinoblastoma: What Is the Latest and What Is the Future,” Front Oncol. 2022 Apr. 1; 12:822330). The favorable disposition of topotecan in ocular tissues after OAC would have predicted successful tumor control using OAC topotecan; however, this has not been clinically observed. This failure to achieve adequate tumor control despite topotecan vitreous levels above the IC50 may be related to the administration schedule. Topotecan exerts its cytotoxic activity by inhibiting the nuclear enzyme topoisomerase I, resulting in double-strand breaks during cell replication, and thus it acts on cells in S-phase. Therefore, subsequent doses are required to target quiescent tumor cells that enter cell replication, and protracted schedules have been associated with greater antitumor effects compared to intermittent, higher-dose schedules. However, repeated daily OAC to deliver topotecan is impractical, limiting its efficacy via this delivery route.

In view of the above, despite the advancements in retinoblastoma treatment, eyes that relapse or that are initially refractory to conventional therapy are still difficult to treat with currently available drug options.

Ewing sarcoma is the second most common bone cancer in children. It occurs most frequently in the long bones of the legs or arms, the pelvis, chest wall, spine and the skull, but can also begin in the soft tissues and not involve bone. This disease most often occurs in adolescents, with nearly half of cases arising between the ages of 10 and 20. Ewing sarcoma is somewhat more common in males than in females.

In children and adolescents, oncologists typically prescribe a combination of five drugs given in an alternating order for up to a year. This combination therapy is able to cure around 70% of young patients with localized disease. But if the cancer is metastatic, does not respond to treatment, or relapses, then prognosis tends to be poor.

Variations of this combination therapy are used for adults, as well, but often adults cannot tolerate a full year of the same intensive treatment given to children. This may partially explain why adult patients with Ewing's sarcoma have lower rates of cure than younger patients.

Neuroblastoma accounts for 3.8% of all childhood cancer diagnoses. There are over 650 cases each year in the U.S. Neuroblastoma most often originates in the adrenal glands, which are located on top of each kidney. However, tumors can begin anywhere in the body. Other common sites are the chest, neck, and pelvis. While it may be found in only one spot in the body at the time of diagnosis in some patients, in others the cancer may have metastasized from its primary location to the lymph nodes, bone marrow, or bones.

Children with neuroblastoma have widely divergent outcomes, ranging from cure in >90% of patients with low risk disease to <50% for those with high risk disease. Recent research has shed light on the biology of neuroblastoma, allowing for more accurate risk stratification and treatment reduction in many cases, although newer treatment strategies for children with high-risk and relapsed neuroblastoma are needed to improve outcomes.

Thus, there remains a need for alternative and more effective therapies to treat retinoblastoma, Ewing sarcoma, and neuroblastoma. In the case of retinoblastoma, such alternative therapies are needed to avoid enucleating one or both eyes. New therapeutic alternatives are also required to allow the risk of the patient developing metastases, secondary malignancies, or recurrent disease to be eliminated or reduced.

### SUMMARY

Accordingly, in aspects, the disclosure provides methods for treating retinoblastoma, Ewing sarcoma, or neuroblastoma in a patient in need thereof, comprising co-administering to the patient (i) an oncolytic adenovirus comprising replication machinery specific for tumor cells and, optionally, a polynucleotide sequence encoding a hyaluronidase enzyme inserted into its genome and (ii) a topoisomerase I inhibitor or a prodrug of a topoisomerase inhibitor.

In embodiments, the oncolytic adenovirus is administered by intraocular, intrathecal, intravitreal, intravenous, intraarterial, or intratumoral injection. In embodiments, the topoisomerase I inhibitor or a prodrug thereof is administered intravenously, intraarterially, intravitreally, intrathecally, intraventricularly, or orally.

In embodiments, the oncolytic adenovirus and the topoisomerase I inhibitor or a prodrug thereof are formulated in separate compositions. In embodiments, the oncolytic adenovirus and the topoisomerase I inhibitor or a prodrug thereof are formulated in a single composition. In embodiments, the separate compositions are administered simultaneously or contemporaneously. In embodiments, the oncolytic adenovirus is administered first and the topoisomerase I inhibitor or a prodrug thereof is administered within about 60 minutes of the administration of the oncolytic adenovirus. In embodiments, the topoisomerase I inhibitor or a prodrug thereof is administered within about 30 minutes, within about 20 minutes, within about 10 minutes, within about 5 minutes, or within about 1 minute of the administration of the oncolytic adenovirus. In embodiments, the topoisomerase I inhibitor or a prodrug thereof is administered first and the oncolytic adenovirus is administered within about 60 minutes of the administration of the oncolytic adenovirus. In embodiments, the oncolytic adenovirus is administered within about 30 minutes, within about 20 minutes, within about 10 minutes, within about 5 minutes, or within about 1 minute of the administration of the topoisomerase I inhibitor or a prodrug thereof.

In embodiments, the subject is administered one intrathecal injection of the oncolytic adenovirus concomitantly or contemporaneously with systemic or intrathecal cycles of the topoisomerase I inhibitor or prodrug thereof. In embodiments, the subject is administered the oncolytic adenovirus and the topoisomerase I inhibitor or prodrug thereof systemically. In embodiments, the oncolytic adenovirus and/or topoisomerase I inhibitor or prodrug thereof is co-administered with one or more local and/or systemic corticosteroids. In embodiments, the one or more local and/or systemic corticosteroids are selected from hydrocortisone, prednisone, prednisolone, methylprednisolone, dexamethasone, betamethasone, alsosterone, budesonide, fluticasone, flunisolide, ciclesonide, mometasone, beclomethasone, triamcinolone, and tixocortol. In embodiments, the one or more local and/or systemic corticosteroids are selected from methylprednisolone, dexamethasone, betamethasone, and triamcinolone.

In embodiments, the topoisomerase I inhibitor is topotecan, or SN-38, or the SN-38 prodrug irinotecan, which is converted to SN-38 in vivo.

In embodiments, the hyaluronidase enzyme is the human hyaluronidase enzyme PH20. In embodiments, the sequence that encodes a hyaluronidase enzyme is SEQ ID NO: 9, or a sequence having at least about 75%, at least about 80%, at least about 85%, at least about 90%, at least about 95%, at least about 98%, or at least about 99% sequence identity thereto.

In embodiments, the oncolytic adenovirus is generated from a human adenovirus serotype 5.

In embodiments, oncolytic adenoviral replication occurs in tumor cells with an aberrant Rb-E2F pathway (e.g. a solid tumor) and not in healthy, non-tumor, or normal cells. In embodiments, the oncolytic adenovirus is engineered to replicate in tumor cells and not healthy, non-tumor, or normal cells by deletion of the Rb binding domain in the sequence coding for the E1a protein and the insertion of four binding sites to E2F-1 and one binding site to Sp1 into the endogenous promoter of E1a to control the expression of E1a.

In embodiments, oncolytic adenoviral replication occurs in tumor cells with an aberrant Rb-E2F pathway (e.g. a solid tumor) and not in healthy, non-tumor, or normal cells. In embodiments, the oncolytic adenovirus is engineered to replicate in tumor cells and not healthy, non-tumor, or normal cells by deletion Δ24 in the sequence coding for the E1a protein and the insertion of four binding sites to E2F-1 and one binding site to Sp1 into the endogenous promoter of E1a to control the expression of E1a.

In embodiments, the oncolytic adenovirus has the capsid modified such that the heparan sulfate binding domain ⁹¹KKTK⁹⁴ (SEQ ID NO: 8) present in the adenovirus fiber has been replaced.

In embodiments, the oncolytic adenovirus has the capsid modified such that the heparan sulfate binding domain ⁹¹KKTK⁹⁴ (SEQ ID NO: 8) present in the adenovirus fiber has been replaced by the domain 91RGDK⁹⁴ (SEQ ID NO: 9).

In embodiments, the oncolytic adenovirus is VCN-01 (SEQ ID NO: 3), or a functional variant thereof.

In embodiments, the patient is a human patient. In embodiments, the human patient is a pediatric human patient.

In embodiments, the retinoblastoma, Ewing sarcoma, or neuroblastoma is a retinoblastoma, Ewing sarcoma, or neuroblastoma resistant to conventional chemotherapy and/or radiotherapy treatment.

In embodiments, the method improves and/or increases and/or enhances antitumor efficacy compared to treatment with the topoisomerase I inhibitor or a prodrug thereof without the oncolytic adenovirus. In embodiments, the method results in reduced or maintained tumor size, and/or prevention or reduction of metastasis, secondary malignancy, or trilateral retinoblastoma associated with retinoblastoma, compared to treatment with the topoisomerase I inhibitor or a prodrug thereof without the oncolytic adenovirus. In embodiments, the method results in reduced or maintained tumor size, and/or prevention or reduction of metastasis, secondary malignancy, or recurrent disease associated with Ewing sarcoma, compared to treatment with the topoisomerase I inhibitor or a prodrug thereof without the oncolytic adenovirus. In embodiments, the method results in reduced or maintained tumor size, and/or prevention or reduction of metastasis, secondary malignancy, or recurrent disease associated with neuroblastoma, compared to treatment with the topoisomerase I inhibitor or a prodrug thereof without the oncolytic adenovirus.

In embodiments, without wishing to be bound by theory, administration of the oncolytic adenovirus reduces apoptotic action of topotecan as compared to monotherapy. In embodiments, without wishing to be bound by theory, administration of topotecan following administration of the oncolytic adenovirus leads to S-phase cell cycle arrest. In embodiments, without wishing to be bound by theory, the S-phase cell cycle arrest results in increased infectivity of the oncolytic adenovirus as compared to treatment with the oncolytic adenovirus without the topoisomerase I inhibitor or prodrug thereof. In embodiments, without wishing to be bound by theory, administration of topotecan following administration of the oncolytic adenovirus leads to increased E2F-1, p21 and/or cyclin E1 expression as compared to treatment with the oncolytic adenovirus without the topoisomerase I inhibitor or prodrug thereof. In embodiments, without wishing to be bound by theory, the increased E2F-1 expression results in increased oncolytic activity of the oncolytic adenovirus as compared to treatment with the oncolytic adenovirus without the topoisomerase I inhibitor or prodrug thereof. In embodiments, without wishing to be bound by theory, the increased infectivity and oncolytic activity of the oncolytic adenovirus occurs without substantially increasing replication of the oncolytic adenovirus. In embodiments, without wishing to be bound by theory, sequential treatments in which the oncolytic adenovirus is administered first, followed by systemic topotecan, enhance or increase cell infection and/or anticancer efficacy of the oncolytic adenovirus.

In aspects, the disclosure provides methods for treating retinoblastoma, Ewing sarcoma, or neuroblastoma in a patient in need thereof, comprising administering to the patient (i) an oncolytic adenovirus comprising replication machinery specific for tumor cells and, optionally, a polynucleotide sequence encoding a hyaluronidase enzyme inserted into its genome, and (ii) a topoisomerase I inhibitor or a prodrug thereof.

In embodiments, the oncolytic adenovirus is administered by intraocular, intrathecal, intravitreal, intravenous, intraarterial, or intratumoral injection. In embodiments, the topoisomerase I inhibitor or a prodrug thereof is administered intravenously, intravitreally, intrathecally, intraventricularly, or orally.

In embodiments, the oncolytic adenovirus and the topoisomerase I inhibitor or a prodrug thereof are formulated in separate compositions. In embodiments, the oncolytic adenovirus and the topoisomerase I inhibitor or a prodrug thereof are formulated in a single composition. In embodiments, the oncolytic adenovirus is administered first and the topoisomerase I inhibitor or a prodrug thereof is administered within about 12 weeks of the administration of the oncolytic adenovirus. In embodiments, the topoisomerase I inhibitor or a prodrug thereof is administered within about 60 minutes, about 2 hours, about 3 hours, about 4 hours, about 5 hours, about 6 hours, about 7 hours, about 8 hours, about 9 hours, about 10 hours, about 11 hours, about 12 hours, about 1 day, about 2 days, about 3 days, about 4 days, about 5 days, about 6 days, about 7 days, about 8 days, about 9 days, about 10 days, about 11 days, about 12 days, about 13 days, about 2 weeks, about 3 weeks, about 4 weeks, about 5 weeks, about 6 weeks, about 7 weeks, about 8 weeks, about 9 weeks, about 10 weeks, or about 11 weeks of the administration of the oncolytic adenovirus.

In embodiments, the subject is administered one intrathecal injection of the oncolytic adenovirus concomitantly or contemporaneously with systemic or intrathecal cycles of the topoisomerase I inhibitor or prodrug thereof. In embodiments, the subject is administered the oncolytic adenovirus and the topoisomerase I inhibitor or prodrug thereof systemically. In embodiments, the oncolytic adenovirus and/or topoisomerase I inhibitor or prodrug thereof is co-administered with one or more local and/or systemic corticosteroids. In embodiments, the one or more local and/or systemic corticosteroids are selected from hydrocortisone, prednisone, prednisolone, methylprednisolone, dexamethasone, betamethasone, alsosterone, budesonide, fluticasone, flunisolide, ciclesonide, mometasone, beclomethasone, triamcinolone, and tixocortol. In embodiments, the one or more local and/or systemic corticosteroids are selected from methylprednisolone, dexamethasone, betamethasone, and triamcinolone.

In embodiments, the topoisomerase I inhibitor or prodrug thereof is topotecan, SN-38, or irinotecan.

In embodiments, the hyaluronidase enzyme is the human hyaluronidase enzyme PH20. In embodiments, the sequence that encodes a hyaluronidase enzyme is SEQ ID NO: 9, or a sequence having at least about 75%, at least about 80%, at least about 85%, at least about 90%, at least about 95%, at least about 98%, or at least about 99% sequence identity thereto.

In embodiments, the oncolytic adenovirus is generated from a human adenovirus serotype 5.

In embodiments, oncolytic adenoviral replication occurs in tumor cells with an aberrant Rb-E2F pathway and not in healthy, non-tumor, or normal cells. In embodiments, the oncolytic adenovirus is engineered to replicate in tumor cells and not healthy, non-tumor, or normal cells by deletion of the Rb binding domain in the sequence coding for the E1a protein and the insertion of four binding sites to E2F-1 and one binding site to Sp1 into the endogenous promoter of E1a to control the expression of E1a.

In embodiments, oncolytic adenoviral replication occurs in tumor cells with an aberrant Rb-E2F pathway and not in healthy, non-tumor, or normal cells. In embodiments, the oncolytic adenovirus is engineered to replicate in tumor cells and not healthy, non-tumor, or normal cells by deletion Δ24 in the sequence coding for the E1a protein and the insertion of four binding sites to E2F-1 and one binding site to Sp1 into the endogenous promoter of E1a to control the expression of E1a.

In embodiments, the oncolytic adenovirus has the capsid modified such that the heparan sulfate binding domain ⁹¹KKTK⁹⁴ (SEQ ID NO: 8) present in the adenovirus fiber has been replaced. In embodiments, the oncolytic adenovirus has the capsid modified such that the heparan sulfate binding domain ⁹¹KKTK⁹⁴ (SEQ ID NO: 8) present in the adenovirus fiber has been replaced by the domain ⁹¹RGDK⁹⁴ (SEQ ID NO: 9).

In embodiments, the oncolytic adenovirus is VCN-01 (SEQ ID NO: 3), or a functional variant thereof.

In embodiments, the patient is a human patient. In embodiments, the human patient is a pediatric human patient.

In embodiments, the retinoblastoma, Ewing sarcoma, or neuroblastoma is a retinoblastoma, Ewing sarcoma, or neuroblastoma resistant to conventional chemotherapy and/or radiotherapy treatment.

In embodiments, the method improves and/or increases and/or enhances antitumor efficacy compared to treatment with the topoisomerase I inhibitor or a prodrug thereof without the oncolytic adenovirus. In embodiments, the method results in reduced or maintained tumor size, and/or prevention or reduction of metastasis, secondary malignancy, or trilateral retinoblastoma associated with retinoblastoma, compared to treatment with the topoisomerase I inhibitor or a prodrug thereof without the oncolytic adenovirus. In embodiments, the method results in reduced or maintained tumor size, and/or prevention or reduction of metastasis, secondary malignancy, or recurrent disease associated with Ewing sarcoma, compared to treatment with the topoisomerase I inhibitor or a prodrug thereof without the oncolytic adenovirus. In embodiments, the method results in reduced or maintained tumor size, and/or prevention or reduction of metastasis, secondary malignancy, or recurrent disease associated with neuroblastoma, compared to treatment with the topoisomerase I inhibitor or a prodrug thereof without the oncolytic adenovirus.

In embodiments, without wishing to be bound by theory, administration of the oncolytic adenovirus reduces apoptotic action of topotecan as compared to monotherapy. In embodiments, without wishing to be bound by theory, administration of topotecan following administration of the oncolytic adenovirus leads to S-phase cell cycle arrest. In embodiments, without wishing to be bound by theory, the S-phase cell cycle arrest results in increased infectivity of the oncolytic adenovirus as compared to treatment with the oncolytic adenovirus without the topoisomerase I inhibitor or prodrug thereof. In embodiments, without wishing to be bound by theory, administration of topotecan following administration of the oncolytic adenovirus leads to increased E2F-1, p21 and/or cyclin E1 expression as compared to treatment with the oncolytic adenovirus without the topoisomerase I inhibitor or prodrug thereof. In embodiments, without wishing to be bound by theory, the increased E2F-1 expression results in increased oncolytic activity of the oncolytic adenovirus as compared to treatment with the oncolytic adenovirus without the topoisomerase I inhibitor or prodrug thereof. In embodiments, without wishing to be bound by theory, the increased infectivity and oncolytic activity of the oncolytic adenovirus occurs without substantially increasing replication of the oncolytic adenovirus. In embodiments, without wishing to be bound by theory, sequential treatments in which the oncolytic adenovirus is administered first, followed by systemic topotecan, enhance or increase cell infection and/or anticancer efficacy of the oncolytic adenovirus.

### BRIEF DESCRIPTION OF THE DRAWINGS

FIG. 1 shows an immunoblot of E1a in retinoblastoma cells infected with VCN-01 (SEQ ID NO: 3) and treated with topotecan, carboplatin, or melphalan.

FIGS. 2A-B show the intratumor levels of VCN-01 at day 5 (FIG. 2A) and at day 15 (FIG. 2B) quantified after direct injection of VCN-01 and systemic treatment with topotecan, carboplatin, and hydroxyurea in Y79 tumors implanted in the subcutaneous of nude mice.

FIG. 3 shows the intratumor levels of VCN-01 at day 5 quantified after direct injection of VCN-01 and systemic treatment with topotecan, carboplatin, and hydroxyurea in HSJD-RBT-7 tumors implanted in the subcutaneous of nude mice.

FIGS. 4A-C shows an E1a immunoblot (FIG. 4A) or an histological E1a immunostaining (FIG. 4B) from Y79 tumors implanted in the subcutaneous of nude mice after direct injection with VCN-01 and systemic treatment with topotecan, carboplatin, and hydroxyurea. FIG. 4C shows the quantification of E1A-positive cell counts in the immunoblotting of FIG. 4B.

FIG. 5 shows the antitumor activity (depicted as survival percentage in a Kaplan-Meier curve) observed after treatment with topotecan+VCN-01 in Y79 tumors implanted in the subcutaneous of nude mice, compared to treatments with either VCN-01 alone or topotecan alone.

FIGS. 6A-B shows the antitumor activity (depicted as survival percentage in a Kaplan-Meier curve) observed after treatment with topotecan+VCN-01 in Y79 tumors implanted orthotopically in mice eye (FIG. 6A) or HSJD-RBT-7 orthotopic tumors (FIG. 6B), compared to treatments alone.

FIG. 7 shows an E1a immunoblot of Y79 tumors grown in the subcutaneous of nude mice treated with VCN-01 alone or in combination with carboplatin, melphalan, hydroxyurea, etoposide, irinotecan, and SN-38.

FIG. 8 shows the antitumor activity (depicted as survival percentage in a Kaplan-Meier curve) observed after treatment with intrathecal VCN-01+systemic topotecan in HSJD-RBT-7 tumors implanted orthotopically in the brain (intraventricular) of mice, compared to treatments with VCN-01 alone, topotecan alone, and standard of care treatment.

FIG. 9 shows the body weight of animals after treatment with intrathecal VCN-01+systemic topotecan in HSJD-RBT-7 tumors implanted orthotopically in the brain (intraventricular) of mice compared to VCN-01 treatment alone.

FIG. 10 shows the tumor load observed after treatment with intrathecal VCN-01+systemic topotecan in HSJD-RBT-7 tumors implanted orthotopically in the brain (intraventricular) of mice, compared to treatments with VCN-01 alone, topotecan alone, and standard of care treatment.

FIG. 11 shows the growth curve of HSJD-ES-033 Ewing sarcoma tumors implanted subcutaneously in nude mice after treatment with intratumoral injection of VCN-01+systemic topotecan combination, compared to VCN-01 treatment alone.

FIG. 12 shows the growth curve of patient-derived neuroblastoma tumors implanted subcutaneously in nude mice after treatment with intratumoral injection of VCN-01+systemic topotecan or irinotecan combinations), compared to treatments with VCN-01 alone, topotecan alone, or irinotecan alone.

FIGS. 13A-H show the interaction of VCN-01 and chemotherapeutics in vitro. FIG. 13A shows antiproliferative activity of topotecan (TPT), carboplatin (CBP), and melphalan (MEL), with VCN-01 at 10 MOI (VCN-01), or without VCN-01 (Medium) in Y79 cells. Values are means and SD of three replicates. Lines are the best fitting curves built using the least square regression method of GraphPad. Cell viability is expressed as the relative percentage of the assay signal of treated cells, compared to control untreated cells, which was set as 100%. FIG. 13B shows immunoblotting of p53, cPARP and E1A following treatment of Y79 cells with VCN-01 (48 h; 50 MOI), TPT (24 h; 2 μM), VCN-01 and TPT combination (VCN-01/TPT; 48 h; TPT added at 24 h), CBP (24 h; 12.5 μM), VCN-01 and CBP combination (VCN-01/CBP; 48 h; CBP added at 24 h), MEL (24 h; 12 μM), and VCN-01 and MEL combination (VCN-01/MEL; 48 h; MEL added at 24 h). Control cells were not treated. GAPDH was the loading control. FIG. 13C shows representative bright-field images of Y79 cells (floating aggregates) treated with VCN-01 (48 h; 50 MOI), TPT (48 h; 2 μM) or the combination (48 h; TPT added at 24 h). FIG. 13D shows viability of Y79 cells treated for 6 days with the sequences “TPT first” or “VCN-01 first”. Treatments (TPT, 2 M; VCN-01, 50 MOI; or Medium) and sequences (in which the second treatment is administered at the beginning of day 4) are detailed below the columns. Bars are mean and SD of 8 replicates. The MTS signal of control untreated cells was set as 100%. FIG. 13E shows immunoblotting of p53, cPARP, E2F-1 and E1A following treatment of Y79 cells with VCN-01 (24 h or 48 h; 50 MOI), TPT (24 h or 48 h; 2 μM) or the combinations at the sequences “TPT first” (TPT/VCN-01; 48 h; VCN-01 added at 24 h) or “VCN-01 first” (VCN-01/TPT; 48 h; TPT added at 24 h). β-tubulin was the loading control. FIG. 13F shows quantification of E1A mRNA in Y79 cells treated with the sequences “TPT first” or “VCN-01 first”. Dots are experimental replicates and bars are mean and SD. FIG. 13G shows quantification of E1B mRNA in Y79 cells treated with the sequences “TPT first” or “VCN-01 first”. FIG. 13H shows immunoblotting of E2F-1 and E1A following treatment of Y79 cells exposed to VCN-01 for 48 h (50 MOI). At 24 h, TPT (2 μM), CBP (12.5 μM), MEL (12 μM), hydroxyurea (HU; 100 μM), etoposide (ETO; 2 μM), irinotecan (IRN; 10 μM) or SN-38 (1 μM) was added. GAPDH was the loading control.

FIG. 14 shows antiproliferative activity of topotecan, carboplatin, melphalan, combined with 10 MOI of VCN-01, or as single agents (Medium) in RBT-5, RBT-7 and RBVS-10 cells. Values are means and SD of three replicates. Lines are the best fitting curves built using GraphPad. Cell viability is expressed as the relative percentage of the assay signal of treated cells, compared to control untreated cells, which were set as 100%.

FIGS. 15A-C show interaction of VCN-01 and chemotherapeutics in vitro. FIG. 15A shows immunoblotting of p53, cPARP and E1A following treatment of primary retinoblastoma cells (RBT-5, RBT-7 and RBVS-10) with VCN-01 (48 h; 50 MOI), topotecan (TPT; 24 h; 2 M), VCN-01 and TPT combination (VCN-01/TPT; 48 h; TPT added at 24 h), carboplatin (CBP; 24 h; 12.5 M), VCN-01 and CBP combination (VCN-01/CBP; 48 h; CBP added at 24 h), melphalan (MEL; 24 h; 12 μM), and VCN-01 and MEL combination (VCN-01/MEL; 48 h; MEL added at 24 h). Control cells were not treated. GAPDH was the loading control. FIG. 15B shows proportion (%) of cPARP⁺Y79 and RBT-7 cells treated with VCN-01 (48 h; 50 MOI), topotecan (24 h; 2 μM) or the combination (VCN-01/TPT; 48 h; TPT added at 24 h). Dots are experimental replicates and bars are mean and SD. FIG. 15C shows representative flow cytometry histograms of cPARP expression in Y79 and RBT-7 cells.

FIG. 16 shows immunoblotting of topoisomerase I (TOPO 1) following treatment of primary retinoblastoma cells (RBT-7 and RBVS-10) with VCN-01 (48 h; 50 MOI), topotecan (TPT; 24 h; 2 μM), VCN-01 and TPT combination (VCN-01/TPT; 48 h; TPT added at 24 h), carboplatin (CBP; 24 h; 12.5 μM), VCN-01 and CBP combination (VCN-01/CBP; 48 h; CBP added at 24 h), melphalan (MEL; 24 h; 12 μM), and VCN-01 and MEL combination (VCN-01/MEL; 48 h; MEL added at 24 h). Control cells were not treated. GAPDH was the loading control.

FIGS. 17A-G show cell cycle and E2F-1 expression in retinoblastoma cells exposed to chemotherapeutics. FIG. 17A shows percentage of Y79 cells in G2, S and G1 cell cycle phases following 24 h treatment with culture medium (Control), topotecan (TPT; 2 μM), carboplatin (CBP; 12.5 μM) or hydroxyurea (HU; 100 μM). Bars are means and SD of three replicates. FIG. 17B shows representative images of the cell cycle evaluation of Y79 cells treated with TPT (2 μM) for 4, 16 or 24 h. In the plots, G1-phase cells are colored in green (left peak). S-phase cells are in yellow (middle) and G2-phase cells are in blue (right). FIG. 17C shows percentage of Y79 cells in G2, S and G1 cell cycle phases following treatment with TPT (2 μM) for up to 48 h. Bars are means and SD of three replicates. FIG. 17D shows immunoblotting of E2F-1, Cyclin E1 and p21 following up to 48 h treatment of Y79 cells with TPT (2 μM), CBP (12.5 μM), MEL (12 μM), HU (100 μM) or SN-38 (1 μM). GAPDH was the loading control. FIG. 17E shows immunostaining of E2F-1 (nuclear staining in brown) in Y79 intraocular xenografts treated with TPT (0.6 mg/kg, daily for five consecutively days), or with saline. FIG. 17F shows expression of cyclins and cell cycle regulation genes induced by TPT (2 μM; 24 h) and CBP (12.5 UM; 24 h) in RBT-7 cells infected with VCN-01 (50 MOI; 48 h). Values are fold-changes relative to expression in control infected cells exposed to culture medium. FIG. 17G shows expression of CDKN1A and CDK6 following 48 h treatment of VCN-01-infected RBT-7 cells with TPT (2 μM) or CBP (12.5 μM). Values are fold-changes relative to expression in control infected cells exposed to culture medium.

FIGS. 18A-J show effect of chemotherapeutics on adenoviral transduction and replication in retinoblastoma. FIG. 18A shows representative plots of the percentage of Y79 cells in G2, S and G1 cell cycle phases following treatment with hydroxyurea (HU; 4 mM) (Synchronized) or culture medium (Asynchronized). The table contains the quantification of the plots. FIG. 18B shows representative bright light and fluorescence images of Y79 cells pre-exposed to 4 mM HU (Synchronized) and treated with AdTLRGDK (50 MOI) for 24, 48 or 72 h. Culture medium was used as control (Asynchronized). FIG. 18C shows representative pseudo-color-flow-plots of the experiment of panel B. Non infected cells were used as negative control. FIG. 18D shows counts (%) of GFP⁺ cells of the experiment of panel B. Dots are experimental replicates (n=3-4) and bars are mean and SD. FIG. 18E shows proportion (%) of GFP⁺Y79 cells pretreated for 24 h with AdTLRGDK (50 MOI) and exposed for additional 24 h to culture medium (Medium), topotecan (TPT; 2 μM), carboplatin (CBP; 12.5 μM), melphalan (MEL; 12 μM) or HU (100 μM), or exposed to the reverse sequence in which cells were treated with TPT or HU 6 h before AdTLRGDK infection. Dots are experimental replicates and bars are mean and SD. FIG. 18F shows effect of TPT (2 μM) on the viral production of VCN-01 in Y79 cells, measured as the number of viral genomes per mL of cell pellet extract. FIG. 18G shows quantification of VCN-01 genomes in subcutaneous (s.c.) retinoblastomas (Y79 and RBT-7) 5 days after local injection of VCN-01 and subsequent treatment with saline, TPT (0.6 mg/kg, daily for five consecutively days), CBP (40 mg/kg, one single dose at day 1) or HU (200 mg/kg, daily for five consecutively days). Dots are values from individual tumors. FIG. 18H shows mRNA expression of recombinant hyaluronidase PH20 (SPAM1 gene) in s.c. retinoblastomas (Y79 and RBT-7) 5 days after local injection of VCN-01 and subsequent treatment with TPT or CBP. FIG. 18I shows immunostaining of E1A in s.c. Y79 xenografts 5 days after local injection of VCN-01 and subsequent treatment with TPT, CBP or HU. FIG. 18J immunoblotting of E1A in s.c. Y79 xenografts 5 or 15 days after local injection of VCN-01 and subsequent treatment with TPT, CBP or HU. Each sample corresponds to one individual tumor.

FIGS. 19A-B show adenoviral transduction and replication in retinoblastoma cells exposed to topotecan. FIG. 19A shows proportion (%) of GFP⁺RBT-5 and RBT-7 cells pretreated with AdTLRGK for 24 h (50 MOI) and exposed for additional 24 h to culture medium (Medium), topotecan (TPT; 2 μM), carboplatin (CBP; 12.5 μM), melphalan (MEL; 12 μM) or hydroxyurea (HU; 100 μM). Dots are experimental replicates and bars are mean and SD. FIG. 19B shows quantification of VCN-01 genomes in subcutaneous retinoblastomas (Y79 and RBT-7) 15 days after local injection of VCN-01 and subsequent treatment with saline, TPT (0.6 mg/kg, daily for five consecutively days), or CBP (40 mg/kg, one single dose at day 1). Dots are values from individual tumors.

FIGS. 20A-I show activity of local VCN-01 and systemic topotecan in retinoblastoma xenografts. FIG. 20A shows ocular survival of eyes with Y79 xenografts treated with intravitreous VCN-01, standard-of-care chemotherapy (SoC), topotecan (TPT) or the combinations. FIG. 20B shows ocular survival of eyes with Y79 xenografts treated with topotecan, VCN-01 or the combination in the sequences topotecan first (TPT+VCN-01) or virus first (VCN-01+TPT). FIG. 20C shows ocular survival of eyes with RBT-7 xenografts treated with topotecan, VCN-01 or the combination. FIG. 20D shows representative image of a mouse with bilateral RBT-7 intraocular xenografts treated with systemic topotecan and one dose of VCN-01 into the left eye, and saline in the right eye. The image was obtained 30 days after treatment start. FIG. 20E shows representative H&E staining in a survivor eye (day 80) with an RBT-7 xenograft treated with VCN-01 and topotecan. FIG. 20F shows E1A immunostaining in an RBT-7 intraocular xenograft treated with VCN-01 and topotecan and enucleated at endpoint (65 days after VCN-01 injection). FIG. 20G shows ocular survival of eyes with RBT-2 xenografts treated with maximized dosing schedules including VCN-01 (two injections), topotecan (6 cycles) and the combination. FIG. 20H shows survival of mice bearing Y79 or RBT-7 s.c. xenografts treated with one intratumoral injection of VCN-01, systemic topotecan, or the combination. FIG. 20I shows antitumor activity of one intratumoral injection of VCN-01, systemic topotecan, or the combination in s.c. retinoblastomas Y79 and RBT-7. Graphs show growth of individual tumors.

FIG. 21 shows intratumoral accumulation of topotecan (TPT), lactone and total, at the steady state (i.e., at constant concentration in plasma), in orthotopic retinoblastoma xenografts (RBT-2) pre-treated with a local intraocular injection of adenovirus (VCN-01) or vehicle solution (Vehicle). Dots are values obtained from individual tumors and lines are means and SD.

FIGS. 22A-J show activity of local VCN-01 and systemic topotecan in CNS-disseminated retinoblastoma. FIG. 22A shows engraftment of RBT-7 cells in the mouse brain at endpoint. The whole brain is stained with hematoxylin and eosin (H&E). Cancer cells in the high magnification images are stained with the anti-human nuclei antibody, in brown. FIG. 22B shows engraftment of RBT-7 cells in the mouse meninges surrounding the spinal cord, at endpoint (H&E and anti-human nuclei). FIG. 22C shows survival of mice bearing RBT-7 intracerebral tumors treated with standard of care chemotherapy (SoC), topotecan (TPT) alone, VCN-01 alone, or the combination of VCN-01 and topotecan. FIG. 22D shows images of mice receiving VCN-01 alone or with topotecan 25 days after tumor inoculation (i.e., 17 days after VCN-01 injection). Numbers on the mice are individual weights (g). FIG. 22E shows immunostaining of human cells (anti-human nuclei, in brown) in mice sacrificed at day 26 after tumor inoculation. FIG. 22F shows tumor burden (expression of the gene CRX) in brain homogenates at end of treatment (5 days after the intraventricular injection of VCN-01; i.e., at day 12 after tumor inoculation). Dots are data from individual brains and bars are mean and SD. FIG. 22G shows expression of CDKN1A and E2F1 in brain homogenates at end of treatment. Dots are individual brains, among which the highest values are identified with the mouse number. FIG. 22H shows quantification of VCN-01 genomes at end of treatment in brain homogenates. FIG. 22I shows quantification of mRNA of the human hyaluronidase gene at end of treatment in brain homogenates. FIG. 22J shows immunoblotting of the adenoviral hexon in brain homogenates obtained at end of treatment. GAPDH was the loading control. Samples are numbered with the identification of the mice.

FIGS. 23A-C show toxicity and infection of the treatment with intraventricular VCN-01 in mice with CNS-disseminated retinoblastoma (RBT-7). FIG. 23A shows individual weights of mice bearing RBT-7 intracerebral tumors treated with one intraventricular injection of VCN-01, standard of care chemotherapy (SoC), topotecan (TPT) alone, VCN-01 alone, or the combination of VCN-01 and TPT. Control mice were treated with one intraventricular injection of vehicle. FIG. 23B shows immunostaining of the leukocyte marker CD45 in the brain of one mouse bearing an RBT-7 intracerebral xenograft, treated with the combination of intraventricular VCN-01 and SoC and sacrificed due to acute weight loss 9 days after VCN-01 inoculation. The high magnification image corresponds to an area with profuse infiltration of CD45+ cells. FIG. 23C shows immunostaining of E1A in the brain of mice bearing RBT-7 xenografts, treated with one intraventricular dose of VCN-01, alone (VCN-01) or in combination with TPT (VCN-01+TPT). The mice were sacrificed and samples were obtained at the end of treatments. High magnification images correspond to areas with tumor.

FIGS. 24A-G show activity of local VCN-01 and systemic topotecan in Ewing sarcoma and neuroblastoma PDX. FIG. 24A shows gene expression of E2F1, CXADR and ITGA5 in retinoblastoma, neuroblastoma, Ewing sarcoma and their control tissues fetal retina, pediatric brain and muscle, respectively. Each tumor was compared to its control (Mann-Whitney test). FIG. 24B shows immunostaining of E2F-1 in patient biopsies of retinoblastoma (positive control), Ewing sarcoma and neuroblastoma corresponding to HSJD patients from which the PDX ES-033 and NB-005 were established. FIG. 24C shows immunoblotting of E2F-1 and cyclin E1 in NB-005 and ES-003 cells treated for up to 48 h with topotecan. GAPDH was the loading control. FIG. 24D shows survival of mice bearing neuroblastoma treated with local VCN-01, topotecan (TPT), irinotecan (IRN) or the combinations. FIG. 24E shows survival of the PDX ES-033 treated with local VCN-01, TPT or their combination. FIG. 24F shows tumor volumes (individual values) of neuroblastoma PDX treated with local VCN-01, TPT, IRN or the combinations. FIG. 24G shows tumor volumes (individual values) of the Ewing sarcoma PDX treated with local VCN-01, TPT or the combination.

FIG. 25 shows antiproliferative activity of VCN-01 (6 days treatment) against PDX-derived primary cells in culture. Values are means and SD of six replicates. Lines are the best fitting curves built using the least square regression method of GraphPad. Cell viability is the relative percentage of the assay signal of treated cells, compared to control untreated cells, which was set as 100%.

### DETAILED DESCRIPTION

### Overview

The present disclosure is based, inter alia, on the discovery that it is possible to use genetically modified oncolytic adenoviruses in combination with a topoisomerase I inhibitor or prodrug thereof, e.g., topotecan, SN-38 or irinotecan to treat retinoblastoma, Ewing sarcoma, or neuroblastoma. This discovery is surprising for various reasons, including, without limitation, the fact that topotecan and irinotecan, a prodrug of the topoisomerase I inhibitor SN-38, but not other chemotherapies such as carboplatin or melphalan, enhanced viral activity in retinoblastoma cells therein of the aforementioned modified oncolytic adenovirus.

The inventors of the present disclosure have surprisingly found that oncolytic adenoviruses described herein, i.e., oncolytic adenoviruses comprising a sequence encoding a hyaluronidase enzyme inserted into its genome and replication machinery specific for tumor cells, can be used in combination with a topoisomerase I inhibitor or prodrug thereof, e.g., topotecan or a prodrug of a topoisomerase I inhibitor (e.g. irinotecan, which is converted to SN-38 in vivo), to effectively treat retinoblastoma, Ewing sarcoma, or neuroblastoma. Without wishing to be bound by theory, it is believed that administration of the topotecan, SN-38 or irinotecan may sensitize the retinoblastoma, Ewing sarcoma, or neuroblastoma to the described oncolytic adenoviruses and enhance the replication of the described oncolytic virus in these tumors.

### Treatment of Retinoblastoma

In aspects, the present disclosure provides methods of treating retinoblastoma in a patient in need thereof, comprising administering to the patient (i) an oncolytic adenovirus comprising replication machinery specific for tumor cells and, optionally, a polynucleotide sequence encoding a hyaluronidase enzyme inserted into its genome, and (ii) a topoisomerase I inhibitor or a prodrug thereof, e.g., topotecan, SN-38, or irinotecan.

In aspects, the present disclosure provides methods of preventing, removing or reducing metastases, secondary malignancies and or trilateral retinoblastoma associated with retinoblastoma, in a patient in need thereof, comprising administering to the patient (i) an oncolytic adenovirus comprising replication machinery specific for tumor cells and, optionally, a polynucleotide sequence encoding a hyaluronidase enzyme inserted into its genome and (ii) a topoisomerase I inhibitor, or a prodrug thereof, e.g., topotecan, SN-38, or irinotecan.

Retinoblastoma is the most common type of eye cancer in children. It is usually found in children under the age of two. When the eyes are developing, they have progenitor cells called retinoblasts. These cells divide into new cells and fill the part of the eye that will become the retina.

Non-hereditary retinoblastoma, also called sporadic retinoblastoma, happens by chance. About 60% of children with retinoblastoma have non-hereditary retinoblastoma. Children are born with two normal copies of the RB1 gene. A mutation of both copies of the RB1 gene in a retinoblast causes a retinoblastoma tumor to form in the eye. Children with non-hereditary retinoblastoma only develop a tumor in one eye, called unilateral retinoblastoma. They will not usually pass the RB1 mutation on to their future children.

Hereditary retinoblastoma is passed on from a parent to a child. About 40% of children with retinoblastoma have hereditary retinoblastoma. Hereditary retinoblastoma can be familial or sporadic. In familial hereditary retinoblastoma, also called familial retinoblastoma, a parent or other family member of the child has had retinoblastoma. In sporadic hereditary retinoblastoma, no one else in the family has a history of retinoblastoma. The RB1 gene mutation happens as a germline mutation in the egg or sperm before a baby is conceived and is passed on to the child. In all cases of hereditary retinoblastoma, a child is born with one copy of the RB1 mutation in all cells of the body. A mutation of the second copy of the RB1 gene then occurs in retinoblast cells and causes retinoblastoma to form. Children with hereditary retinoblastoma can have more than one tumor, and tumors can occur in one eye or both eyes, called bilateral retinoblastoma. Children with hereditary retinoblastoma can pass the RB1 mutation on to their future children. They also have a greater risk of developing other cancers.

In embodiments, the retinoblastoma is non-hereditary retinoblastoma. In embodiments, the retinoblastoma is hereditary retinoblastoma.

Various staging systems are used for classifying retinoblastoma, based on how far the cancer system has spread. The prognosis for children with retinoblastoma depends, to some extent, on the cancer's stage. The stage is also an important factor in choosing treatment.

Retinoblastoma is often staged based on the results of eye exams, imaging tests, and any other relevant tests. When determining the treatment options, retinoblastomas are often divided into two main groups: (i) intraocular retinoblastoma, in which the cancer is still within the eye, and (ii) extraocular retinoblastoma, in which the cancer has spread outside the eye. Extraocular cancers can be divided further into orbital retinoblastomas, which have spread only to the eye socket, and metastatic retinoblastomas, which have spread to distant parts of the body, such as the brain or bone marrow.

In embodiments, the retinoblastoma is an intraocular retinoblastoma. In embodiments, the retinoblastoma is an extraocular retinoblastoma. In embodiments, the retinoblastoma is an orbital retinoblastoma. In embodiments, the retinoblastoma is a metastatic retinoblastoma.

In embodiments, the method prevents progression of an intraocular retinoblastoma to an extraocular retinoblastoma. In embodiments, the method prevents progression of an intraocular retinoblastoma to an orbital retinoblastoma. In embodiments, the method prevents progression of an intraocular retinoblastoma to a metastatic retinoblastoma. In embodiments, the method prevents progression of an orbital retinoblastoma to a metastatic retinoblastoma.

In the United States, most retinoblastomas are diagnosed before they have spread outside the eye, so staging systems that apply only to intraocular retinoblastoma are used most often in this country. There are two commonly used staging systems for intraocular retinoblastomas: the International Classification for Intraocular Retinoblastoma and the Reese-Ellsworth Staging System.

The International Classification for Intraocular Retinoblastoma divides intraocular retinoblastomas into five groups, A through E, based on the extent of the cancer and on the chances that the eye can be saved using current treatment options.
Group A: Small tumors (no more than 3 mm across) that are only in the retina and are not near important structures such as the optic disc, i.e., where the optic nerve enters the retina, or the foveola, i.e., the center of vision.
Group B: All other tumors (either larger than 3 mm or close to the optic disc or foveola) that are still only in the retina.
Group C: Well-defined tumors with small amounts of spread under the retina, i.e., subretinal seeding or vitreous seeding.
Group D: Large or poorly defined tumors with widespread vitreous or subretinal seeding. The retina may have become detached from the back of the eye.
Group E: The tumor is very large, extends near the front of the eye, is bleeding or causing glaucoma, i.e., high pressure inside the eye, or has other features that mean there is almost no chance the eye can be saved.

In embodiments, the retinoblastoma is classified using the International Classification for Intraocular Retinoblastoma and is classified as Group A. In embodiments, the retinoblastoma is classified using the International Classification for Intraocular Retinoblastoma and is classified as Group B. In embodiments, the retinoblastoma is classified using the International Classification for Intraocular Retinoblastoma and is classified as Group C. In embodiments, the retinoblastoma is classified using the International Classification for Intraocular Retinoblastoma and is classified as Group D. In embodiments, the retinoblastoma is classified using the International Classification for Intraocular Retinoblastoma and is classified as Group E.

In embodiments, the method prevents a Group A retinoblastoma from progressing to a Group B retinoblastoma, a Group C retinoblastoma, a Group D retinoblastoma, or a Group E retinoblastoma. In embodiments, the method prevents a Group B retinoblastoma from progressing to a Group C retinoblastoma, a Group D retinoblastoma, or a Group E retinoblastoma. In embodiments, the method prevents a Group C retinoblastoma from progressing to a Group D retinoblastoma, or a Group E retinoblastoma. In embodiments, the method prevents a Group D retinoblastoma from progressing to a Group E retinoblastoma.

The Reese-Ellsworth staging system divides intraocular retinoblastoma into five groups. The higher the group number, from 1 to 5, the lower the chance of controlling the retinoblastoma or of saving the eye or any useful vision.
Group 1 (very favorable for saving or preserving the eye)—1A: one tumor, smaller than 4 disc diameters (DD), at or behind the equator; 1B: multiple tumors smaller than 4 DD, all at or behind the equator.
Group 2 (favorable for saving or preserving the eye)—2A: one tumor, 4 to 10 DD, at or behind the equator; 2B: multiple tumors, with at least one 4 to 10 DD, and all at or behind the equator.
Group 3 (doubtful for saving or preserving the eye)—3A: any tumor in front of the equator; 3B: one tumor, larger than 10 DD, behind the equator.
Group 4 (unfavorable for saving (or preserving) the eye)—4A: multiple tumors, some larger than 10 DD; 4B: any tumor extending toward the front of the eye to the ora serrata (front edge of the retina).
Group 5 (very unfavorable for saving (or preserving) the eye)—5A: tumors involving more than half of the retina; 5B: vitreous seeding.

In embodiments, the retinoblastoma is classified using the Reese-Ellsworth system and is classified as Group 1A. In embodiments, the retinoblastoma is classified using the Reese-Ellsworth system and is classified as Group 1B. In embodiments, the retinoblastoma is classified using the Reese-Ellsworth system and is classified as Group 2A. In embodiments, the retinoblastoma is classified using the Reese-Ellsworth system and is classified as Group 2B. In embodiments, the retinoblastoma is classified using the Reese-Ellsworth system and is classified as Group 3A. In embodiments, the retinoblastoma is classified using the Reese-Ellsworth system and is classified as Group 3B. In embodiments, the retinoblastoma is classified using the Reese-Ellsworth system and is classified as Group 4A. In embodiments, the retinoblastoma is classified using the Reese-Ellsworth system and is classified as Group 4B. In embodiments, the retinoblastoma is classified using the Reese-Ellsworth system and is classified as Group 5A. In embodiments, the retinoblastoma is classified using the Reese-Ellsworth system and is classified as Group 5B.

In embodiments, the method prevents a Group 1A retinoblastoma from progressing to a Group 1B retinoblastoma, a Group 2A retinoblastoma, a Group 2B retinoblastoma, a Group 3A retinoblastoma, a Group 3B retinoblastoma, a Group 4A retinoblastoma, a Group 4B retinoblastoma, a Group 5A retinoblastoma, or a Group 5B retinoblastoma. In embodiments, the method prevents a Group 1B retinoblastoma from progressing to a Group 2A retinoblastoma, a Group 2B retinoblastoma, a Group 3A retinoblastoma, a Group 3B retinoblastoma, a Group 4A retinoblastoma, a Group 4B retinoblastoma, a Group 5A retinoblastoma, or a Group 5B retinoblastoma. In embodiments, the method prevents a Group 2A retinoblastoma from progressing to a Group 2B retinoblastoma, a Group 3A retinoblastoma, a Group 3B retinoblastoma, a Group 4A retinoblastoma, a Group 4B retinoblastoma, a Group 5A retinoblastoma, or a Group 5B retinoblastoma. In embodiments, the method prevents a Group 2B retinoblastoma from progressing to a Group 3A retinoblastoma, a Group 3B retinoblastoma, a Group 4A retinoblastoma, a Group 4B retinoblastoma, a Group 5A retinoblastoma, or a Group 5B retinoblastoma. In embodiments, the method prevents a Group 3A retinoblastoma from progressing to a Group 3B retinoblastoma, a Group 4A retinoblastoma, a Group 4B retinoblastoma, a Group 5A retinoblastoma, or a Group 5B retinoblastoma. In embodiments, the method prevents a Group 3B retinoblastoma from progressing to a Group 4A retinoblastoma, a Group 4B retinoblastoma, a Group 5A retinoblastoma, or a Group 5B retinoblastoma. In embodiments, the method prevents a Group 4A retinoblastoma from progressing to a Group 4B retinoblastoma, a Group 5A retinoblastoma, or a Group 5B retinoblastoma. In embodiments, the method prevents a Group 4B retinoblastoma from progressing to a Group 5A retinoblastoma, or a Group 5B retinoblastoma. In embodiments, the method prevents a Group 5A retinoblastoma from progressing to a Group 5B retinoblastoma.

Other staging systems that include both intraocular retinoblastomas and those that have spread beyond the eye (extraocular retinoblastomas) may be used by some doctors. These can be especially useful in countries where these cancers are more likely to have spread by the time they are found. For example, the American Joint Commission on Cancer (AJCC) staging system considers four key pieces of information:
T: The size of the primary tumor and how far it has grown within and outside of the eye.
N: Whether the cancer has reached nearby lymph nodes in the head or neck.
M: Whether the cancer has metastasized to distant parts of the body, such as the bone marrow, brain, skull, or long bones.
H: Whether the child has the heritable form of retinoblastoma

This system is used to describe the extent of retinoblastomas in detail, particularly for those that have spread outside the eye.

In embodiments, the retinoblastoma is classified using the AJCC staging system.

In embodiments, the method described herein spares the patient from requiring enucleation.

### Treatment of Ewing Sarcoma

In aspects, the present disclosure provides methods of treating Ewing sarcoma in a patient in need thereof, comprising administering to the patient (i) an oncolytic adenovirus comprising replication machinery specific for tumor cells and, optionally, a polynucleotide sequence encoding a hyaluronidase enzyme inserted into its genome and replication machinery specific for tumor cells, and (ii) a topoisomerase I inhibitor or prodrug thereof, e.g., topotecan, SN-38, or irinotecan.

In aspects, the present disclosure provides methods of treating Ewing sarcoma in a patient in need thereof, comprising administering to the patient (i) an oncolytic adenovirus comprising replication machinery specific for tumor cells and a polynucleotide sequence encoding a hyaluronidase enzyme inserted into its genome and replication machinery specific for tumor cells, and (ii) a topoisomerase I inhibitor or prodrug thereof, e.g., topotecan, SN-38, or irinotecan.

Ewing tumors (also known as Ewing sarcomas) are a group of cancers that start in the bones or nearby soft tissues and share some common features. These tumors can develop in people of any age, but they are most common in older children and teens.

The main types of Ewing tumors are Ewing sarcoma of bone, Extraosseous Ewing tumor (EOE), and Peripheral primitive neuroectodermal tumor (PPNET). Ewing sarcoma of bone is the most common and is Ewing sarcoma that starts in a bone. EOE tumors start in soft tissues around bones, but they look and act very much like Ewing sarcomas in bones. They are also known as extraskeletal Ewing sarcomas. PPNET is a rare childhood cancer that also starts in bone or soft tissue and shares many features with Ewing sarcoma of bone and EOE. Peripheral PNETs that start in the chest wall are known as Askin tumors.

Most Ewing tumors occur in the bones. The most common sites are the pelvis (hip bones), the chest wall (such as the ribs or shoulder blades), and the legs (mainly in the middle of the long bones). Extraosseous Ewing tumors can occur almost anywhere.

In embodiments, the Ewing sarcoma is Ewing sarcoma of bone. In embodiments, the Ewing sarcoma is EOE. In embodiments, the Ewing sarcoma is PPNET.

Treatment of a Ewing sarcoma is based mainly on where it is in the body and how far it has spread when it is first found and can generally be classified as localized, metastatic, or recurring Ewing sarcoma.

A localized Ewing tumor is one that still appears to be confined to the area where it started (and maybe also to nearby tissues such as muscle or tendons), based on imaging test and biopsy results. But even people with localized Ewing tumors often still have small areas of cancer in other parts of the body that cannot be seen with imaging tests but can grow. Thus, chemotherapy is an important part of treatment for localized Ewing tumors to prevent their spread.

Once the Ewing tumor has been diagnosed and staged, the first treatment is chemotherapy. It is called neoadjuvant chemotherapy because it is given before any surgery or radiation therapy. In the United States, patients are given a chemotherapy regimen known as VDC/IE (or VAC/IE), which is a combination of vincristine, doxorubicin (Adriamycin), and cyclophosphamide, alternated with ifosfamide and etoposide, although other combinations of the same drugs are also effective.

After at least 9 weeks of chemotherapy, imaging tests such as CT, MRI, PET, or bone scans are done to see if the tumor is shrinking, or at least is not growing, and if it can be surgically removed. If so, surgery is done at this point, and the surgery specimen is analyzed by a pathologist. If cancer cells are found at or near the edges of the surgery specimen, meaning cancer cells may have been left behind, radiation therapy and chemotherapy for several months are used. If there are no cancer cells at or near the edges of the surgery specimen, chemotherapy can be used without radiation therapy.

If surgery is not an option after the initial chemotherapy, but the tumor is not growing, radiation therapy along with chemotherapy is usually the next treatment given. In some cases this might shrink the tumor enough so that surgery can then be done. This would then be followed by more chemotherapy, possibly with more radiation as well. In other cases where surgery is still not an option, radiation therapy and chemotherapy are the main treatments.

If the Ewing tumor continues to grow despite the initial chemotherapy, a second type of chemotherapy may be tried. Surgery or radiation therapy may also be tried to help keep the tumor under control. This may be followed by more chemotherapy.

Patients who clearly have metastatic disease when they are first diagnosed are harder to treat than patients with localized disease. The outlook tends to be better when the cancer has only spread to the lungs, as opposed to when the cancer has spread to other bones or to the bone marrow.

Treating metastatic disease is similar in many ways to treating localized disease. Chemotherapy is the first treatment, but it often requires a more intense regimen than would be used if the cancer was localized. After a few months, tests such as CT or MRI scans, bone or PET scans, and/or bone marrow biopsies are done to see how the cancer has responded to treatment.

If the cancer remains in only a few small areas after chemotherapy, the primary tumor and all known areas of metastases may be removed with surgery at this point. Other approaches, such as surgery plus radiation therapy (before and/or after surgery) or just radiation therapy to all known metastatic sites, might also be options. During and after these treatments, chemotherapy is given for several months as well.

Another treatment that is being studied is very intensive chemotherapy followed by a stem cell transplant to try to improve the outcome for these patients.

Recurrence of Ewing tumors after treatment is less likely now than in the past, but it can happen. If the tumor does come back, treatment depends on a number of factors, including: the size and location of the tumor, if it has spread to different parts of the body, what types of treatment were used before, and how long it has been since treatment.

Chemotherapy, surgery, radiation therapy, or some combination of these may be used to treat recurrent tumors, depending on the situation.

Other treatments that are being studied include high-dose chemotherapy followed by a stem cell transplant, and targeted drugs and immune therapies.

In embodiments, the Ewing sarcoma is a localized Ewing tumor. In embodiments, the Ewing sarcoma is a metastatic Ewing tumor. In embodiments, the Ewing sarcoma is a recurrent Ewing tumor.

In embodiments, the method prevents progression of a localized Ewing tumor to a metastatic Ewing tumor or a recurrent Ewing tumor. In embodiments, the method prevents progression of a metastatic Ewing tumor to a recurrent Ewing tumor.

The American Joint Committee on Cancer (AJCC) uses one system to describe all bone cancers, including Ewing tumors that start in bone. The AJCC stages EOE tumors, which do not start in bones, like soft tissue sarcomas. The AJCC staging system for bone cancers and for soft tissue sarcomas is based on 4 key pieces of information:
T describes the size of the primary tumor and whether it appears in different areas of the bone.
N describes the extent of spread to regional lymph nodes. Bone tumors rarely spread to the lymph nodes.
M indicates whether the cancer has metastasized to other organs of the body. (The most common sites of spread are to the lungs or other bones.)
G stands for the grade of the tumor, which describes how the cells from biopsy samples look. Low-grade tumor cells look more like normal cells and are less likely to grow and spread quickly, while high-grade tumor cells look more abnormal.

Once the T, N, M, and G categories have been determined, the information is combined and expressed as an overall stage. The process of assigning a stage number is called stage grouping. The stages are described in Roman numerals from I to IV, and are sometimes divided further.

Stage I is not used for Ewing sarcoma. This is because all Ewing sarcomas are high grade (G2 or 3). Stage I is used for other kinds of bone cancer.

Stage II is divided into these 2 groups:
Stage IIA. The tumor is no more than 8 cm across and is high grade. The cancer has not spread to nearby lymph nodes or organs in other parts of the body.
Stage IIB. The tumor is more than 8 cm across and is high grade. The cancer has not spread to nearby lymph nodes or organs in other parts of the body.

Stage III means the tumor is in more than 1 spot in the same bone and is high grade. The cancer has not spread to nearby lymph nodes or organs in other parts of the body.

Stage IV is divided into these 2 groups:
Stage IVA. The cancer has spread to the lungs, but not to lymph nodes or organs in other parts of the body. It can be any size or grade.
Stage IVB is either of these:
   The cancer has spread to nearby lymph nodes. It may or may not have spread to organs in other parts of the body. It can be any size or grade.
   The cancer has spread to organs in other parts of the body, but not the lungs. It can be any size or grade.

In embodiments, the Ewing sarcoma is stage II. In embodiments, the Ewing sarcoma is stage III. In embodiments, the Ewing sarcoma is stage IV. In embodiments, the Ewing sarcoma is stage IIA. In embodiments, the Ewing sarcoma is stage IIB. In embodiments, the Ewing sarcoma is stage IIIA. In embodiments, the Ewing sarcoma is stage IIIB. In embodiments, the Ewing sarcoma is stage IVA. In embodiments, the Ewing sarcoma is stage IVB.

In embodiments, the method prevents progression of a stage II Ewing sarcoma to a stage III or a stage IV Ewing sarcoma. In embodiments, the method prevents progression of a stage III Ewing sarcoma to a stage IV Ewing sarcoma. In embodiments, the method prevents progression of a stage IIA Ewing sarcoma to a stage IIB, stage IIIA, stage IIIB, stage IVA, or stage IVB Ewing sarcoma. In embodiments, the method prevents progression of a stage IIB Ewing sarcoma to a stage IIIA, stage IIIB, stage IVA, or stage IVB Ewing sarcoma. In embodiments, the method prevents progression of a stage IIIA Ewing sarcoma to a stage IIIB, stage IVA, or stage IVB Ewing sarcoma. In embodiments, the method prevents progression of a stage IIIB Ewing sarcoma to a stage IVA or stage IVB Ewing sarcoma. In embodiments, the method prevents progression of a stage IVA Ewing sarcoma to a stage IVB Ewing sarcoma.

### Treatment of Neuroblastoma

In aspects, the present disclosure provides methods of treating neuroblastoma in a patient in need thereof, comprising administering to the patient (i) an oncolytic adenovirus comprising replication machinery specific for tumor cells and, optionally, a polynucleotide sequence encoding a hyaluronidase enzyme inserted into its genome and replication machinery specific for tumor cells, and (ii) a topoisomerase I inhibitor or prodrug thereof, e.g., topotecan, SN-38, or irinotecan.

In aspects, the present disclosure provides methods of treating neuroblastoma in a patient in need thereof, comprising administering to the patient (i) an oncolytic adenovirus comprising replication machinery specific for tumor cells and a polynucleotide sequence encoding a hyaluronidase enzyme inserted into its genome and replication machinery specific for tumor cells, and (ii) a topoisomerase I inhibitor or prodrug thereof, e.g., topotecan, SN-38, or irinotecan.

Neuroblastoma is a cancer that starts in neuroblasts of the sympathetic nervous system. This type of cancer occurs most often in infants and young children.

Most neuroblastomas begin in the abdomen, either in an adrenal gland or in sympathetic nerve ganglia. Most of the rest start in sympathetic ganglia near the spine in the chest or neck, or in the pelvis. Rarely, a neuroblastoma has spread so widely by the time it is found that doctors cannot tell exactly where it started. Some neuroblastomas grow and spread quickly, while others grow slowly. Sometimes, in very young children, the cancer cells die for no reason and the tumor goes away on its own. In other cases, the cells sometimes mature on their own into normal ganglion cells and stop dividing, making the tumor a benign ganglioneuroma.

There are two systems used for neuroblastoma staging. The main difference between them is whether the staging system can be used to help determine a child's risk group before treatment has started. The International Neuroblastoma Risk Group Staging System (INRGSS) uses results from imaging tests (such as CT or MRI and MIBG scans) to help decide the stage. The INRGSS stage can be determined before treatment has started. The International Neuroblastoma Staging System (INSS) is based on the results from the surgery to remove a child's tumor instead of imaging tests.

The INRGSS was developed to help determine a child's stage and risk group before treatment has started. INRGSS uses imaging tests (usually a CT or MRI scan, and an MIBG scan), as well as exams and biopsies to help define the stage. The stage can then be used to help predict how resectable the tumor is. The INRGSS uses image-defined risk factors (IDRFs), which are factors seen on imaging tests that might mean the tumor will be harder to remove. This includes things like the tumor growing into a nearby vital organ or growing around important blood vessels. The INRGSS divides neuroblastomas into 4 stages:
L1: The tumor has not spread from where it started and has not grown into vital structures as defined by the list of IDRFs. It is confined to one area of the body, such as the neck, chest, or abdomen.
L2: The tumor has not spread far from where it started (for example, it may have grown from the left side of the abdomen into the left side of the chest), but it has at least one IDRF.
M: The tumor has metastasized to a distant part of the body (except tumors that are stage MS).
MS: Metastatic disease in children younger than 18 months, with cancer spread only to skin, liver, and/or bone marrow.

In embodiments, the neuroblastoma is a stage L1 neuroblsatoma. In embodiments, the neuroblastoma is a stage L2 neuroblsatoma. In embodiments, the neuroblastoma is a stage M neuroblastoma. In embodiments, the neuroblastoma is a stage MS neuroblastoma.

In embodiments, the method prevents progression of a stage L1 neuroblastoma to a stage L2, M, or MS neuroblastoma. In embodiments, the method prevents progression of a stage L2 neuroblastoma to a stage M or MS neuroblastoma.

The INSS takes into account the results of surgery to remove the tumor. It cannot help doctors determine a stage before any treatment has started, so it does not work as well for children who do not need or cannot have surgery. The stages are:
Stage 1: The cancer is still in the area where it started. It is on one side of the body (right or left). All visible tumor has been removed completely by surgery (although looking at the tumor's edges under the microscope after surgery may show some cancer cells). Lymph nodes near the tumor are free of cancer (although nodes enclosed within the tumor may contain neuroblastoma cells).
Stage 2A: The cancer is still in the area where it started and on one side of the body, but not all of the visible tumor could be removed by surgery. Lymph nodes near the tumor are free of cancer (although nodes enclosed within the tumor may contain neuroblastoma cells).
Stage 2B: The cancer is on one side of the body, and it may or may not have been removed completely by surgery. Nearby lymph nodes outside the tumor contain neuroblastoma cells, but the cancer has not spread to lymph nodes on the other side of the body or elsewhere.
Stage 3: The cancer has not spread to distant parts of the body, but one of the following is true:
   The cancer cannot be removed completely by surgery, and it has crossed the midline (defined as the spine) to the other side of the body. It may or may not have spread to nearby lymph nodes.
   The cancer is still in the area where it started and is on one side of the body. It has spread to lymph nodes that are relatively nearby but on the other side of the body.
   The cancer is in the middle of the body and is growing toward both sides (either directly or by spreading to nearby lymph nodes).
Stage 4: The cancer has spread to distant parts of the body such as distant lymph nodes, bones, liver, skin, bone marrow, or other organs (but the child does not meet the criteria for stage 4S).
Stage 4S (also called “special” neuroblastoma): The child is younger than 1 year old. The cancer is on one side of the body. It might have spread to lymph nodes on the same side of the body but not to nodes on the other side. The neuroblastoma has spread to the liver, skin, and/or the bone marrow. However, no more than 10% of marrow cells are cancer cells, and imaging tests such as an MIBG scan do not show cancer in the bone marrow.
Recurrent: While not a formal part of the staging system, this term is used to describe cancer that has come back after it has been treated. The cancer might come back in the area where it first started or in another part of the body.

In embodiments, the neuroblastoma is a stage 1 neuroblastoma. In embodiments, the neuroblastoma is a stage 2A neuroblastoma. In embodiments, the neuroblastoma is a stage 2B neuroblastoma. In embodiments, the neuroblastoma is a stage 3 neuroblastoma. In embodiments, the neuroblastoma is a stage 4 neuroblastoma. In embodiments, the neuroblastoma is a stage 4S neuroblastoma. In embodiments, the neuroblastoma is a recurrent neuroblastoma.

In embodiments, the method treats a stage 1 neuroblastoma. In embodiments, the method treats a stage 2A neuroblastoma. In embodiments, the method treats a stage 2B neuroblastoma. In embodiments, the method treats a stage 3 neuroblastoma. In embodiments, the method treats a stage 4 neuroblastoma. In embodiments, the method treats a stage 4S neuroblastoma. In embodiments, the method treats a recurrent neuroblastoma.

Prognostic markers are features that help predict whether the child's prognosis is better or worse than would be predicted by the stage alone. Many of these prognostic markers are used along with a child's stage to assign their risk group:
Age: Younger children (under 12-18 months) are more likely to have a better outcome than older children.
Tumor histology: Tumors that contain more normal-looking cells and tissues tend to have a better prognosis and are said to have a favorable histology. Tumors whose cells and tissues look more abnormal under a microscope tend to have a poorer prognosis and are said to have an unfavorable histology.
DNA ploidy: Neuroblastoma cells with about the same amount of DNA as normal cells (a DNA index of 1) are classified as diploid. Cells with increased amounts of DNA (a DNA index higher than 1) are termed hyperdiploid. Neuroblastoma cells with more DNA are associated with a better prognosis, particularly for children under 2 years of age. DNA ploidy is not as useful for understanding a prognosis in older children.
MYCN gene amplifications: MYCN is a gene that normally helps regulate cell growth. Changes in the MYCN gene can turn it into an oncogene. Neuroblastomas with amplification of the MYCN oncogene tend to grow quickly and can be harder to treat.
Chromosome changes: Tumor cells that are missing certain parts of chromosomes 1 or 11 (known as 1p deletions or 11q deletions) may predict a less favorable prognosis. Having an extra part of chromosome 17 (17q gain) is also linked with a worse prognosis.
Neurotrophin (nerve growth factor) receptors: These are substances on the surface of normal nerve cells and on some neuroblastoma cells. They normally allow the cells to recognize neurotrophins, which are hormone-like chemicals that help the nerve cells mature. Neuroblastomas that have more of certain neurotrophin receptors, especially the nerve growth factor receptor TrkA, may have a better prognosis.
Serum levels of certain substances can also be used to help predict prognosis. Ferritin is a chemical that is an important part of the body's normal iron metabolism, and patients with high ferritin levels tend to have a worse prognosis. Increased levels of lactate dehydrogenase (LDH) in the blood is also linked with a worse outlook in children with neuroblastoma.

### Co-Administration of Oncolytic Adenovirus and a Topoisomerase Inhibitor

In one aspect, the present disclosure provides methods for improving and/or increasing and/or enhancing antitumor efficacy in a patient in need thereof, comprising (a) administering to the patient (i) an oncolytic adenovirus comprising replication machinery specific for tumor cells and, optionally, a polynucleotide sequence encoding a hyaluronidase enzyme inserted into its genome and (ii) a topoisomerase I inhibitor or prodrug thereof, e.g., topotecan, SN-38, or irinotecan, and (b) observing improved antitumor efficacy compared to treatment with the topoisomerase I inhibitor or a prodrug thereof without the oncolytic adenovirus.

In one aspect, the present disclosure provides methods for improving and/or increasing and/or enhancing antitumor efficacy in a patient in need thereof, comprising (a) administering to the patient (i) an oncolytic adenovirus comprising replication machinery specific for tumor cells and a polynucleotide sequence encoding a hyaluronidase enzyme inserted into its genome and (ii) a topoisomerase I inhibitor or prodrug thereof, e.g., topotecan, SN-38, or irinotecan, and (b) observing improved antitumor efficacy compared to treatment with the topoisomerase I inhibitor or a prodrug thereof without the oncolytic adenovirus.

In embodiments, the oncolytic adenovirus, the topoisomerase I inhibitor or prodrug thereof, e.g., topotecan, SN-38, or irinotecan, of the present disclosure are co-administered. In embodiments, the co-administration can occur simultaneously or sequentially.

In embodiments, the oncolytic adenovirus and the topoisomerase I inhibitor or prodrug thereof, e.g., topotecan, SN-38, or irinotecan, are administered to a subject simultaneously. The term “simultaneously” as used herein, means that the oncolytic adenovirus and the topoisomerase I inhibitor or prodrug thereof, e.g., topotecan, SN-38, or irinotecan, are administered with a time separation of no more than about 60 minutes, such as no more than about 30 minutes, no more than about 20 minutes, no more than about 10 minutes, no more than about 5 minutes, or no more than about 1 minute. Administration of the oncolytic adenovirus and the topoisomerase I inhibitor or prodrug thereof, e.g., topotecan, SN-38, or irinotecan, can be by simultaneous administration of a single formulation (e.g., a formulation comprising the oncolytic adenovirus and the topoisomerase I inhibitor or prodrug thereof, e.g., topotecan, SN-38, or irinotecan) or of separate formulations (e.g., a first formulation including the oncolytic adenovirus and a second formulation including the topoisomerase I inhibitor or prodrug thereof, e.g., topotecan, SN-38, or irinotecan).

In embodiments, the oncolytic adenovirus and the topoisomerase I inhibitor or prodrug thereof, e.g., topotecan, SN-38, or irinotecan, are administered to a subject simultaneously but the release of the oncolytic adenovirus and the topoisomerase I inhibitor or prodrug thereof, e.g., topotecan, SN-38, or irinotecan, from their respective dosage forms (or single unit dosage form if co-formulated) may occur sequentially.

Co-administration does not require the oncolytic adenovirus and the topoisomerase I inhibitor or prodrug thereof, e.g., topotecan, SN-38, or irinotecan, to be administered simultaneously, if, for example, the timing of their administration is such that the pharmacological activities of the oncolytic adenovirus and the topoisomerase I inhibitor or prodrug thereof, e.g., topotecan, SN-38, or irinotecan, overlap in time. For example, the oncolytic adenovirus and the topoisomerase I inhibitor or prodrug thereof, e.g., topotecan, SN-38, or irinotecan, can be administered sequentially or contemporaneously. The terms “sequentially” and “contemporaneously” as used herein mean that the oncolytic adenovirus and the topoisomerase I inhibitor or prodrug thereof, e.g., topotecan, SN-38, or irinotecan, are administered with a time separation of more than about 60 minutes. For example, the time between the sequential administration of the oncolytic adenovirus and the topoisomerase I inhibitor or prodrug thereof, e.g., topotecan, SN-38, or irinotecan, can be more than about 60 minutes, more than about 2 hours, more than about 5 hours, more than about 10 hours, more than about 1 day, more than about 2 days, more than about 3 days, or more than about 1 week apart. The administration times will depend on the rates of metabolism, excretion, and/or the pharmacodynamic activity of the oncolytic adenovirus and the topoisomerase I inhibitor or prodrug thereof, e.g., topotecan, SN-38, or irinotecan, being administered. Either the oncolytic adenovirus or the topoisomerase I inhibitor or prodrug thereof, e.g., topotecan or SN-38, may be administered first.

In embodiments, the oncolytic adenovirus is administered first and the topoisomerase I inhibitor or prodrug thereof, e.g., topotecan, SN-38, or irinotecan, is administered about 60 minutes, about 2 hours, about 3 hours, about 4 hours, about 5 hours, about 6 hours, about 7 hours, about 8 hours, about 9 hours, about 10 hours, about 11 hours, about 12 hours, about 1 day, about 2 days, about 3 days, about 4 days, about 5 days, about 6 days, about 7 days, about 8 days, about 9 days, about 10 days, about 11 days, about 12 days, about 13 days, about 2 weeks, about 3 weeks, about 4 weeks, about 5 weeks, about 6 weeks, about 7 weeks, about 8 weeks, about 9 weeks, about 10 weeks, about 11 weeks, or about 12 weeks later.

In embodiments, co-administration also does not require the oncolytic adenovirus and the topoisomerase I inhibitor or prodrug thereof, e.g., topotecan, SN-38, or irinotecan, to be administered to the subject by the same route of administration. Rather, in embodiments, each therapeutic agent can be administered by any appropriate route, for example, intravenously, intravitreally, intrathecally, intraventricularly, intraocularly, intratumorally, intraperitoneally, orally, non-intravitreally, non-intrathecally, non-intraventricularly, non-intraocularly, non-intratumorally, non-intraperitoneally, or non-orally.

In embodiments, administration of the present compositions and formulations comprising the oncolytic adenovirus and/or topoisomerase I inhibitor or prodrug thereof, e.g., topotecan, SN-38, or irinotecan, may be combined with additional agents, such as one or more additional anti-tumor agents, including, but not limited to, one or more chemotherapy drugs, and/or one or more additional therapy conventionally used for treating retinoblastoma, Ewing sarcoma, or neuroblastoma. In embodiments, the additional agents are one or more of vincristine, carboplatin, etoposide, melphalan, doxorubicin, cyclophosphamide, ifosfamide, cisplatin, paclitaxel, docetaxel, oxaliplatin, and temozolamide. In embodiments, co-administration of the additional agent and the present compositions/formulations may be simultaneous or sequential. In embodiments, the additional agent can be included in the present compositions and formulations comprising the oncolytic adenovirus and/or topoisomerase I inhibitor or prodrug thereof, e.g., topotecan, SN-38, or irinotecan. In embodiments, the additional agent can be administered in a separate composition from the present compositions and formulations comprising the oncolytic adenovirus and/or topoisomerase I inhibitor or prodrug thereof, e.g., topotecan, SN-38, or irinotecan. In embodiments, the additional agent is administered at the same time as the present compositions and formulations comprising the oncolytic adenovirus and/or topoisomerase I inhibitor or prodrug thereof, e.g., topotecan, SN-38, or irinotecan. In embodiments, the additional agent is administered before the present compositions and formulations comprising the oncolytic adenovirus and/or topoisomerase I inhibitor or prodrug thereof, e.g., topotecan, SN-38, or irinotecan. In embodiments, the additional agent is administered after the present compositions and formulations comprising the oncolytic adenovirus and/or topoisomerase I inhibitor or prodrug thereof, e.g., topotecan, SN-38, or irinotecan. In embodiments, the present compositions and formulations comprising the oncolytic adenovirus and/or topoisomerase I inhibitor or prodrug thereof, e.g., topotecan, SN-38, or irinotecan, can be used alone in a therapeutic regimen for treating retinoblastoma, Ewing sarcoma, or neuroblastoma, i.e. without using other anti-tumor agents and/or other conventional therapies.

In embodiments, administration of the present compositions and formulations comprising the oncolytic adenovirus and/or topoisomerase I inhibitor or prodrug thereof, e.g., topotecan, SN-38, or irinotecan, may be combined with additional agents, such as one or more local and/or systemic corticosteroids. In embodiments, the one or more local and/or systemic corticosteroids are selected from hydrocortisone, prednisone, prednisolone, methylprednisolone, dexamethasone, betamethasone, alsosterone, budesonide, fluticasone, flunisolide, ciclesonide, mometasone, beclomethasone, triamcinolone, and tixocortol. In embodiments, the one or more local and/or systemic corticosteroids are selected from methylprednisolone, dexamethasone, betamethasone, and triamcinolone.

Further, the present compositions/formulations may comprise an additional agent (e.g. via co-formulation). For example, the additional agent and the oncolytic adenovirus and/or topoisomerase I inhibitor or prodrug thereof, e.g., topotecan, SN-38, or irinotecan, may be combined into a single formulation. Alternatively, the additional agent and the oncolytic adenovirus and/or topoisomerase I inhibitor or prodrug thereof, e.g., topotecan, SN-38, or irinotecan, may be formulated separately.

### Oncolytic Adenoviruses

The present disclosure is directed, in part, to pharmaceutical compositions, formulations, and uses of one or more oncolytic adenoviruses.

As used in the present document, “oncolytic adenovirus” and its plural refer to adenoviruses capable of replicating themselves or being replication-competent in the tumor cell. The oncolytic adenoviruses are differentiated from non-replicating adenoviruses because the latter are unable to replicate themselves in the target cell.

### Adenoviruses

In embodiments, oncolytic adenoviruses used in the present disclosure are oncolytic adenoviruses with replication machinery and a capsid that allows infection and replication in human cancer cells. In embodiments, the oncolytic adenovirus is generated from an adenovirus that infects humans. Examples of adenoviruses that infect humans include, but are not limited to, a human adenovirus of serotype 1 to 51, such as an adenovirus of serotype 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51 of human adenoviruses, or combinations thereof, for example a hybrid recombinant of two or more different serotypes of human adenoviruses.

The 51 serotypes of human adenoviruses that have been identified are classified into six groups, differentiated from A to F. Human adenovirus serotype 5 (Ad5), which belongs to group C, is a virus formed by an icosahedral protein capsid containing a 36-kilobase linear deoxyribonucleic acid (DNA). In adults, Ad5 infection is usually asymptomatic, and in children it causes common cold and conjunctivitis. Ad5 generally infects epithelial cells, the cells of the bronchial epithelium during a natural infection. It enters the cell by means of the interaction of the fiber, a viral protein that extends like an antenna from the twelve vertices of the capsid, with a cell protein involved in intercellular adhesion, called the Coxsackie-Adenovirus Receptor (CAR). When viral DNA reaches the interior of the nucleus, it starts systematically transcribing the early genes (E1 to E4) of the virus. The first viral genes to be expressed correspond to early region genes 1A (E1A). E1A binds to the cell protein of the retinoblastoma to release E2F and thus activate the transcription of other viral genes such as E2, E3, E4, and the cellular genes that activate the cell cycle. E1 B, for its part, binds to the p53 protein, to activate the cell cycle and prevent apoptosis of the infected cell. E2 encodes for virus replication proteins; E3 encodes proteins that inhibit the antiviral immune response; E4 encodes for proteins that transport viral RNA. Expression of the early genes leads to replication of the viral DNA, and once this is replicated, the major late promoter is activated, leading to expression of a messenger ribonucleic acid (RNA) transcript which, by cutting and differential splicing, generates all the RNAs that encode for the structural proteins that form the capsid.

In embodiments, an oncolytic adenovirus used in the present disclosure is generated from a human adenovirus serotype 5.

### Hyaluronidase

In embodiments, an oncolytic adenovirus used in the present disclosure comprises a sequence encoding a hyaluronidase enzyme inserted into its genome. In embodiments, an oncolytic adenovirus used in the present disclosure does not comprise a sequence encoding a hyaluronidase enzyme inserted into its genome.

Hyaluronidase enzymes are a family of enzymes responsible for degrading hyaluronic acid. In the human species, six genes that encode for hyaluronidase enzymes, having different properties and locations, have been located to date. The isoforms Hyal1 and Hyal2 are found in most tissues, Hyal1 being the predominant form in human plasma. Hyal3 is located in the bone marrow and testicles, but its function is not well characterized. The hyaluronidase enzyme PH20 is highly expressed in the testicles and is involved in the process by which the oocyte is fertilized by the spermatozoon. The hyaluronidase enzyme PH20 is anchored to the plasma membrane and the inner acrosomal membrane of the spermatozoa and gives the spermatozoon the ability to penetrate the extracellular matrix of the cumulus cells (which are rich in hyaluronic acid) and reach the zona pellucida of the oocyte. During the acrosome reaction, some of the hyaluronidase enzymes anchored in the spermatozoon membrane are processed enzymatically to give rise to a soluble form of the protein, which is released from the acrosomal membrane. The membrane protein PH20 is the only enzyme in the mammalian hyaluronidase family with activity at neutral pH.

Without wishing to be bound by theory, it is believed that expression of the hyaluronidase enzyme by an oncolytic adenovirus can help degrade the dense physical and immunosuppressive stroma barrier surrounding solid tumors, thereby ensuring greater tumor penetration by the oncolytic adenovirus and co-administered therapies. Without wishing to be bound by theory, this ensures that the adenovirus can reach and infect a larger number of tumor cells. Degrading the tumor stroma can also expose tumor neoantigens, thereby enabling an antitumor immune response by the patient's immune system.

In embodiments the hyaluronidase enzyme is a mammalian testicular hyaluronidase enzyme, optionally a human one (e.g., GenBank Gene ID: 6677), also known as SPAM1 or spermatozoon adhesion molecule 1 or PH20. In embodiments, the sequence of the hyaluronidase enzyme has the sequence corresponding to the enzyme having the membrane-binding carboxy-terminal domain deleted from it, such that the enzyme is soluble. When this carboxy-terminal domain is deleted, the resulting enzyme is secreted into the extracellular environment.

In embodiments, the sequence encoding the hyaluronidase enzyme inserted into an oncolytic adenovirus genome is SEQ ID NO: 1, from which nucleotides 1471 to 1527, corresponding to the carboxy-terminal domain, have been deleted.

Also, in embodiments, the hyaluronidase enzyme is a hyaluronidase variant. A hyaluronidase variant has at least one or more amino acid modifications, generally amino acid substitutions, as compared to the parental wild-type sequence. In embodiments, a hyaluronidase of the disclosure comprises an amino sequence having at least about 60% (e.g. about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, or about 99%) sequence identity with any of the sequences disclosed herein. In addition, in embodiments, hyaluronidase variants retain most or all of their biochemical activity, measured by any suitable method known in the art.

In embodiments, a hyaluronidase of the disclosure comprises an amino sequence having at least about 60% (e.g. about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, or about 99%) sequence identity with the amino acid encoded by SEQ ID NO: 1.

In embodiments, a hyaluronidase of the disclosure comprises an amino sequence having at least about 60% (e.g. about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, or about 99%) sequence identity with the amino acid encoded by SEQ ID NO: 1, from which nucleotides 1471 to 1527, corresponding to the carboxy-terminal domain, have been deleted.

In embodiments, a hyaluronidase of the disclosure comprises an amino sequence having at least about 60% (e.g. about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, or about 99%) sequence identity with the amino acid encoded by SEQ ID NO: 9.

In embodiments, a hyaluronidase of the disclosure comprises an amino sequence having at least about 60% (e.g. about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, or about 99%) sequence identity with SEQ ID NO: 8.

In embodiments, a hyaluronidase of the disclosure comprises an amino sequence having at least about 60% (e.g. about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, or about 99%) sequence identity with SEQ ID NO: 10.

In embodiments, an oncolytic adenovirus used in the present disclosure comprises one or more regulatory elements, modifications, or variations that provide for tumor-specific enzyme expression or viral replication.

In embodiments, expression of the hyaluronidase enzyme is controlled by a promoter operational in tumor cells. In embodiments, the expression of the hyaluronidase enzyme is controlled by a promoter operational in the retinoblastoma, Ewing sarcoma, or neuroblastoma cells to be treated. Optionally, in embodiments, the expression of the enzyme is controlled by a promoter operational in animal cells. Optionally, in embodiments, the promoter is selected from the cytomegalovirus promoter, the adenovirus major late promoter, the SV40 promoter, the herpes simplex virus thymidine kinase promoter, the RSV promoter, the EF1-a promoter, the beta-actin promoter, the human IL-2 promoter, the human IL-4 promoter, the IFN promoter, the E2F promoter, the human GM-CSF promoter, or combinations thereof.

In embodiments, the promoter that regulates the expression of the enzyme can be present naturally in the adenovirus, as is the case with the adenovirus major late promoter. In embodiments, the promoter can also be inserted together with the sequence encoding the enzyme. In embodiments, the promoter is the adenovirus major late promoter and this is already located in the oncolytic adenovirus genome. In embodiments, it is not necessary to introduce the promoter together with the hyaluronidase enzyme sequence, but rather the latter is introduced into the oncolytic adenovirus genome in such a way that it remains under the control of the promoter.

In embodiments, an oncolytic adenovirus used in the present disclosure comprises additional sequences that allow promotion or optimization of the protein translation of the sequence encoding the hyaluronidase enzyme. In embodiments, the sequences are inside or outside the hyaluronidase enzyme gene. For example, in embodiments, the additional sequences are selected from a sequence of cutting and splicing that allows RNA to be processed, IRES (internal ribosome entry site) sequences, the picornavirus sequence 2A, or combinations thereof.

### Replication Machinery Specific for Tumor Cells

In embodiments, tumor cell-specific replication machinery comprised by an oncolytic virus used in the present disclosure is machinery that causes the oncolytic adenovirus to replicate itself in a specific form in tumor cells and not in healthy, non-tumor, or normal cells. In embodiments, the machinery can take different forms if, as a result, it provides oncolytic adenoviruses with a replicative capacity solely, or largely, in the tumor cells, e.g., retinoblastoma, Ewing sarcoma, or neuroblastoma cells. In other words, in embodiments, adenoviruses with selective replication in which their replication machinery requires the virus to enter the cancer or tumor cells in order for the virus to replicate itself. In embodiments, the oncolytic adenovirus used can have modifications in its genome sequence that give it selective replication in tumor cells.

In embodiments, this is achieved by the incorporation of a specific tissue promoter or a specific tumor promoter, where the promoter controls the expression of one or more genes in the group E1a, E1b, E2 and E4. In embodiments, the promoter is selected from the E2F promoter, the telomerase hTERT promoter, the tyrosinase promoter, the prostate-specific antigen (PSA) promoter, the alpha-fetoprotein promoter, the COX-2 promoter, as well as artificial promoters formed by various transcription factor binding sites such as binding sites for hypoxia-inducible factor (HIF-1), the ETS transcription factor, the tumor cytotoxic factor (TCF), the E2F transcription factor or the Sp1 transcription factor. In embodiments, the promoter controls the expression of E1a.

In embodiments, the treatment is of tumors with an aberrant Rb-E2F pathway. In healthy, non-tumor, or normal cells, E1A protein expressed by the adenovirus, dissociates the host cell Rb-E2F complex and allows free E2F protein to drive both cell division and viral replication. In tumor cells with aberrant Rb-E2F machinery, free E2F allows the cells to divide (and virus to replicate) continuously without being initiated by virus E1A. Genetic modifications that suppress normal viral E1A expression thus prevent viral replication in healthy, non-tumor, or normal cells while permitting replication in tumor cells. Almost all solid tumors have aberrant Rb-E2F pathways, including, but not limited to retinoblastoma, Ewing sarcoma, and neuroblastoma. In embodiments, an oncolytic adenovirus used in the present disclosure is characterized by the deletion of the Rb binding domain. In embodiments, an oncolytic adenovirus used in the present disclosure is characterized by the deletion Δ24, which affects the interaction of E1a with the retinoblastoma protein, and the insertion of four sites binding to E2F-1 and one site binding to Sp1 into the endogenous E1a promoter to control the expression of E1a. Said DNA sequence corresponds to SEQ ID NO: 2.

Furthermore, it is envisaged that an oncolytic adenovirus used in the present disclosure, in embodiments, comprises one or more modifications in the capsid allowing improved biodistribution and reduced clearance of the oncolytic adenovirus from the body. In embodiments, an oncolytic adenovirus used in the present disclosure has the capsid modified to reduce its sequestration and destruction in the liver, such that the heparan sulfate binding domain KKTK present in the adenovirus fiber has been replaced, e.g. by the domain RGDK. The modification relates to positions 91 to 94 of the adenovirus fiber, taking the standard sequence of the adenovirus serotype 5 fiber as the reference. The sequence SEQ ID NO: 4 shows the complete sequence of the adenovirus type 5 fiber protein with the modified version in its heparan sulfate binding domain (modification RGDK).

Therefore, in embodiments, an oncolytic adenovirus used in the present disclosure is generated from a human adenovirus serotype 5 and comprises:
a sequence encoding a hyaluronidase enzyme inserted into its genome, optionally the human testicular hyaluronidase enzyme sequence (PH20, SEQ ID NO: 1) from which the sequence corresponding to the membrane-binding carboxy-terminal domain has been deleted in order for the enzyme to be soluble, optionally the sequence encodes a hyaluronidase enzyme having the amino acid sequence of SEQ ID NO: 10;
oncolytic adenoviral replication occurs in tumor cells with an aberrant Rb-E2F pathway and not in healthy, non-tumor, or normal cells; optionally the oncolytic adenovirus contains a deletion of the Rb binding domain or the deletion Δ24, which affects the interaction of E1a with the Rb protein, and the insertion of four binding sites to E2F-1 and one binding site to Sp1 into the endogenous E1a promoter to control the expression of E1a; and/or
modification of the adenovirus capsid to improve biodistribution and reduce clearance of the oncolytic adenovirus from the body; optionally the heparan sulfate binding domain KKTK present in the adenovirus fiber has been replaced, e.g. by the domain RGDK.

In embodiments, an oncolytic adenovirus of the present disclosure comprises a nucleotide sequence having at least about 60% (e.g. about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, or about 99%) sequence identity with the sequence of SEQ ID NO: 3. In embodiments, the oncolytic adenovirus has a nucleotide sequence of SEQ ID NO: 3.

In embodiments, an oncolytic adenovirus of the present disclosure comprises a nucleotide sequence having at least about 60% (e.g. about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, or about 99%) sequence identity with the sequence of VCN-01. In embodiments, the oncolytic adenovirus has a nucleotide sequence of VCN-01.

### Methods of Making Oncolytic Adenoviruses of the Disclosure

In embodiments, to construct an oncolytic adenovirus to be used in the present disclosure, use is made of any of the methods of construction of genetically modified adenovirus known in the field of gene therapy and virotherapy using adenoviruses. The most commonly used method is based on first constructing the desired genetic modification in a plasmid containing the adenoviral region to be modified, and then carrying out homologous recombination in bacteria with a plasmid containing the rest of the viral genome. As will be appreciated by those in the art, the protein sequences depicted herein can be encoded by any number of possible nucleic acid sequences, due to the degeneracy of the genetic code.

In embodiments, the nucleic acids encoding the components of the disclosure can be incorporated into oncolytic adenoviruses as is known in the art, and depending on the host cells, used to produce the hyaluronidase of the disclosure. Generally, the nucleic acids are operably linked to any number of regulatory elements (promoters, origin of replication, selectable markers, ribosomal binding sites, inducers, etc.).

In embodiments, an oncolytic adenovirus used in the present disclosure is propagated and amplified in cell lines normally used in the field of gene therapy and virotherapy such as the lines HEK-293 (Reference number: ATCC CRL-1573) and A549 (Reference number: ATCC CCL185). In embodiments, a method of propagation of the adenovirus is by infection of a cell line permitting the replication of the adenovirus. The lung adenocarcinoma line A549 is an example of a line with such characteristics. In embodiments, propagation takes place, for example, as follows: the A549 cells are grown on plastic cell culture plates and are infected using 100 virus particles per cell. Two days later, the cytopathic effect reflecting virus production can be observed as a clustering of the cells. In embodiments, the cells are collected and stored in tubes. In embodiments, after centrifuging at about 1000 g for about 5 minutes, the cell pellet is frozen and thawed three times, to lyse the cells. In embodiments, the resulting cell extract is centrifuged at about 1000 g for about 5 minutes and the supernatant, in which the viruses are located, is loaded over a cesium chloride gradient and centrifuged for about 1 hour at about 35,000 g. In embodiments, the virus band obtained from the gradient is loaded again over another cesium chloride gradient and centrifuged for about 16 hours at about 35,000 g. In embodiments, the virus band is collected and dialyzed opposite PBS-10% glycerol. In embodiments, the virus dialysate is aliquoted and stored at about-80° C. The number of plaque-forming units and particles may be quantified following standard protocols known in the prior art. Phosphate-buffered saline (PBS) with 5% glycerol is a standard formulation for storing adenoviruses and is used in embodiments. However, new formulations improving virus stability have been described and are used in embodiments.

### Topoisomerase I Inhibitors

Topoisomerase inhibitors can inhibit cell proliferation by, e.g., preventing DNA replication, stimulating DNA damage and inducing cell cycle arrest.

Topotecan is a semisynthetic derivative of the cytotoxic alkaloid, camptothecin. SN-38 is the active topoisomerase I-inhibiting metabolite the prodrug irinotecan (CPT-11). Generally, topoisomerase I inhibitors cause cell cycle arrest in the S-phase by stabilizing the complex between topoisomerase I and DNA, thereby inhibiting re-ligation of topoisomerase I-mediated single-strand DNA breaks and producing potentially lethal double-strand DNA breaks.

In embodiments, topoisomerase I inhibitors or prodrugs thereof, e.g., topotecan, SN-38, or irinotecan, are administered in various ways to treat retinoblastoma, Ewing sarcoma, or neuroblastoma, including systemic, intra-arterial, and intravitreal administration.

In embodiments, for systemic administration, the topoisomerase I inhibitor or prodrug thereof, e.g., topotecan, SN-38, or irinotecan, is injected intravenously or administered orally. The topoisomerase I inhibitor or prodrug thereof, e.g., topotecan, SN-38, or irinotecan, then enters the bloodstream and is distributed throughout the body. Irinotecan is converted to the active topoisomerase I inhibitor SN-38 by liver and tumor carboxylesterase enzymes. In embodiments, systemic chemotherapy is used to treat children with bilateral retinoblastoma, or to treat children whose unilateral retinoblastoma cannot be treated by focused intervention such as cryotherapy or transpupillary thermotherapy or for whom OAC is not available. In embodiments, systemic chemotherapy is also used for to treat primary and metastatic Ewing sarcoma or neuroblastoma. For OAC, the chemotherapy drug is injected directly into the ophthalmic artery, the main artery that supplies blood to the eye. In many cases, this administration method has allowed doctors to save an eye that otherwise would have needed to be removed. Much lower doses of chemotherapy are used in this approach. For intravitreal chemotherapy, the chemotherapy drug is injected directly into the vitreous humor. This is sometimes used (along with systemic or intra-arterial chemo) to treat tumors that are widespread within the eye and have not been helped by other treatments.

In embodiments, the topoisomerase I inhibitor or prodrug thereof, e.g., topotecan, SN-38, or irinotecan, is administered intravenously, intraarterially, intravitreally, intrathecally, intraventricularly, or orally.

In embodiments, the methods of the present disclosure improve and/or increase and/or enhance antitumor efficacy compared to treatment with the topoisomerase I inhibitor or a prodrug thereof without the oncolytic adenovirus. In embodiments, the method makes the patient suitable for treatment with a combination therapy of more than one cancer therapies.

In embodiments, without wishing to be bound by theory, administration of the oncolytic adenovirus reduces apoptotic action of topotecan as compared to monotherapy. In embodiments, without wishing to be bound by theory, administration of topotecan following administration of the oncolytic adenovirus leads to S-phase cell cycle arrest. In embodiments, without wishing to be bound by theory, the S-phase cell cycle arrest results in increased infectivity of the oncolytic adenovirus as compared to treatment with the oncolytic adenovirus without the topoisomerase I inhibitor or prodrug thereof. In embodiments, without wishing to be bound by theory, administration of topotecan following administration of the oncolytic adenovirus leads to increased E2F-1, p21 and/or cyclin E1 expression as compared to treatment with the oncolytic adenovirus without the topoisomerase I inhibitor or prodrug thereof. In embodiments, without wishing to be bound by theory, the increased E2F-1 expression results in increased oncolytic activity of the oncolytic adenovirus as compared to treatment with the oncolytic adenovirus without the topoisomerase I inhibitor or prodrug thereof. In embodiments, without wishing to be bound by theory, the increased infectivity and oncolytic activity of the oncolytic adenovirus occurs without substantially increasing replication of the oncolytic adenovirus.

In embodiments, without wishing to be bound by theory, sequential treatments in which the oncolytic adenovirus is administered first, followed by systemic topotecan, enhance or increase cell infection and/or anticancer efficacy of the oncolytic adenovirus, addressing one of the main limitations of oncolytic virus therapies, i.e. limited penetration and distribution into the tumor.

### Formulations

The present disclosure provides the described oncolytic adenovirus and/or topoisomerase I inhibitor or prodrug thereof, e.g., topotecan, SN-38, or irinotecan, composition in various formulations. Any oncolytic adenovirus and/or topoisomerase I inhibitor or prodrug thereof, e.g., topotecan, SN-38, or irinotecan, described herein can take the form of solutions, emulsions, suspensions, delayed-release formulations, sustained-release formulations, controlled-release formulations, or any other form suitable for parenteral use.

It will be understood that the compositions used in the present disclosure are used in a pharmaceutically acceptable presentation. This means that any oncolytic adenovirus and/or topoisomerase I inhibitor or prodrug thereof, e.g., topotecan, SN-38, or irinotecan, described herein can be administered to a subject as a component of a composition that comprises a pharmaceutically acceptable carrier or vehicle. Such compositions can optionally comprise a suitable amount of a pharmaceutically acceptable excipient so as to provide the form for proper administration.

Pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxiliary, stabilizing, thickening, lubricating, and coloring agents can be used. In embodiments, the pharmaceutically acceptable excipients are sterile when administered to a subject. Water is a useful excipient when any agent described herein is administered intravenously. Saline solutions and aqueous dextrose and glycerol solutions can also be employed as liquid excipients, specifically for injectable solutions. Suitable pharmaceutical excipients also include starch, glucose, cellulose, hypromellose, lactose, sucrose, trehalose, malt, rice, flour, chalk, silica gel, sodium stearate, glycerol monostearate, talc, sodium chloride, dried skim milk, glycerol, propylene, glycol, povidone, crosspovidone, water, ethanol and the like. Any agent described herein, if desired, can also comprise minor amounts of wetting or emulsifying agents, or pH buffering agents. Other examples of suitable pharmaceutical excipients are described in Remington's Pharmaceutical Sciences 1447-1676 (Alfonso R. Gennaro eds., 19th ed. 1995), incorporated herein by reference.

### Administration and Dosages

It will be appreciated that the actual dose of the oncolytic adenovirus and/or topoisomerase I inhibitor or prodrug thereof, e.g., topotecan, SN-38, or irinotecan, to be administered according to the present disclosure will vary according to different parameters, for example the volume of the vitreous cavity, the size and stage of the retinoblastoma, Ewing sarcoma, or neuroblastoma tumor to be treated, the age and weight of the patient to be treated, the particular dosage form, and the mode of administration. It will be understood that the doses of oncolytic adenovirus and co-administered of topoisomerase I inhibitor or prodrug thereof, e.g., topotecan, SN-38, or irinotecan, must be sufficient to ensure the combination of the method produces a positive therapeutic effect on the retinoblastoma, Ewing sarcoma, or neuroblastoma. In embodiments, the positive therapeutic effect for retinoblastoma, Ewing sarcoma, or neuroblastoma refers to halted tumor growth, or reductions in tumor volume, or prevention of tumor penetration into surrounding tissues, or prevention of metastases.

Many factors that may modify the action of the oncolytic adenovirus and/or topoisomerase I inhibitor or prodrug thereof, e.g., topotecan, SN-38, or irinotecan, (e.g., body weight, gender, diet, time of administration, route of administration, rate of excretion, condition of the subject, drug combinations, genetic disposition and reaction sensitivities) can be considered by those skilled in the art. Administration can be carried out continuously or in one or more discrete doses up to the maximum tolerated dose. Optimal administration rates for a given set of conditions can be ascertained by those skilled in the art using conventional evaluation of dosage administration and dose tolerability.

Compositions of the present disclosure, e.g., compositions comprising an oncolytic adenovirus disclosed herein, are administered by any route ensuring that the necessary amount of the oncolytic adenovirus reaches the tumor site. In the case of retinoblastoma, compositions of the present disclosure, e.g., compositions comprising an oncolytic adenovirus disclosed herein, are injected by any route ensuring that the necessary amount of the oncolytic adenovirus reaches the interior of the eyeball. In the case of Ewing sarcoma or neuroblastoma, compositions of the present disclosure, e.g., compositions comprising an oncolytic adenovirus disclosed herein, are injected by any route ensuring that the necessary amount of the oncolytic adenovirus reaches the tumor site. Optional routes of administration include, but are not limited to, intravenous injection, intraarterial injection, intratumoral injection, intraocular injection, intraperitoneal injection, intrathecal injection, and intravitreal injection. Intraocular or intravitreal injections in retinoblastoma patients have the advantage that they are performed into an organ, the eye, which provides an immunoprivileged environment, facilitating a lower immune response, or no response, to the oncolytic adenovirus comprised in the composition. This makes it possible to ensure, to a large extent, that the action of the oncolytic adenovirus will remain confined to the eye, since if the oncolytic adenovirus should ever leave the eye, it could be neutralized by the immune system. Therefore, in embodiments, the preferred compositions comprising an oncolytic adenovirus disclosed herein take an appropriate form for intravitreal, intraarterial, intrathecal, or intraocular administration or injection. Another possible route of administration for the compositions provided herein is intratumoral injection.

In embodiments, the oncolytic adenovirus and/or topoisomerase I inhibitor or prodrug thereof, e.g., topotecan, SN-38, or irinotecan, may be administered, for example, more than once daily (e.g., about two, about three, about four, about five, about six, about seven, about eight, about nine, or about ten times per day), about once per day, about every other day, about every third day, about once a week, about once every two weeks, about once every month, about once every two months, about once every three months, about once every six months, or about once every year.

In embodiments, the subject is administered one intrathecal injection of VCN-01 concomitant with systemic or intrathecal cycles of topotecan. In embodiments, the subject has neuroblastoma or retinoblastoma, optionally leptomeningeal metastatic retinoblastoma or CNS-metastatic retinoblastoma. In embodiments, the subject is administered VCN-01 and topotecan or irinotecan systemically. In embodiments, the subject has an extracranial disseminated disease, optionally Ewing sarcoma. In embodiments, the subject has an aggressive and/or chemorefractory pediatric solid tumor expressing E2F-1. In embodiments, the VCN-01 and/or topotecan is co-administered with one or more local and/or systemic corticosteroids. In embodiments, the one or more local and/or systemic corticosteroids are selected from hydrocortisone, prednisone, prednisolone, methylprednisolone, dexamethasone, betamethasone, alsosterone, budesonide, fluticasone, flunisolide, ciclesonide, mometasone, beclomethasone, triamcinolone, and tixocortol. In embodiments, the one or more local and/or systemic corticosteroids are selected from methylprednisolone, dexamethasone, betamethasone, and triamcinolone.

### Definitions

As used herein, “a,” “an,” or “the” can mean one or more than one.

Further, the term “about” when used in connection with a referenced numeric indication means the referenced numeric indication plus or minus up to 10% of that referenced numeric indication. For example, the language “about 50%” covers the range of 45% to 55%.

An “effective amount,” when used in connection with medical uses is an amount that is effective for providing a measurable treatment, prevention, or reduction in the rate of pathogenesis of a disorder of interest.

As used herein, something is “decreased” if a read-out of activity and/or effect is reduced by a significant amount, such as by at least about 10%, at least about 20%, at least about 30%, at least about 40%, at least about 50%, at least about 60%, at least about 70%, at least about 80%, at least about 90%, at least about 95%, at least about 97%, at least about 98%, or more, up to and including at least about 100%, in the presence of an agent or stimulus relative to the absence of such modulation. As will be understood by one of ordinary skill in the art, in embodiments, activity is decreased and some downstream read-outs will decrease but others can increase.

Conversely, activity is “increased” if a read-out of activity and/or effect is increased by a significant amount, for example by at least about 10%, at least about 20%, at least about 30%, at least about 40%, at least about 50%, at least about 60%, at least about 70%, at least about 80%, at least about 90%, at least about 95%, at least about 97%, at least about 98%, or more, up to and including at least about 100% or more, at least about 2-fold, at least about 3-fold, at least about 4-fold, at least about 5-fold, at least about 6-fold, at least about 7-fold, at least about 8-fold, at least about 9-fold, at least about 10-fold, at least about 50-fold, at least about 100-fold, in the presence of an agent or stimulus, relative to the absence of such agent or stimulus.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the compositions and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of terms such as including, containing, or having, is used herein to describe and claim the disclosure, the present disclosure, or embodiments thereof, may alternatively be described using alternative terms such as “consisting of” or “consisting essentially of.”

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

In embodiments, the terms “patient” and “subject” are used interchangeably. In embodiments, the subject and/or animal is a mammal, e.g., a human, mouse, rat, guinea pig, dog, cat, horse, cow, pig, rabbit, sheep, or non-human primate, such as a monkey, chimpanzee, or baboon. In embodiments, the subject and/or animal is a non-mammal, such, for example, a zebrafish.

In embodiments, methods of the invention are useful in treatment a human subject. In embodiments, the human is a pediatric human. In embodiments, the human is an adult human. In embodiments, the human is a geriatric human. In embodiments, the human may be referred to as a patient. In embodiments, the human is a female. In embodiments, the human is a male.

In embodiments, the human has an age in a range of from about 1 to about 18 months old, from about 18 to about 36 months old, from about 1 to about 5 years old, from about 5 to about 10 years old, from about 10 to about 15 years old, from about 15 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old, from about 80 to about 85 years old, from about 85 to about 90 years old, from about 90 to about 95 years old or from about 95 to about 100 years old.

The amount of each component in the compositions described herein needed for achieving a therapeutic effect may be determined empirically in accordance with conventional procedures for the particular purpose. Generally, for administering therapeutic agents (e.g., oncolytic adenoviruses and/or topoisomerase I inhibitor or prodrug thereof, e.g., topotecan, SN-38, or irinotecan, compositions described herein) for therapeutic purposes, the therapeutic agents are given at a pharmacologically effective dose. A “pharmacologically effective amount,” “pharmacologically effective dose,” “therapeutically effective amount,” or “effective amount” refers to an amount sufficient to produce the desired physiological effect or amount capable of achieving the desired result, particularly for treating the disorder or disease. An effective amount as used herein would include an amount sufficient to, for example, delay the development of a symptom of the disorder or disease, alter the course of a symptom of the disorder or disease (e.g., slow the progression of a symptom of the disease), reduce or eliminate one or more symptoms or manifestations of the disorder or disease, and reverse a symptom of a disorder or disease. Therapeutic benefit also includes halting or slowing the progression of the underlying disease or disorder, regardless of whether improvement is realized.

Effective amounts, toxicity, and therapeutic efficacy can be determined by standard pharmaceutical procedures in cell cultures, tissue samples, tissue homogenates or experimental animals, e.g., for determining the LD50 (the dose lethal to about 50% of the population) and the ED50 (the dose therapeutically effective in about 50% of the population) or the maximum tolerated dose. The dosage can vary depending upon the dosage form employed and the route of administration utilized. The dose ratio between toxic and therapeutic effects is the therapeutic index and can be expressed as the ratio LD50/ED50. In embodiments, compositions and methods that exhibit large therapeutic indices are preferred. A therapeutically effective dose can be estimated initially from in vitro assays, including, for example, cell culture assays or measurements or methane production in stool samples. Also, a dose can be formulated in animal models to achieve a circulating plasma concentration range that includes the IC50 as determined in cell culture, or in an appropriate animal model. Levels of the described compositions in plasma can be measured, for example, by high performance liquid chromatography. The effects of any particular dosage can be monitored by a suitable bioassay. The dosage can be determined by a physician and adjusted, as necessary, to suit observed effects of the treatment.

In embodiments, the effect will result in a quantifiable change of at least about 10%, at least about 20%, at least about 30%, at least about 50%, at least about 70%, or at least about 90%. In embodiments, the effect will result in a quantifiable change of about 10%, about 20%, about 30%, about 50%, about 70%, or even about 90% or more. Therapeutic benefit also includes halting or slowing the progression of the underlying disease or disorder, regardless of whether improvement is realized.

As used herein, “methods of treatment” are equally applicable to use of a composition for treating the diseases or disorders described herein and/or compositions for use and/or uses in the manufacture of a medicaments for treating the diseases or disorders described herein.

### EXAMPLES

### Example 1: Treatment of Retinoblastoma with the Oncolytic Adenovirus VCN-01 and Topotecan

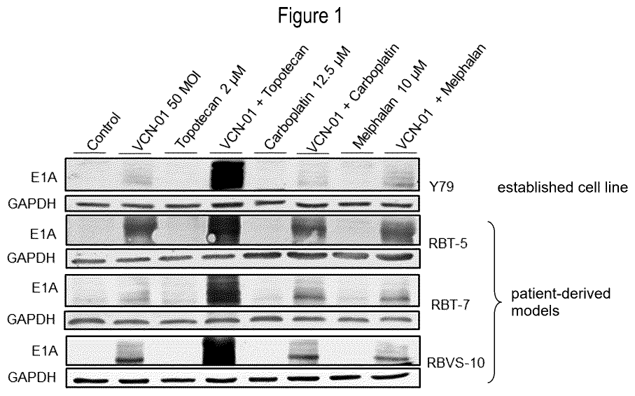

2 million HSJD-RBT-5, HSJD-RBT-7, HSJD-RBVS-10 and Y79 retinoblastoma cells were infected with 50 MOIs of VCN-01, which has a nucleotide sequence of SEQ ID NO: 3. After 24 h, the cells were treated with topotecan (2 μM), carboplatin (12.5 μM) or melphalan (10 μM). Total cell lysates were extracted after 48 h, and immunoblotting of E1a was carried out, with GAPDH as loading control. E1A is the first protein expressed by adenovirus and drives the expression of remaining viral proteins. The results are shown in FIG. 1 and demonstrate that topotecan, but not carboplatin or melphalan, enhanced expression of VCN-01 proteins in retinoblastoma cells, including 1 cell line and 3 patient-derived models.

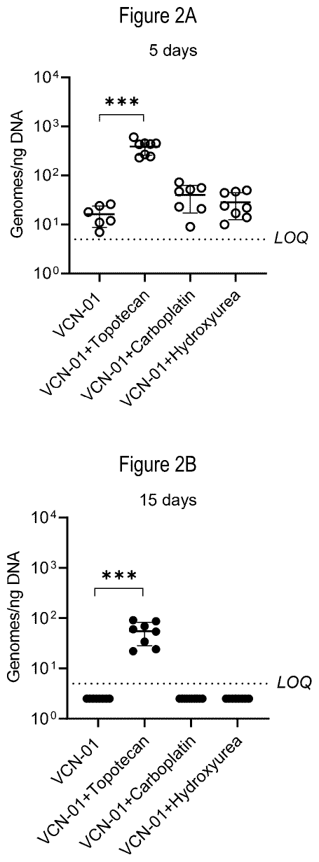

Y79 cells were inoculated in both flanks of athymic nude mice. Upon engraftment (tumor volumes ranging 100-200 mm³), VCN-01 was injected intratumorally (3×10E⁹ vp/tumor; single dose, at day 1). Mice then received either topotecan (0.6 mg/kg/day) or hydroxyurea (200 mg/kg/day) daily during days 1-5, or carboplatin (40 mg/kg), single dose at day 1. Mice were sacrificed at two different times: 5 days post VCN-01 inoculation or 15 days post VCN-01 inoculation, and the viral genomes were quantitated. The results, shown in FIGS. 2A-B, demonstrate that administration of systemic topotecan after intratumoral VCN-01, but not carboplatin, increases VCN-01 genome content in retinoblastoma tumor models in vivo. It is noted that hydroxyurea is a model drug to arrest retinoblastoma cells in S phase. Although it could be hypothesized that the enhancing activity of topotecan on VCN-01 is due to arrest of retinoblastoma cells in S phase (shared with hydroxyurea), the enhancement of VCN-01 replication in vivo appears to be specific to topotecan.

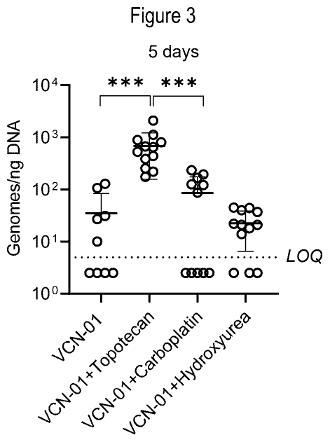

HSJD-RBT-7 cells were inoculated in both flanks of athymic nude mice. Upon engraftment (tumor volumes ranging 100-200 mm³), VCN-01 was injected intratumorally (3×10E⁹ vp/tumor; single dose, at day 1). Mice then received either topotecan (0.6 mg/kg/day) or hydroxyurea (200 mg/kg/day) daily during days 1-5, or carboplatin (40 mg/kg), single dose at day 1. Mice were sacrificed at 5 days post VCN-01 inoculation, and the viral genomes were quantitated. The results, shown in FIG. 3, demonstrate that administration of systemic topotecan after intratumoral VCN-01, but not carboplatin, increases VCN-01 genome content in retinoblastoma tumor models in vivo.

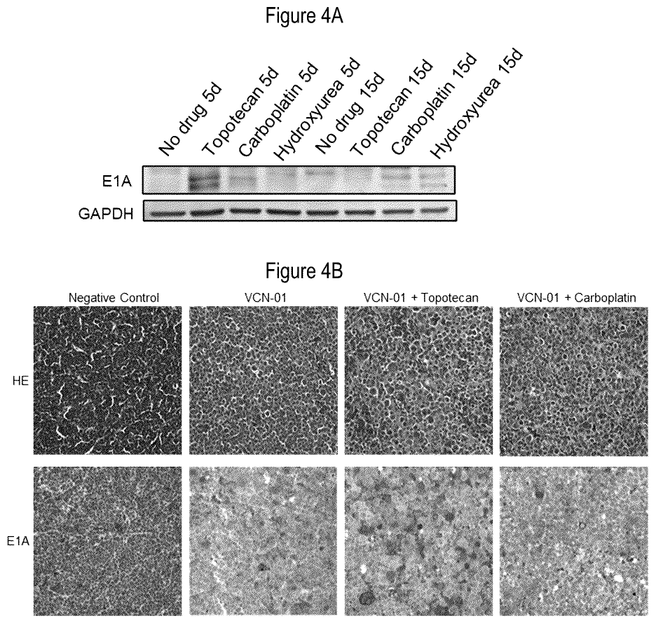

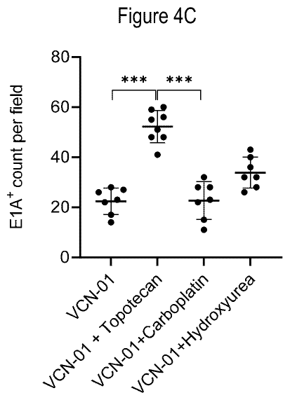

Y79 cells were inoculated in both flanks of athymic nude mice. Upon engraftment (tumor volumes ranging 100-200 mm³), VCN-01 was injected intratumorally (3×10E⁹ vp/tumor; single dose, at day 1). Mice then received either topotecan (0.6 mg/kg/day) or hydroxyurea (200 mg/kg/day) daily during days 1-5, or carboplatin (40 mg/kg), single dose at day 1. Mice were sacrificed at two different times: 5 days post VCN-01 inoculation or 15 days post VCN-01 inoculation. The results, shown in FIGS. 4A-C, demonstrate that administration of systemic topotecan after intratumoral VCN-01, but not carboplatin, enhances VCN-01 activity in retinoblastoma tumor models in vivo. The immunoblotting shows E1A and loading control GAPDH of subcutaneous tumors extracted at 5 or 15 days (FIG. 4A). The immunostaining shows representative images of E1A staining in subcutaneous tumors obtained 5 days after VCN-01 injection and topotecan or carboplatin treatment (FIG. 4B). The graph in FIG. 4C is of E1A-positive cell counts in 7 different fields of each sample, with each dot representing one field.

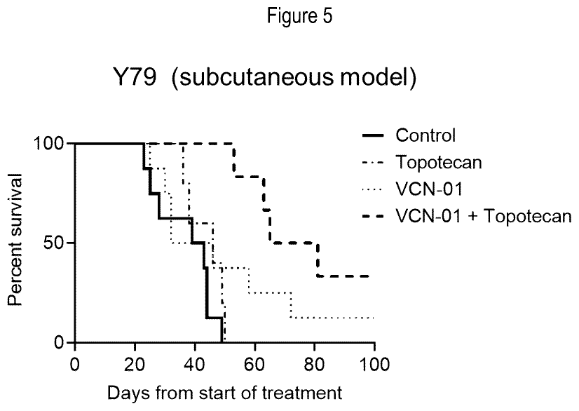

The effect of topotecan on the antitumor activity of VCN-01 on Y79 subcutaneous model is shown in FIG. 5. The dosages were as follows—VCN-01: intratumoral, 3×10⁹ vp/tumor, at day 1 (single dose); and topotecan: systemic, 0.6 mg/kg/day, at days 1, 2, 3, 4 and 5.

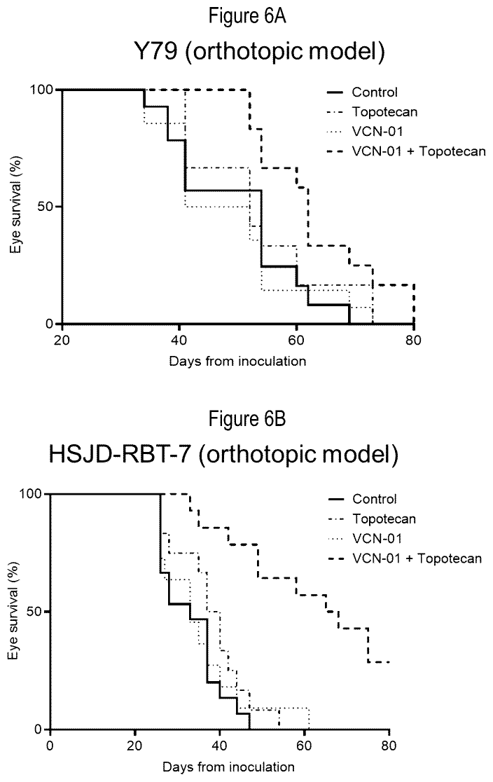

The effect of topotecan on the antitumor activity of VCN-01 on Y79 and HSJD-RBT-7 orthotopic (intraocular retinoblastoma) models is shown in FIGS. 6A-B. The dosages were as follows-VCN-01: intratumoral, 3×10⁹ vp/tumor, at day 1 (single dose); and topotecan: systemic, 0.6 mg/kg/day, at days 1, 2, 3, 4 and 5. Orthotopic xenografts models reproduce intraocular tumor growth and recapitulate human disease and molecular markers of dissemination.

### Example 2: In Vitro Evaluation of VCN-01 in Combination with Topoisomerase I Inhibitors

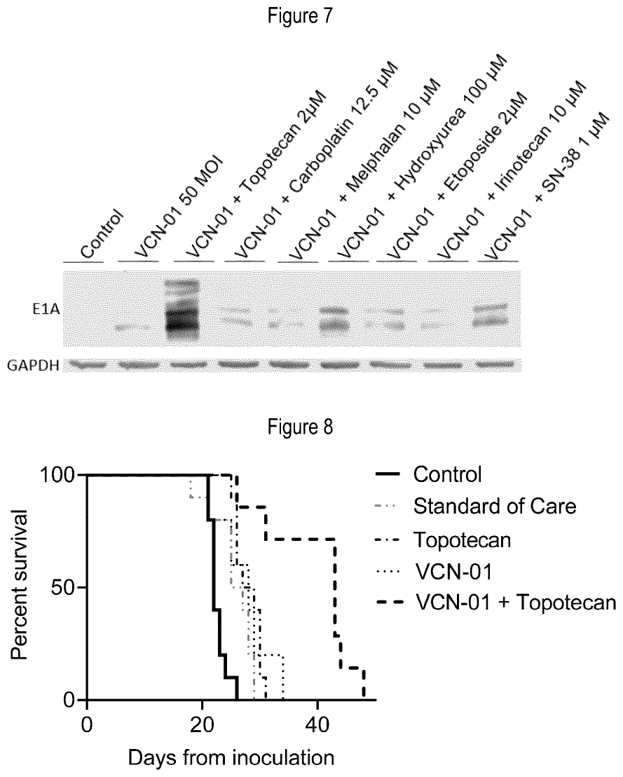

2 million Y79 retinoblastoma cells were infected with 50 MOIs of VCN-01. After 24 h, the cells were treated with topotecan (2 M), carboplatin (12.5 μM) melphalan (10 μM), hydroxyurea (100 μM), etoposide (2 μM), irinotecan (10 μM), or SN-38 (1 μM). Total cell lysates were extracted after 48 h, and immunoblotting of E1a was carried out, with GAPDH as loading control. The results are shown in FIG. 7 and demonstrate that SN-38, another topoisomerase 1 inhibitor of the camptothecin family increases expression of VCN-01 proteins in retinoblastoma cells (Y79 model) similarly to topotecan. The prodrug of SN-38, irinotecan, does not work in vitro, due to lack of conversion to SN-38 in the absence of carboxylesterases. Interestingly, another topoisomerase inhibitor, etoposide, does not induce the same effect. Etoposide is topoisomerase 2 inhibitor.

### Example 3: Treatment of Intracerebral Retinoblastoma With Systemic Topotecan and Intrathecal Administration of VCN-01

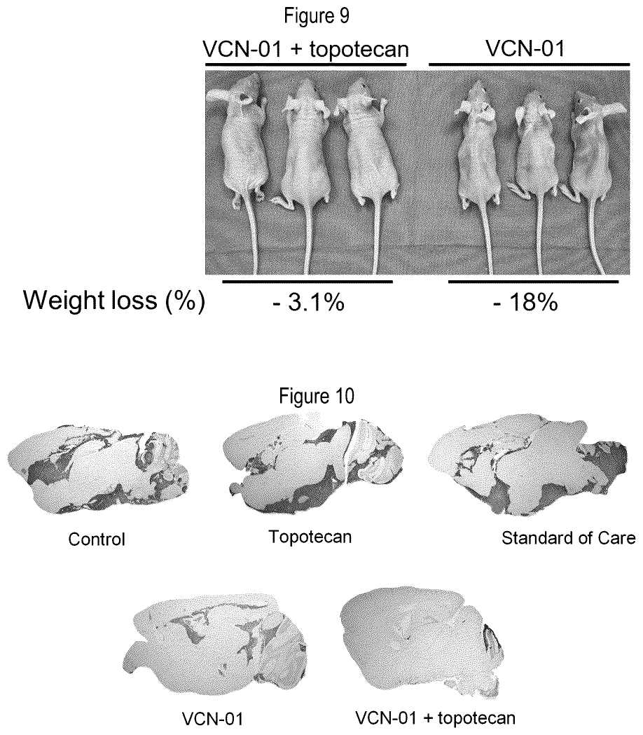

1 million retinoblastoma HSJD-RBT-7 cells in 25 UL Matrigel were inoculated intrathecally, specifically in the fourth ventricle of the brain, of athymic nude mice on Day 0. On day 6 after tumor cell inoculation, combination treatment was started, in which a single dose of 3×10⁹ vp/tumor VCN-01 in 25 UL was administered by stereotactic injection intrathecally (in the fourth ventricle of the brain), on the morning of day 6, and topotecan was administered systemically (intraperitoneal) at a concentration of 0.6 mg/kg/day, on the afternoons of days 6, 7, 8, 9, and 10. Such combination was compared to either of the treatments alone, and to standard of care treatment, which included carboplatin 40 mg/kg, systemic (intraperitoneal) administration at day 6 in combination with etoposide 6 mg/kg, intraperitoneal administration at days 7 and 8. The results are shown in FIG. 8 and demonstrate that combination treatment was more effective than either single treatment and to standard of care treatment. Additional results are shown in FIG. 9 and demonstrate that, 26 days after starting treatment, the VCN-01+topotecan mice experienced less weight loss than the mice treated only with VCN-01. By that time, all control mice had perished of disease. Additional results are shown in FIG. 10 and demonstrate that, 26 days after starting treatment, the VCN-01+topotecan mice had less tumor load (measured as amount of human cells stained with and anti-human nuclei antibody; areas stained in dark color in the brains) in the ventricular-intrathecal space than mice treated with each of the other treatments.

### Example 4: Treatment of Ewing Sarcoma with Topotecan and VCN-01

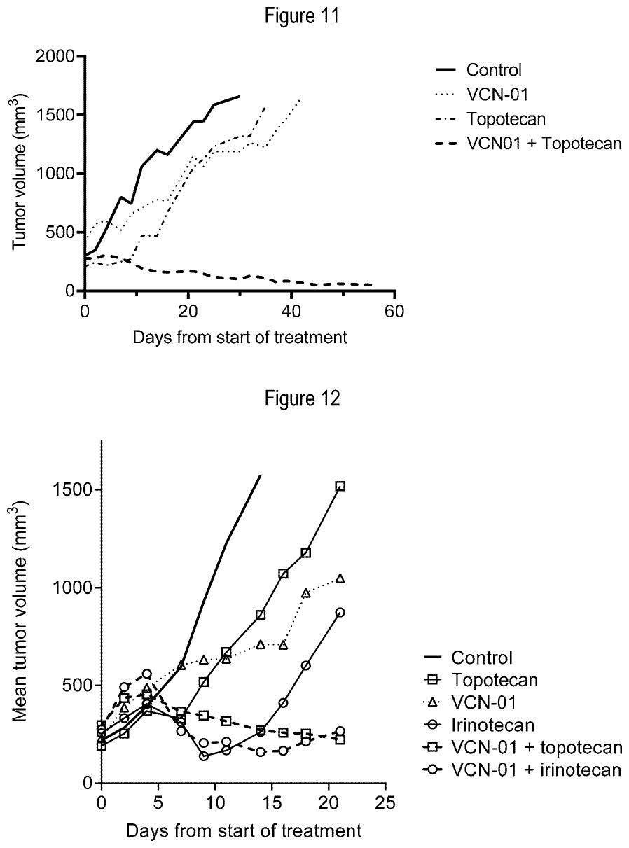

Patient-derived xenograft (PDX) of Ewing sarcoma (pediatric solid tumor) were implanted subcutaneously into athymic nude mice. Tumor model was HSJD-ES-033. Upon engraftment (tumor volumes ranging 100-500 mm³), combination treatment in which topotecan was administered systemically (intraperitoneal) at a concentration of 0.6 mg/kg/day, on Days 1, 2, 3, 4 and 5, and a single dose of 3×10⁹ vp/tumor VCN-01 was administered intratumorally on Day 1, was compared to either of the treatments alone. The results are shown in FIG. 11, which graphs mean tumor size for 2-4 tumors from each group, and demonstrate that combination treatment was more effective than either single treatment.

### Example 5: Treatment of Neuroblastoma with Topotecan or Irinotecan in Combination with VCN-01

PDX of neuroblastoma (pediatric solid tumor) were implanted subcutaneously into athymic nude mice. Tumor model was HSJD-NB-005. Upon engraftment (tumor volumes ranging 100-500 mm³), treatments were started. The activity of topotecan vs. irinotecan in combination with VCN-01 was evaluated. The following groups of mice were included in the study, with an n of 6 tumors per group:
Group 1: no treatment
Group 2: topotecan i.p. at 0.6 mg/kg, Day 1-5
Group 3: irinotecan i.p. at 10 mg/kg, Day 1-5
Group 4: VCN-01 i.t at 3E9 vp/tumor, Day 1
Group 5: VCN-01 i.t at 3E9 vp/tumor, Day 1 (morning)+topotecan i.p. at 0.6 mg/kg, Day 1-5
Group 6: VCN-01 i.t at 3E9 vp/tumor, Day 1 (morning)+irinotecan i.p. at 10 mg/kg, Day 1-5

The results are shown in FIG. 12 and demonstrate that combination treatments of topotecan and VCN-01 was more effective than either single treatment. Combination treatment of irinotecan and VCN-01 was clearly more effective than VCN-01, and slightly more effective than irinotecan.

### Example 6: Treatment of Pediatric Cancers with Topotecan in Combination with VCN-01

This example describes, inter alia, a study of the interaction of VCN-01 and chemotherapeutic agents topotecan, carboplatin and melphalan, all used in the standard of care of retinoblastoma and other pediatric cancers. Topotecan is a topoisomerase 1 inhibitor of the camptothecin family. These drugs produce double-strand breaks in the DNA (Pommier, “Topoisomerase I inhibitors: camptothecins and beyond,” Nature Reviews Cancer 2006; 6(10):789-802) and S-phase cell cycle arrest (Ohneseit et al., “Cell cycle effects of topotecan alone and in combination with irradiation,” Radiother Oncol 2005; 75(2):237-45). Carboplatin and melphalan are alkylating agents. This study demonstrated that topotecan enhances the infectivity of VCN-01 in cancer cells, providing a significant therapeutic benefit in several pediatric cancer xenografts expressing E2F-1, including intraocular retinoblastomas, central nervous system (CNS)-disseminated retinoblastoma and subcutaneous (s.c.) patient-derived xenografts (PDX) of Ewing sarcoma and neuroblastoma.

Cell cultures, tumor models, viral constructs and chemotherapy agents. Primary retinoblastoma cell cultures established from enucleated eyes of four human patients (Pascual-Pasto et al., “Preclinical platform of retinoblastoma xenografts recapitulating human disease and molecular markers of dissemination,” Cancer letters 2016; 380(1):10-19) were used. The Y79 cell line was from the ATCC (Manassas, VA, USA). Clinical details of the cell lines are in Table 1.

TABLE 1
Clinical details of the retinoblastoma cell models.

| | | | Age at | Age at | | |
|---|---|---|---|---|---|---|
| | Genomic | Tissue | diagnosis | enucleation | | |
| Cell model ID | alterations¹ | of origin | (months) | (months) | Laterality | Chemotherapy |
| HSJD-RBT-2 | RB1^{mut} | Tumor | 8 | 13 | Bilateral | Yes² |
| HSJD-RBT-5 | RB1^{mut} | Tumor | 7 | 24 | Unilateral | Yes³ |
| HSJD-RBT-7 | RB1^{mut} | Tumor | 6 | 6 | Unilateral | No |
| | MYCN^{A} | | | | | |
| HSJD-RBVS-10 | RB1^{mut} | Vitreous | 5 | 28 | Bilateral | Yes⁴ |
| | | humor | | | | |
| Y79 | RB1^{mut} | Tumor | 30 | 30 | Unilateral | No |
| | MYCN^{A} | | | | | |

¹RB1^{mut}: mutation in the gene RB1; MYCN^{A}: amplification of the gene MYCN.
²Five tandem doses of topotecan and melphalan in the ophthalmic artery.
³Six cycles of systemic carboplatin, etoposide and vincristine, three tandem doses of topotecan and melphalan in the ophthalmic artery and ruthenium brachytherapy.
⁴Eight cycles of systemic carboplatin, etoposide and vincristine, ten cycles of systemic cyclophosphamide and etoposide, one dose of intravitreous melphalan and three tandem doses of topotecan and melphalan in the ophthalmic artery.

Primary tumor cells were cultured in neural stem cell medium (serum-free medium with added growth factors) and Y79 cells were cultured in Roswell Park Memorial Institute (RPMI) medium supplemented with 10% FBS, as described (Pascual-Pasto et al., 2016). Short tandem repeat (STR) profiles of the primary cells are reported elsewhere (Cuadrado-Vilanova et al., “Identification of immunosuppressive factors in retinoblastoma cell secretomes and aqueous humor from patients,” The Journal of pathology 2022; 257(3):327-39). Ewing sarcoma and neuroblastoma PDX were from the HSJD repository and have clinically annotated data available (Pascual-Pasto et al., “Low Bcl-2 is a robust biomarker of sensitivity to nab-paclitaxel in Ewing sarcoma,” Biochem Pharmacol 2023; 208:115408; Aschero et al., “Prognostic value of xenograft engraftment in patients with metastatic high-risk neuroblastoma,” Pediatric blood & cancer 2023:e30318). Cell cultures and PDX are identified with the institutional prefix HSJD (Table 1), which is omitted in the text and figures for clarity purposes.

Cell proliferation. The cancer cells were incubated with VCN-01 and the chemotherapeutic agents. First, the antiproliferative activity of topotecan (ranging 10-0.0000256 μM), carboplatin (200-0.78 μM) or melphalan (10-0.00015 μM) was evaluated in retinoblastoma cells (2×10⁴ cells per well, in 96-well plates) infected with VCN-01 for the three previous days, at a concentration of 10 multiplicity of infection (MOI; i.e., transducing units of virus per cell). This concentration of virus was sub-toxic for incubations of six days in these cells (Pascual-Pasto et al., “Therapeutic targeting of the RB1 pathway in retinoblastoma with the oncolytic adenovirus VCN-01,” Science Translational Medicine 2019; 11 (476): eaat9321). Three days after the addition of drugs, cell viability was measured with the compound [3-(4,5-dimethylthiazol-2-yl)-5-(3-carboxymethoxyphenyl)-2-(4-sulfophenyl)-2H] tetrazolium (MTS assay; Promega, Fitchburg, WI, USA).

To address the sequence of the treatments on cell viability and apoptosis markers, topotecan (2 μM) and VCN-01 at 50 MOI (concentration sufficient to achieve viral protein transduction at early time points) was incubated with the sequence “topotecan first” (three days before VCN-01) or “VCN-01 first” (three days before topotecan). The experiments were controlled by adding culture medium instead of the second treatment.

Similar experiments were performed to address the antiproliferative activity of VCN-01 (100-0.4 MOI) in primary cultures of Ewing sarcoma and neuroblastoma PDX.

Genomic analyses. The expression of viral genes E1A, E1B and SPAM1 (recombinant PH20 hyaluronidase) and cell cycle genes such as CDKN1A and CDK6 was evaluated in retinoblastoma cells treated with the chemotherapeutic agents, VCN-01 and the combinations at different sequences.

The expression of viral genes E1A and E1B was studied in 6-well culture plates containing 2×10⁶ retinoblastoma cells. The cells were exposed to topotecan (2 μM) and VCN-01 (50 MOI) and incubated in the sequence “topotecan first” (24 h before VCN-01) or “VCN-01 first” (24 h before topotecan). The experiments were controlled by adding culture medium instead of topotecan. After 24 h of the second treatment, the cell pellets were collected and the mRNA was isolated with the TRIzol method (Thermo Fisher Scientific, Waltham, MA, USA). Real time quantitative polymerase chain reactions (RT-qPCR) were carried out to analyze gene expression using SYBR technology (Thermo Fisher Scientific). The primers were viral E1A (Forward 5′-ATC GAA GAG GTA CTG GCT GA-3′ (SEQ ID NO: 11), Reverse 5′-CCT CCG GTG ATA ATG ACA AG-3′ (SEQ ID NO: 12)) and E1B (Forward 5′-GAG GGT AAC TCC AGG GTG CG-3′ (SEQ ID NO: 13), Reverse 5′-TTT CAC TAG CAT GAA GCA ACC ACA-3′ (SEQ ID NO: 14)).

To assess the expression of genes involved in the cell cycle, 6-well culture plates containing 2×10⁶ retinoblastoma cells were used. VCN-01 was added at 50 MOI for 48 h, followed by the addition of topotecan (2 μM), carboplatin (12.5 μM) or culture medium. The cell pellets were collected 24 h later. The samples were plated in the TaqMan® Array of Human Cyclins & Cell Cycle Regulation (Thermo Fisher Scientific). This array contains 92 assays of cell cycle-associated genes and assays of GAPDH, 18S, HPRT1 and GUSB as endogenous control genes. the threshold cycle (Ct) value ≥35 was established as absence of expression. The mean expression value of the controls was used to normalize gene expression values. The thresholds for upregulation and downregulation were 2^{-ΔΔCt}≥2.0 and 2^{-ΔΔct}≤0.5, respectively (Livak and Schmittgen, “Analysis of relative gene expression data using real-time quantitative PCR and the 2 (−Delta Delta C(T)) Method,” Methods 2001; 25(4):402-8).

RT-qPCR was performed to validate results of the top genes overexpressed or downregulated in the array. SYBR primers were CDKN1A (Forward 5′-GGA CAG GAG AGG AAG ACC ATG T-3′ (SEQ ID NO: 15), Reverse 5′-TGG AGT GGT AGA ATT CTG TCA TGC-3′ (SEQ ID NO: 16) and CDK6 (Forward 5′-CCA GGC AGG CTT TTC ATT CA-3′ (SEQ ID NO: 17), Reverse 5′-AGG TCC TGG AAG TAT GGG TG-3′ (SEQ ID NO: 18).

To quantify the recombinant PH20 hyaluronidase gene (SPAM1) in cultures and mouse tissues, the SYBR primer: Forward 5′-TAC ACA CTC CTT GCT CCT GG-3′ (SEQ ID NO: 19), Reverse: 5′-CTT AGT CTC ACA GAG GCC AC-3′ (SEQ ID NO: 20) (Bazan-Peregrino et al., “VCN-01 disrupts pancreatic cancer stroma and exerts antitumor effects,” Journal for ImmunoTherapy of Cancer 2021; 9(11):e003254) was used.

To quantify the load of retinoblastoma cells in mouse tissues (i.e., to assess tumor burden), SYBR primers were used for the gene CRX (Forward: 5′-AGG TGG CTC TGA AGA TCA ATC TG-3′ (SEQ ID NO: 21), Reverse: 5′-TTA GCC CTC CGG TTC TTG AA-3′ (SEQ ID NO: 22) (Pascual-Pasto, 2016).

To assess the expression of E2F1, CXADR and ITGA5 in pediatric cancers, available data sets of fetal retina (GSE12621; n=12), pediatric brain (GSE44971 and GSE13564; n=28), muscle tissue (GSE17679; n=18), retinoblastoma (GSE29683; n=55), neuroblastoma (GSE16237; n=51) and Ewing sarcoma (GSE34620; n=44) were used. Data from CEL files was normalized and processed by the robust multichip averaged (RMA) algorithm using R statistical software and the Affymetrix library available through Bioconductor (Gautier et al., “affy—analysis of Affymetrix GeneChip data at the probe level,” Bioinformatics 2004; 20 (3): 307-15).

To quantify genome copies of VCN-01 in cell cultures or mouse tissues, total DNA was extracted and real time quantitative polymerase chain reactions (RT-qPCR) was performed as described (Cascallo et al., “Systemic toxicity-efficacy profile of ICOVIR-5, a potent and selective oncolytic adenovirus based on the pRB pathway,” Mol Ther 2007; 15(9):1607-15).

Protein expression. Proteins related to adenoviral infection, cell cycle and apoptosis of retinoblastoma cells exposed to chemotherapy agents and VCN-01 were studied.

In all experiments, 6-well culture plates containing 2×10⁶ cells were used. First, cells were infected with VCN-01 (50 MOI, 24 h), exposed to topotecan (2 μM), carboplatin (12.5 μM) or melphalan (12 μM), and the pellets were collected 24 h later for the analysis of apoptosis, cell cycle and infection markers. The experiments were controlled by adding culture medium instead of the second treatment. To address whether the sequence of treatments affected their interaction, sequence “topotecan first” (24 h before VCN-01) or “VCN-01 first” (24 h before topotecan) was applied. The analyses were expanded to other chemotherapeutic agents including hydroxyurea (100 M), etoposide (2 μM), irinotecan (10 μM) or SN-38 (1 μM).

In a time course experiment, cell cycle proteins were analyzed upon cell exposure to chemotherapeutic agents. Retinoblastoma cells were treated with topotecan (2 μM), carboplatin (12.5 μM), melphalan (12 μM), hydroxyurea (100 μM) or SN-38 (1 μM), and cell pellets were collected at 1, 2, 4, 8, 16, 24 and 48 h.

Cells were lysed with RIPA buffer (50 mM HEPES, 150 mM NaCl, 1 mM EDTA, 10% glycerol, 1% Triton-X 100, 0.1% SDS, 1% Na deoxycholate) to obtain whole protein extracts. Proteins were quantified with the BCA assay (Thermo Fisher Scientific) and the immunoblotting assays were performed as previously described (Mandigo et al., “Relevance of pRB Loss in Human Malignancies,” Clin Cancer Res 2022; 28(2):255-64).

For immunoblotting assays, primary antibodies were p53 (2527S, 1:1000, Cell Signaling, Danvers, MA, USA), E1A (adenovirus type 5 infection marker; ab33183, 1 μg/mL, Abcam, Cambridge, MA, USA), cleaved-poly(ADP-ribose) polymerase (cPARP; apoptosis marker; 9541S, 1:1000, Cell Signaling), p21 (2947, 1:1000, Cell Signaling), E2F-1 (VCN-01 promoter; 3742, 1:1000, Cell Signaling), Cyclin E1 (sc-377100, 1:200, Santa Cruz Biotechnology, Santa Cruz, CA, US), topoisomerase 1 (ab85038, 1:1000, Abcam), adenovirus type 5 (Hexon; ab6982, 1:1000, Abcam), β-tubulin (MAB374, 1:10,000, Millipore, Darmstadt, Germany) and GAPDH (1:10,000, Millipore). Near-infrared labeled secondary antibodies (Odyssey CLx, LI-COR Inc. Bad Homburg, Germany) were used for detection.

To study proteins induced by the adenovirus, AdTLRGDK was used, which expresses green fluorescent protein (GFP) under a genetic background similar to VCN-01 (Rodríguez-García et al., “Safety and Efficacy of VCN-01, an Oncolytic Adenovirus Combining Fiber HSG-Binding Domain Replacement with RGD and Hyaluronidase Expression,” Clinical Cancer Research 2015; 21(6):1406-18). Green fluorescent protein (GFP) transfected by AdTLRGDK was studied (Shiozawa et al., “Immunohistochemical analysis of the expression of cdk4 and p16INK4 in human endometrioid-type endometrial carcinoma,” Cancer 1997; 80(12):2250-6). In a first experiment, Y79 cells were arrested in the S phase with hydroxyurea (4 mM). After 24 h, cells were washed with phosphate buffered saline (PBS) and exposed to 50 MOI of AdTLRGDK. Fluorescence images of the cells at 24, 48 and 72 h post infection were acquired, and the pellets were collected for GFP analysis by flow cytometry (NovoCyte flow cytometry system, ACEA Biosciences, San Diego, CA, USA), using the auto-fluorescence of untreated cells to gate infected cells signal. Data was analyzed using the NovoExpress software (ACEA Biosciences). In a second cytometry experiment, Y79, RBT-5 and RBT-7 cells were infected with 50 MOI of AdTLRGDK. After 24 h, topotecan (2 μM), carboplatin (12.5 μM), melphalan (10 μM), hydroxyurea (100 μM) or fresh culture medium was added. After 24 h, the expression of GFP was quantified. To address the sequence of treatments on GFP expression, cells were exposed to topotecan (2 μM) or hydroxyurea (100 μM) 6 h before VCN-01 infection at 50 MOI and collected 48 h later.

Viral production assay. Y79 cells were infected with a concentration of 500 multiplicity of infection (MOI; i.e., transducing units of virus per cell), which was sufficient to achieve 80% to 100% infectivity. After 4 h, the cells were washed three times with PBS, fresh medium was added, and the cells were incubated with or without topotecan (2 μM). At time points 30, 48 and 72 h post-infection, the cell pellets were collected and exposed to three rounds of freeze-thaw lysis. Viral titers were determined in the pellets in triplicate, following an anti-hexon staining-based method in HEK293 cells (Cascallo et al., 2007).

Cell cycle. To address the effect of the chemotherapeutics on the cycle of retinoblastoma cells, we used hydroxyurea (100 μM) as a positive control of S-phase cell cycle arrest (Alvino et al., “Replication in hydroxyurea: it's a matter of time,” Mol Cell Biol 2007; 27(18):6396-406).

10⁶ retinoblastoma cells were treated with topotecan (2 μM), carboplatin (12.5 μM), melphalan (10 μM) or hydroxyurea (100 μM) for 24 h. Then, the cells were collected and fixed in 70% ethanol for 2 h. The propidium iodide (PI)-based assay FxCycle™ PI/RNase (Thermo Fisher Scientific) was used as staining solution prior to quantification by flow cytometry (NovoCyte).

To synchronize cells in the S phase of the cycle, 10⁶ cells were exposed to high concentration of hydroxyurea (4 mM). After 24 h, the cells were washed with PBS, fresh medium was added, and the cells were incubated for 2, 4, 8 and 24 h, until processing for cytometry.

Apoptosis. The apoptotic marker cPARP in retinoblastoma cells exposed to topotecan, VCN-01 or the combination was assessed.

10⁶ retinoblastoma cells were plated per well in 6-well plates. After 24 h, 50 MOI of VCN-01 was added. The next day, topotecan (2 μM) was added. The cells were collected after 24 h, washed twice with 1% of bovine serum albumin in PBS and fixed with 70% ethanol for 2 h at 4° C. The cells were labeled with primary antibody cPARP (5625S, 1:1000, Cell Signaling) and with secondary antibody (ab150077, 1:2000, Abcam) before flow cytometry (NovoCyte).

Accumulation of topotecan in VCN-01-infected retinoblastoma. The Animal Experimental Ethics Committee at the University of Barcelona (animal protocol number 482/18) approved animal experimentation. Retinoblastoma cells in the eyes of athymic nude mice (Envigo, Barcelona, Spain), as previously described (Pascual-Pasto et al., 2016). After 14 days, VCN-01 (3×10⁹ vp/eye) was inoculated. After another 14 days, s.c. osmotic pumps loaded with topotecan were implanted to achieve constant topotecan in plasma, as described previously (Pascual-Pasto et al., “Increased delivery of chemotherapy to the vitreous by inhibition of the blood-retinal barrier,” J Control Release 2017; 264:34-44), and collected tumor samples for analysis of topotecan.

Animal experiments were carried out following institutional and European guidelines (EU Directive 2010/63/EU) and the ARRIVE guidelines (van den Heuvel and Dyson, “Conserved functions of the pRB and E2F families,” Nature Reviews Molecular Cell Biology 2008; 9 (9): 713-24). Retinoblastoma cells (2×10⁵) were inoculated into the posterior segment of the eye of six week old athymic nude mice using a dull 33G needle, as previously described (Giani et al., 1994). After 14 days, a single dose of VCN-01 (3×10⁹ vp/eye) was inoculated. After another 14 days, an Alzet osmotic pump (2001D, Durect, Palo Alto, CA, USA) was implanted, loaded with 1 mg/ml of topotecan. These pumps released topotecan at a dose of 25 μg/h and reached constant levels in plasma of approximately 100 ng/ml. At the steady state (6 h), tumors and plasma were collected. Samples were analyzed by high performance liquid chromatography (HLPC) as described previously (Nevins, “The Rb/E2F pathway and cancer,” Human Molecular Genetics 2001; 10 (7): 699-703).

Intratumoral infection of VCN-01. S.c. tumors were established in athymic nude mice. Upon xenograft engraftment, mice received one intratumoral injection of VCN-01, alone or combined with one cycle of topotecan, carboplatin, or hydroxyurea. At 5 and 15 days after VCN-01 inoculation, genome copies of VCN-01 were quantified in the tumors and immunostained for E1A and E2F-1.

To establish s.c. tumors, 10⁶ retinoblastoma cells suspended in 25 UL matrigel (Corning, Glendale, AZ, USA) were injected in athymic nude mice. Upon xenograft engraftment (tumor volumes ranging 100-200 mm³), treatments were started. Mice received VCN-01 (3×10⁹ vp in 20 UL vehicle, single intratumoral injection at day 1), alone or combined with one cycle of topotecan (0.6 mg/kg, intraperitoneal -i.p.-, daily at days 1-5), carboplatin (40 mg/kg, i.p, one single dose at day 1), or hydroxyurea (200 mg/kg, i.p., daily at days 1-5). A group of mice received one intratumoral injection of the vehicle of VCN-01 (20 mM pH 8.0 tris buffer, 25 mM NaCl and 2.5% glycerol). Mice were sacrificed at 5 and 15 days after VCN-01 inoculation, the tumors were dissected and tumor homogenates were prepared by adding 10 μL of water per mg of tissue and homogenizing with a Bullet Blender turbine Storm 24 (Quasar instruments, Colorado Springs, CO, USA). In tumor homogenates, genome copies of VCN-01 were quantified. Part of the tumor was processed in 4% paraformaldehyde and embedded in paraffin for immunohistochemistry analysis. In 4 μm paraffin sections, hematoxylin and eosin (H&E) staining was performed and human nuclei (MAB4383, 1:200, Merck Millipore) were immunostained for viral protein E1A (ab33183, 1:200, Abcam) and E2F-1 (sc-251, 1:50, Santa Cruz Biotechnology).

Antitumor activity in retinoblastoma. For orthotopic intraocular engraftments 2×10⁵ retinoblastoma cells were inoculated per eye, in 2 μL matrigel (Pascual-Pasto et al., 2016). To obtain s.c. xenografts 10⁶ cells were injected in 25 μl matrigel. For ocular tumors, treatments were started at day 8 after inoculation (when tumors were appreciable by fundus examination). For s.c. tumors, treatments were started when volumes achieved 100-300 mm³ (day 1). After treatments, the survivors were followed up until endpoint and the median survival of each group was calculated, as described previously (Mandigo et al., 2022; Rodríguez-García et al., 2015).

In a first study, one dose of VCN-01 (3×10⁸ vp in 2 μL vehicle, intravitreal, day 15), therapeutic schedules of topotecan or standard of care (SoC) retinoblastoma chemotherapy, or their combinations, was administered in the Y79 intraocular xenograft. For topotecan, mice received 0.6 mg/kg i.p. injections on days 8-12 and 29-33. For SoC, mice received i.p carboplatin 40 mg/kg, on days 8 and 29, and etoposide 6 mg/kg, on days 8-10 and 29-31.

In a second experiment, the sequence of administration of one injection of VCN-01 and one 5-day cycle of topotecan was addressed. Mice with intraocular tumors (Y79 or RBT-7) received either topotecan 0.6 mg/kg, i.p. on days 8-12, one intravitreal injection of VCN-01 (3×10⁸ vp) on day 8, or the combination of topotecan and VCN-01. Y79-bearing mice also received a switched treatment sequence, with VCN-01 on day 12, after the last dose of topotecan.

In a third study, the number of cycles of topotecan (injections on days 8-12, 15-19, 29-33, 36-40, 50-54 and 57-61) was maximized and two intraocular injections of VCN-01 (3×10⁷ vp/eye) at days 8 and 15 to mice bearing RBT-2 xenografts were administered (Pascual-Pasto et al., 2019).

In a fourth study, to cancel the action of the blood-retinal barrier restraining drug delivery (Pascual-Pasto et al., “Therapeutic targeting of the RB1 pathway in retinoblastoma with the oncolytic adenovirus VCN-01,” Science Translational Medicine 2019; 11(476):eaat9321), mice were inoculated s.c. in both flanks with Y79 or RBT-7 cells. These animals were treated with either topotecan 0.6 mg/kg, i.p. on days 1-5, VCN-01 (3×10⁹ vp in 20 UL vehicle, intratumoral, day 1), or the combination in the same sequence.

Antitumor activity in CNS-disseminated retinoblastoma. A model of retinoblastoma dissemination was established in the leptomeninges of the CNS. Anesthetized mice were restrained in a stereotactic apparatus (Stoelting, Wood Dale, IL, USA). Using a mouse brain atlas (Paxinos and Franklin, “The mouse brain in stereotaxic coordinates,” San Diego: Academic Press; 2001), the coordinates of the fourth ventricle were located and the surface of the skull was drilled. Then, 10⁶ million cells (RBT-7) in 10 UL matrigel was injected with a dull 22G needle attached to a 50 UL syringe (Hamilton, Bonaduz, Switzerland). One week later, treatments were started with VCN-01 (3×10⁹ vp in 20 UL vehicle, intraventricular, day 8), topotecan (0.6 mg/kg, i.p., days 8-12), SoC chemotherapy (carboplatin, 40 mg/kg, i.p., day 8, and etoposide, 6 mg/kg, i.p., days 8-10) or the combination of VCN-01 and chemotherapies. The control group received the vehicle of VCN-01 (intraventricular). Animals achieved experimental endpoints upon deterioration of condition or 20% weight loss. Brains collected at endpoint were embedded in paraffin and stained with hematoxylin and eosin (H&E), human nuclei (MAB4383, 1:200, Millipore), viral protein E1A (ab33183, 1:200, Abcam) and CD45 (leukocyte common antigen; 70257, 1:200, Cell Signaling).

In a second experiment, 10⁶ cells were injected following the same procedure in 12 mice. After one week, the mice were treated with local injections of vehicle, VCN-01 (3×10⁹ vp), systemic topotecan or the combination. The mice were sacrificed 2 h after the last dose of topotecan. Brain homogenates were prepared by adding 10 μL of water per mg of tissue. DNA, protein and mRNA was extracted as previously described for s.c. tumors. Part of the tumor was processed for immunohistochemistry analysis.

Antitumor activity in extracranial pediatric solid tumors. 5-10 mm³ fresh fragments of Ewing sarcoma or neuroblastoma PDX were implanted in both flanks of mice. Upon s.c. engraftment (tumor volume 150-200 mm³), mice received topotecan (0.6 mg/kg, i.p., days 1-5), VCN-01 (3×10⁹ vp in 20 UL vehicle, intratumoral, day 1), or the combination. In two additional groups, an alternative topoisomerase 1 inhibitor, irinotecan (10 mg/kg, i.p., on days 1-5) we used in place of topotecan. Upon completion of treatments, tumor volumes were followed until endpoint (1500 mm³), or until day 80.

Statistics. The software GraphPad Prism 8 (La Jolla, CA, USA) and R Core Team (R Foundation for Statistical Computing, Vienna, Austria) was used for statistical analyses and graphical representation of the data.

At least three replicates for each condition in vitro were performed. To compare means of two different groups, the t test or the Mann-Whitney test was used. For more than two groups, the ANOVA test or the Kruskal-Wallis test with Dunn's correction was used. Median survivals were calculated using the Kaplan-Meier method and compared using the log-rank test with the Bonferroni correction for multiple comparisons. In most studies, all groups were compared with the control (vehicle) group. In the study of the CNS-disseminated retinoblastoma, all groups were compared with the one receiving the combination of VCN-01 and topotecan. For all the analyses, P<0.05 was defined as statistical significance.

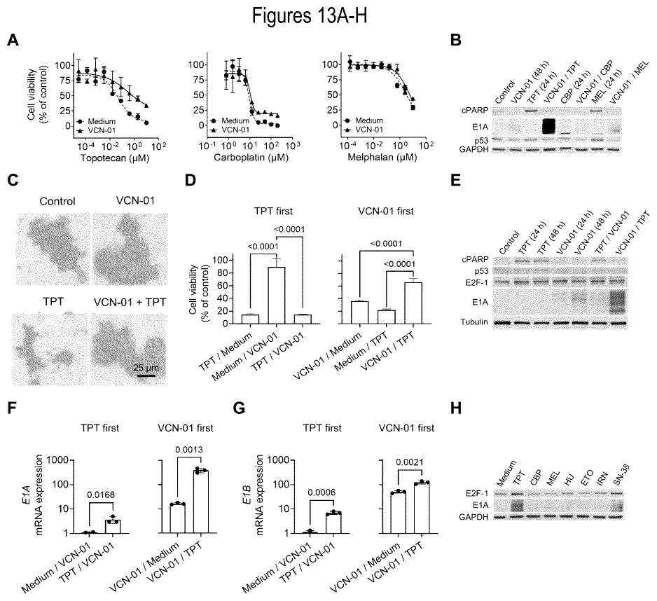

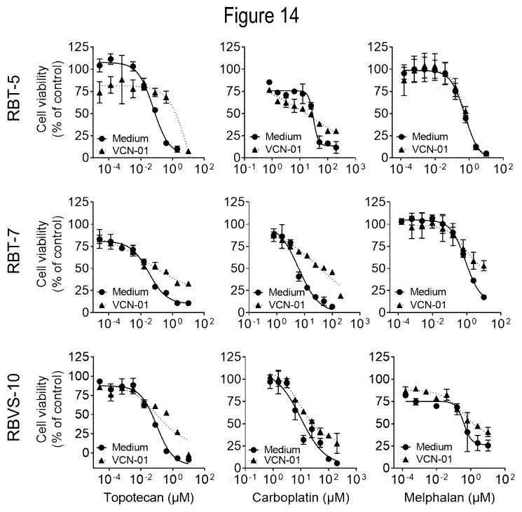

Interaction of VCN-01 and chemotherapeutics in retinoblastoma: apoptosis and infection. Infection of retinoblastoma cells with VCN-01 decreased cell sensitivity to topotecan, carboplatin and melphalan (FIG. 13A and FIG. 14) and increased the concentration of drugs required for a 50% decrease in cell proliferation (Table 2).

TABLE 2
Susceptibility of retinoblastoma cells to VCN-01, chemotherapy agents or the combinations in vitro.

| Cell | | | | | | | |
|---|---|---|---|---|---|---|---|
| model | VCN-01¹ | Topotecan (μM)³ | VCN-01 | Carboplatin (μM) | VCN-01 | Melphalan (μM) | VCN-01 |
| ID | (MOI)² | Single agent | (10 MOI) | Single agent | (10 MOI) | Single agent | (10 MOI) |
| HSJD- | 8.26 | 0.00738 | 0.0507 | n.p. | n.p. | n.p. | n.p. |
| RBT-2 | (5.72- | (0.00449- | (0.0228- | | | | |
| | 11.9) | 0.0122) | 0.118) | | | | |
| HSJD- | 0.0865 | 0.0879 | 0.586 | 19.0 | 15.5 | 0.478 | 0.636 |
| RBT-5 | (0.0572- | (0.0669- | (0.166- | (13.4- | (12.6- | (0.307- | (0.375- |
| | 0.131) | 0.116) | 3.47) | 26.6) | 19.1) | 0.843) | 1.33) |
| HSJD- | 6.77 | 0.0151 | 0.118 | 6.21 | 36.8 | 1.42 | 8.48 |
| RBT-7 | (4.13- | (0.00996- | (0.0713- | (5.22- | (29.7- | (1.14- | (4.11- |
| | 11.1) | 0.0228) | 0.200) | 7.41) | 46.2) | 1.83) | 27.1) |
| HSJD- | 4.95 | 0.0296 | 0.126 | 13.6 | 38.5 | 0.241 | 2.02 |
| RBVS-10 | (3.29- | (0.0191- | (0.0605- | (10.3- | (30.1- | (0.0812- | (1.26- |
| | 7.45) | 0.0449) | 0.265) | 18.1) | 49.9) | 0.683) | 3.51) |
| Y79 | 0.0814 | 0.0303 | 0.893 | 9.27 | 10.3 | 3.25 | 5.83 |
| | (0.0648- | (0.0165- | (0.243- | (8.09- | (7.74- | (2.23- | (4.21- |
| | 0.102) | 0.0557) | 3.28) | 10.6) | 13.8) | 4.74) | 8.07) |

¹The activity of VCN-01 on retinoblastoma models was previously published in [Pascual-Pastó et al, 2019].
²Values are expressed as the half maximal inhibitory concentration (IC50; MOI), i.e., the concentration of the anticancer agent required to cause a reduction of 50% in cell viability. Values within brackets indicate the 95% confidence interval.
³Values are the IC50 (μM) with 95% confidence internals.
n.p., not performed

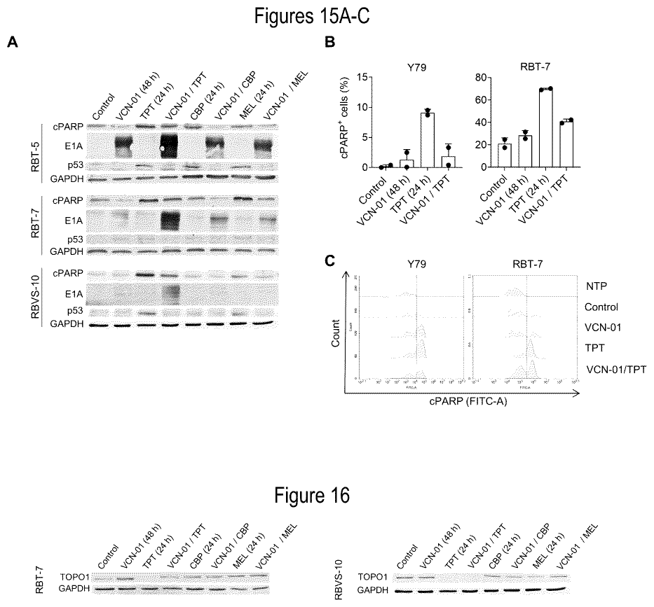

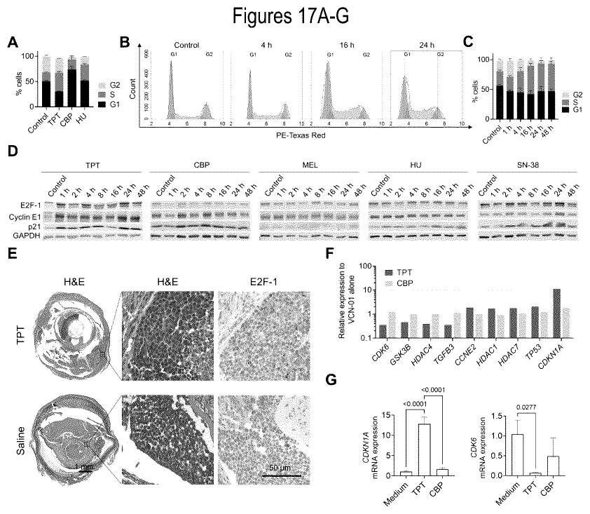

Viral infection impeded the expected increment of proteins triggered by the DNA damage induced by cytotoxic agents, such as cPARP and p53 (FIG. 13B and FIG. 15A-C). Unexpectedly, early viral protein E1A was highly overexpressed in the infected cells treated with topotecan (FIG. 13B and FIG. 15A). Infected cells conserved their shape and integrity during the early infection times (48 h), even in the presence of topotecan (FIG. 13C). Cells pre-exposed to topotecan before VCN-01 infection were sensitive to the antiproliferative activity of the drug, regardless the presence of the virus at a later stage (FIG. 13D). In contrast, previous infection partially abolished the activity of the drug (FIG. 13D). Topotecan pretreatment inhibited the posterior infection of cells by VCN-01, leading to low expression of E1A (protein and gene), accumulation of E2F-1 and increased expression of proapoptotic proteins p53 and cPARP (FIG. 13E,F). In contrast, topotecan addition after the virus boosted up to two orders of magnitude the expression of E1A (FIG. 13E,F) and E1B (FIG. 13G). Another camptothecin, SN-38, increased the expression of E2F-1 and E1A similarly to topotecan in cells pre-exposed to VCN-01, in contrast to the alkylating drugs, the antimetabolite drug hydroxyurea and the topoisomerase II inhibitor etoposide, which did not modify the levels of these proteins (FIG. 13H). The molecular target of camptothecins, topoisomerase 1, increased upon VCN-01 infection in one primary culture and remained unchanged in another (FIG. 16). Overall, the results indicate that adenoviral infection impedes the apoptotic action of topotecan, while topotecan favors the expression of early viral proteins in cells pre-exposed to VCN-01. Without wishing to be bound by theory, it is thought that this is by increasing the expression of the viral promoter E2F-1 in cells.

Interaction of VCN-01 and chemotherapeutics: cell cycle and E2F-1 expression. Because cells in S-phase are more suitable to infection by adenoviruses, cell cycle changes were addressed in retinoblastoma cells exposed to the chemotherapeutics used for the standard of care of this disease. Only topotecan increased the percentage of cells in S-phase, similar to the positive control hydroxyurea (Alvino et al., 2007) (FIG. 17A). The effect of topotecan was appreciable early and lasted for at least 48 h (FIG. 17B,C). Topotecan, but not carboplatin, melphalan or hydroxyurea promoted cellular events consistent with G1/S-phase cell cycle progression, such as increased protein expression of E2F-1, p21 and cyclin E1, which were appreciable 1 h after exposure, for at least 48 h (FIG. 17D). SN-38 produced similar effects, although they appeared slightly later than with topotecan (FIG. 17D). In mice bearing intraocular Y79 tumors, the expression of E2F-1 in cancer cells was higher in the animals treated with topotecan, compared to that found in untreated animals (FIG. 17E).

In a primary retinoblastoma pre-infected with VCN-01, a gene expression array revealed that topotecan upregulated genes that promote S-phase arrest during the cell cycle, with a 10-fold increase in the expression of the gene CDKN1A (encoding for p21 protein) (FIG. 17F). Conversely, it downregulated genes that promote G1/S transition, with a 15-fold decrease in the expression of CDK6 (FIG. 17F). The array findings were confirmed using qPCR (FIG. 17G).

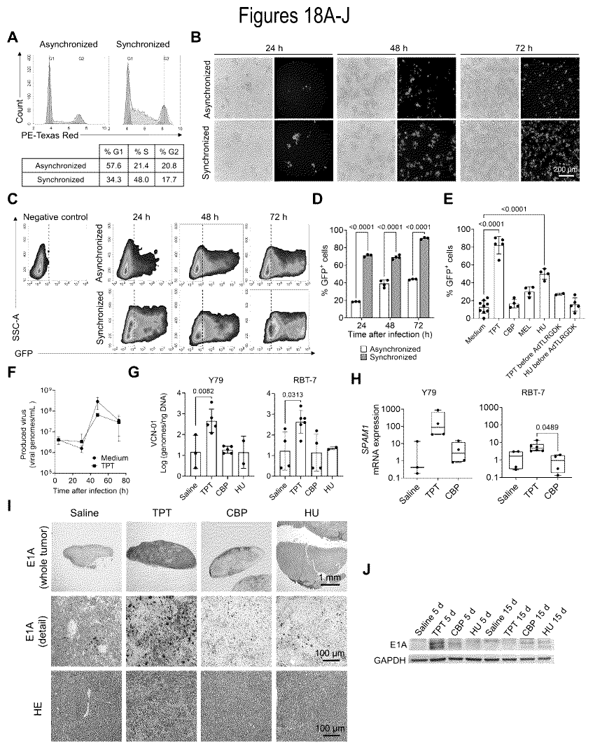

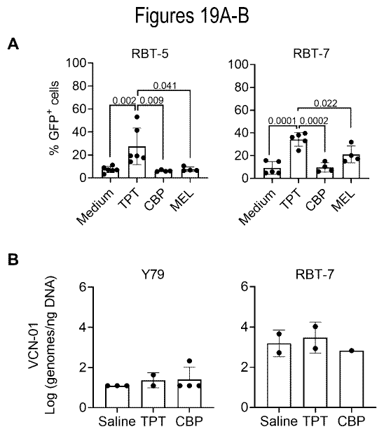

Interaction of VCN-01 and chemotherapeutics: gene transduction and viral replication. To address whether S-phase cell cycle arrest favored the expression of viral genes, Y79 cells were synchronized in the S-phase using hydroxyurea at high concentration (FIG. 18A). After washing steps, treatment of the synchronized cells with the adenovirus AdTLRGDK significantly increased transgene expression (GFP) (FIG. 18B-D). After 72 h, 44% of the asynchronized cells were infected, in contrast to 90% of the synchronized ones (FIG. 18D). In Y79 cells and primary retinoblastoma cultures pre-infected with the adenovirus AdTLRGDK, topotecan and hydroxyurea increased transgene expression, in contrast to carboplatin and melphalan (FIG. 18E and FIG. 19A). The reverse sequence of treatments (i.e., drug exposure before viral infection) did not enhance viral transduction (FIG. 18E). Topotecan did not increase viral production in Y79 cells pre-infected with a high viral load of 500 MOI (FIG. 18F). Following one intratumoral injection of VCN-01 in s.c. xenografts, subsequent treatment with systemic topotecan resulted in higher counts of viral genomes (FIG. 18G) and higher expression of recombinant hyaluronidase after 5 days, compared to carboplatin treatment (FIG. 18H). Different counts of viral genomes were not observed after 15 days (FIG. 19B). After 5-day topotecan treatment, tumors pretreated with VCN-01 had the highest number of E1A-positive cells (FIG. 18I) and the highest level of E1A protein expression (FIG. 18J), compared to tumors treated with saline, carboplatin or hydroxyurea.

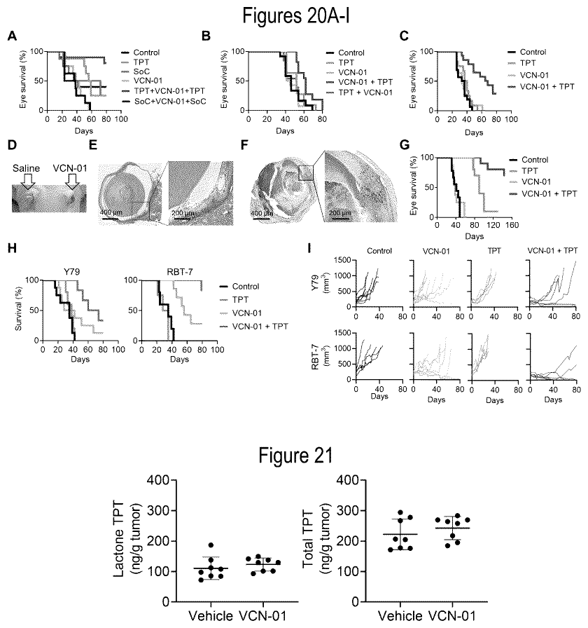

Activity of VCN-01 and topotecan in retinoblastoma xenografts. Because long term exposures to VCN-01 and chemotherapy at clinically relevant concentrations are not feasible in vitro, in vivo experiments were performed to address the therapeutic effect of the treatment combinations. One intraocular dose of VCN-01 administered between two cycles of topotecan prolonged the median ocular survival of Y79 xenografts longer than 80 days, which was a significant therapeutic benefit compared to untreated control eyes (37 days; P=0.0005). Treatments with topotecan or with the standard of care (carboplatin and etoposide) resulted in modest improvements in median survival (58 and 55 days, respectively), although not significant compared to controls (P=0.062 and P=0.674, respectively) (FIG. 20A). As a single agent, the selected dose of VCN-01 was sub-therapeutic (40 days, P=1) (FIG. 20A). The sequence of administration producing a better-than-additive efficacy was the one in which VCN-01 was administered first, followed by topotecan (FIG. 20B). This therapeutic sequence achieved a median ocular survival of 62 days, significantly longer than controls (46 days, P=0.0436). The reverse sequence, topotecan followed by VCN-01, did not result in therapeutic benefit (52 days, P=1) (FIG. 20B). The synergism of VCN-01 and topotecan was confirmed in one primary retinoblastoma xenograft that achieved a median survival of 67 days, longer than controls (35 days, P<0.0001), while VCN-01 and topotecan were not effective as single agents (33 days and 39 days, respectively) (FIG. 20C-E). In tumors treated with the combination areas of active VCN-01 infection at endpoint (i.e., at the time of enucleation due to tumor progression) were detected, even at day 65 after one single VCN-01 injection (FIG. 20F). Maximization of the number of cycles of topotecan and VCN-01 injections resulted in significant extension of the median ocular survival (FIG. 20G; P<0.0001). Biodistribution of topotecan in intraocular tumors did not change upon concomitant treatment with VCN-01 (FIG. 21). The superior therapeutic activity of the combination treatment was confirmed in two s.c. retinoblastoma xenografts achieving 67 and more than 80 days of median survival for Y79 and RBT-7, respectively, significantly longer than group control (35 days, P=0.0009, and 35 days, P=0.0021, respectively) (FIG. 20H, I).

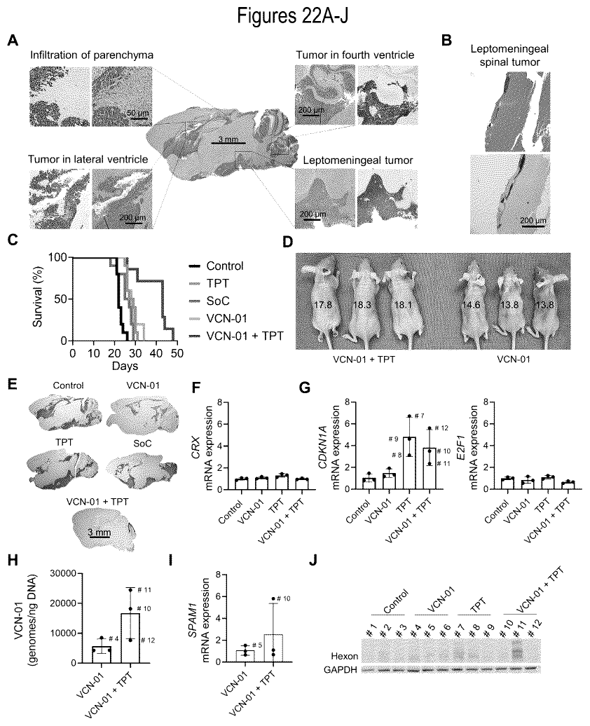

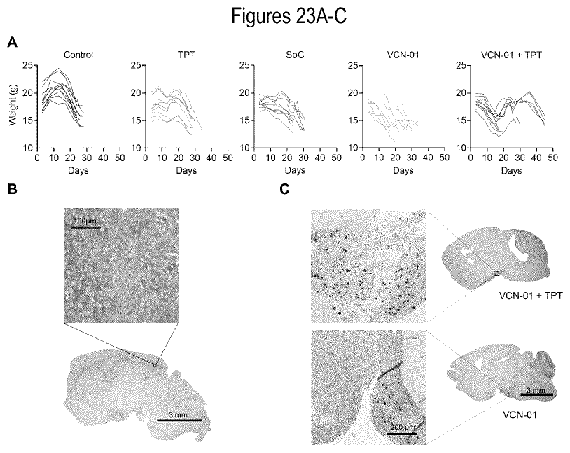

Activity of VCN-01 and topotecan in CNS-disseminated retinoblastoma. The mouse model of CNS-disseminated retinoblastoma occupied and enlarged the whole leptomeningeal space, including the meninges surrounding the spinal cord (FIG. 22A, B). To a lesser extent, tumor cells invaded the brain parenchyma (FIG. 22A). In the absence of treatment, median survival was 22 days (FIG. 22C). Mice treated with the combination of intraventricular VCN-01 and systemic topotecan achieved a median survival of 43 days, significantly longer compared to control (P=0.0004), topotecan (28 days, P=0.0072), standard of care (26 days, P=0.0040) and VCN-01 (28 days, P=0.0372) (FIG. 22C). At the selected dose of intraventricular VCN-01, relevant toxicity (acute weight loss) was observed (FIG. 23A). Due to toxicity, 50% animals of the VCN-01 group, 30% of the VCN-01 and topotecan combination, and 70% of the combination of VCN-01 and standard of care died on days 7-9 after treatment, likely because of brain inflammation. In autopsies, areas of lymphocyte infiltration in the brain were observed (FIG. 23B). For the survival analyses, animals dying of toxicity and the whole group in which more than 50% of the animals had lethal toxicity were excluded. To mitigate brain inflammation observed during the first days of the experiment, dexamethasone was empirically administered (5 mg/kg, i.p., daily for 5 consecutively days) to all mice, starting at day 15. Survivors recovered from treatment and gained weight (FIG. 23A). Animals treated with VCN-01 and topotecan experienced less acute toxicity and recovered completely (FIG. 22D). Among the animals sacrificed at day 26 to study tumor burden, the one treated with VCN-01 and topotecan presented less tumor burden than the rest (FIG. 22E).

Among the mice sacrificed at the last day of treatment, all had similar intracerebral load of retinoblastoma cells (FIG. 22F). Brain homogenates from mice treated with systemic topotecan, administered alone or after intraventricular VCN-01, had the highest expression of CDKN1A (FIG. 22G). Levels of E2F1 expression did not vary among groups (FIG. 22G). The number of E1A-positive cells was higher in mice treated with the combination, compared to mice treated with intraventricular VCN-01 alone (FIG. 23C). Brain homogenates from the group treated with the combination of VCN-01 and topotecan presented the highest counts of VCN-01 genomes (FIG. 22H), the highest expression of human hyaluronidase (FIG. 22I) and the highest positivity for viral hexons (FIG. 22J).

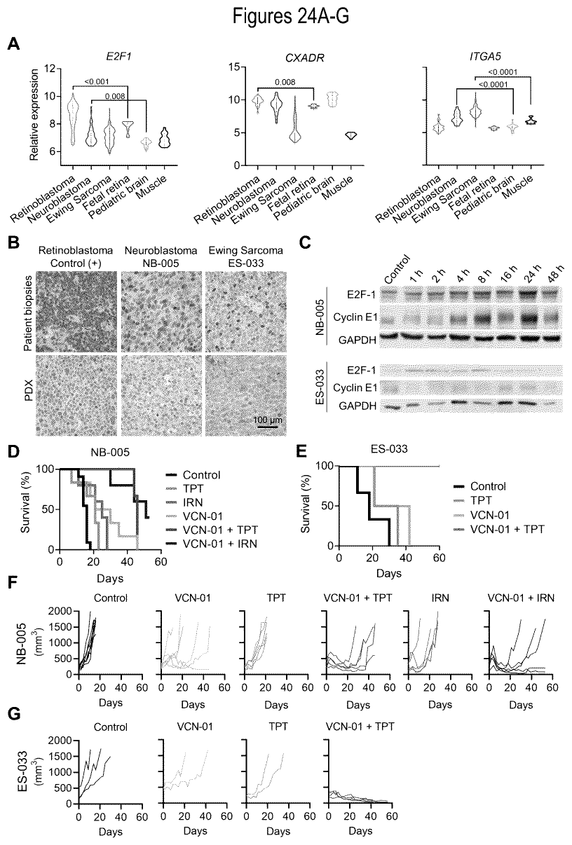

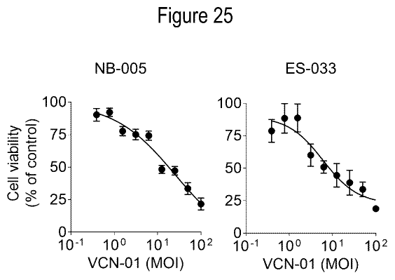

Activity of VCN-01 and topotecan in Ewing sarcoma and neuroblastoma. First, the gene expression public databases GSE16237 (Ohtaki et al., “A robust method for estimating gene expression states using Affymetrix microarray probe level data,” BMC Bioinformatics 2010; 11:183-83) and GSE34620 (Postel-Vinay et al., “Common variants near TARDBP and EGR2 are associated with susceptibility to Ewing sarcoma,” Nature Genetics 2012; 44 (3): 323-27) were used to verify that Ewing sarcoma and neuroblastoma express E2F1 and the adenoviral receptors CXADR and ITGA5 (FIG. 24A). E2F-1 protein expression was positive in biopsies and the corresponding PDX of one patient each with Ewing sarcoma and neuroblastoma (FIG. 24B). In vitro, overexpression of E2F-1 and cyclin E1 after topotecan exposure was observed (FIG. 24C). VCN-01 inhibited the proliferation of PDX-derived primary cells (FIG. 25). Both s.c. xenografts were highly resistant to single treatment with topotecan, irinotecan or VCN-01 (FIG. 24D-G). In the neuroblastoma PDX, the combination of VCN-01 with topotecan or irinotecan provided significant survival benefit (P=0.0050 and P=0.0002, respectively) and inhibition of tumor growth, compared to controls (FIG. 24D,F). In the Ewing sarcoma PDX, the combination of VCN-01 and topotecan achieved a better-than-additive therapeutic activity in animal survival (P=0.0303, compared to controls) and tumor growth (FIG. 24E,G).

### Discussion:

In summary, this Example demonstrates, inter alia, the synergistic antitumor activity of VCN-01 and camptothecins in highly aggressive and chemorefractory pediatric solid tumors expressing E2F-1. Without wishing to be bound by theory, it is thought that the molecular data support that E2F-1 accumulation and stabilization upon DNA damage produced by topotecan leads to an increase in the selectivity and infectivity of VCN-01. This work is widely applicable to improve the oncolytic activity of viruses containing E2F-1 promoters and supports the development of clinical trials.

### EQUIVALENTS

While the disclosure has been described in connection with specific embodiments thereof, it will be understood that it is capable of further modifications and this application is intended to cover any variations, uses, or adaptations of the disclosure following, in general, the principles of the disclosure and including such departures from the present disclosure as come within known or customary practice within the art to which the disclosure pertains and as may be applied to the essential features hereinbefore set forth and as follows in the scope of the appended claims.

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present disclosure is not entitled to antedate such publication by virtue of prior disclosure.

As used herein, all headings are simply for organization and are not intended to limit the disclosure in any manner. The content of any individual section may be equally applicable to all sections.

### EMBODIMENTS

Various additional embodiments of the disclosure are provided by the following enumerated embodiments, which can be combined in any number and in any combination.

Embodiment 1. A method for treating retinoblastoma, Ewing sarcoma, or neuroblastoma in a patient in need thereof, comprising co-administering to the patient (i) an oncolytic adenovirus comprising replication machinery specific for tumor cells and, optionally, a polynucleotide sequence encoding a hyaluronidase enzyme inserted into its genome and (ii) a topoisomerase I inhibitor or a prodrug thereof.

Embodiment 2. The method according to embodiment 1, wherein the oncolytic adenovirus is administered by intraocular, intravenous, intraarterial, intrathecal, intravitreal, or intratumoral injection.

Embodiment 3. The method according to embodiment 1 or 2, wherein the topoisomerase I inhibitor or a prodrug thereof is administered intravenously, intraarterially, intravitreally, intrathecally, intraventricularly, or orally.

Embodiment 4. The method according to any one of embodiments 1-3, wherein the oncolytic adenovirus and the topoisomerase I inhibitor or a prodrug thereof are formulated in separate compositions.

Embodiment 5. The method according to any one of embodiments 1-3, wherein the oncolytic adenovirus and the topoisomerase I inhibitor or a prodrug thereof are formulated in a single composition.

Embodiment 6. The method according to any one of embodiments 1-5, wherein the separate compositions are administered simultaneously or contemporaneously.

Embodiment 7. The method according to any one of embodiments 1-6, wherein the oncolytic adenovirus is administered first and the topoisomerase I inhibitor or a prodrug thereof is administered within about 60 minutes of the administration of the oncolytic adenovirus.

Embodiment 8. The method of embodiment 7, wherein the topoisomerase I inhibitor or a prodrug thereof is administered within about 30 minutes, within about 20 minutes, within about 10 minutes, within about 5 minutes, or within about 1 minute of the administration of the oncolytic adenovirus.

Embodiment 9. The method according to any one of embodiments 1-8, wherein the subject is administered one intrathecal injection of the oncolytic adenovirus concomitant with systemic or intrathecal cycles of the topoisomerase I inhibitor or prodrug thereof.

Embodiment 10. The method according to any one of embodiments 1-9, wherein the subject is administered the oncolytic adenovirus and the topoisomerase I inhibitor or prodrug thereof systemically.

Embodiment 11. The method according to any one of embodiments 1-10, wherein the oncolytic adenovirus and/or topoisomerase I inhibitor or prodrug thereof is co-administered with one or more local and/or systemic corticosteroids, optionally wherein the one or more local and/or systemic corticosteroids are selected from hydrocortisone, prednisone, prednisolone, methylprednisolone, dexamethasone, betamethasone, alsosterone, budesonide, fluticasone, flunisolide, ciclesonide, mometasone, beclomethasone, triamcinolone, and tixocortol.

Embodiment 12. The method according to any one of embodiments 1-11, wherein the topoisomerase I inhibitor or prodrug thereof is topotecan, SN-38, or irinotecan.

Embodiment 13. The method according to any one of embodiments 1-12, wherein the hyaluronidase enzyme is the human hyaluronidase enzyme PH20.

Embodiment 14. The method according to embodiment 13, wherein the sequence that encodes a hyaluronidase enzyme is SEQ ID NO: 9, or a sequence having at least about 75%, at least about 80%, at least about 85%, at least about 90%, at least about 95%, at least about 98%, or at least about 99% sequence identity thereto.

Embodiment 15. The method according to any one of embodiments 1-14 wherein the oncolytic adenovirus is generated from a human adenovirus serotype 5.

Embodiment 16. The method according to any one of embodiments 1-15, wherein the treatment is of retinoblastoma and oncolytic adenoviral replication occurs in tumor cells with an aberrant Rb-E2F pathway and not in healthy, non-tumor, or normal cells.

Embodiment 17. The method according to embodiment 16, wherein the oncolytic adenovirus is engineered to replicate in tumor cells and not healthy, non-tumor, or normal cells by deletion Δ24 in the sequence coding for the E1a protein and the insertion of four binding sites to E2F-1 and one binding site to Sp1 into the endogenous promoter of E1a to control the expression of E1a.

Embodiment 18. The method according to any one of embodiments 1-17, wherein the oncolytic adenovirus has the capsid modified such that the heparan sulfate binding domain ⁹¹KKTK⁹⁴ (SEQ ID NO: 8) present in the adenovirus fiber has been replaced, optionally by the domain ⁹¹RGDK⁹⁴ (SEQ ID NO: 9).

Embodiment 19. The method according to any one of embodiments 1-18, wherein the oncolytic adenovirus is VCN-01 (SEQ ID NO: 3).

Embodiment 20. The method according to any one of embodiments 1-19, wherein the patient is a human patient.

Embodiment 21. The method according to embodiment 20, wherein the human patient is a pediatric human patient.

Embodiment 22. The method according to any one of embodiments 1-21, wherein the retinoblastoma, Ewing sarcoma, or neuroblastoma is a retinoblastoma, Ewing sarcoma, or neuroblastoma resistant to conventional chemotherapy and/or radiotherapy treatment.

Embodiment 23. The method according to any one of embodiments 1-22, wherein the method improves and/or increases and/or enhances antitumor efficacy compared to treatment with the topoisomerase I inhibitor or a prodrug thereof without the oncolytic adenovirus.

Embodiment 24. The method according to any one of embodiments 1-23, wherein the method results in reduced or maintained tumor size, and/or prevention or reduction of metastasis, secondary malignancy, or trilateral retinoblastoma associated with retinoblastoma, compared to treatment with the topoisomerase I inhibitor or a prodrug thereof without the oncolytic adenovirus.

Embodiment 25. The method according to any one of embodiments 1-23, wherein the method results in reduced or maintained tumor size, and/or prevention or reduction of metastasis, secondary malignancy, or recurrent disease associated with Ewing sarcoma, compared to treatment with the topoisomerase I inhibitor or a prodrug thereof without the oncolytic adenovirus.

Embodiment 26. The method according to any one of embodiments 1-23, wherein the method results in reduced or maintained tumor size, and/or prevention or reduction of metastasis, secondary malignancy, or recurrent disease associated with neuroblastoma, compared to treatment with the topoisomerase I inhibitor or a prodrug thereof without the oncolytic adenovirus.

Embodiment 27. The method according to any one of embodiments 1-26, wherein administration of the oncolytic adenovirus reduces apoptotic action of topotecan as compared to monotherapy.

Embodiment 28. The method according to any one of embodiments 1-27, wherein administration of topotecan following administration of the oncolytic adenovirus leads to S-phase cell cycle arrest.

Embodiment 29. The method according to any one of embodiments 1-28, wherein the S-phase cell cycle arrest results in increased infectivity of the oncolytic adenovirus as compared to treatment with the oncolytic adenovirus without the topoisomerase I inhibitor or prodrug thereof.

Embodiment 30. The method according to any one of embodiments 1-29, wherein administration of topotecan following administration of the oncolytic adenovirus leads to increased E2F-1, p21 and/or cyclin E1 expression as compared to treatment with the oncolytic adenovirus without the topoisomerase I inhibitor or prodrug thereof.

Embodiment 31. The method according to embodiment 30, wherein the increased E2F-1 expression results in increased oncolytic activity of the oncolytic adenovirus as compared to treatment with the oncolytic adenovirus without the topoisomerase I inhibitor or prodrug thereof.

Embodiment 32. The method according to any one of embodiments 1-31, wherein the increased infectivity and oncolytic activity of the oncolytic adenovirus occurs without substantially increasing replication of the oncolytic adenovirus.

Embodiment 33. The method according to any one of embodiments 1-32, wherein sequential treatments in which the oncolytic adenovirus is administered first, followed by systemic topotecan, enhance or increase cell infection and/or anticancer efficacy of the oncolytic adenovirus.

Embodiment 34. A method for treating retinoblastoma, Ewing sarcoma, or neuroblastoma in a patient in need thereof, comprising administering to the patient (i) an oncolytic adenovirus comprising replication machinery specific for tumor cells and, optionally, a polynucleotide sequence encoding a hyaluronidase enzyme inserted into its genome and (ii) a topoisomerase I inhibitor or a prodrug thereof.

Embodiment 35. The method according to embodiment 34, wherein the oncolytic adenovirus is administered by intraocular, intravenous, intraarterial, intrathecal, intravitreal, or intratumoral injection.

Embodiment 36. The method according to embodiment 34 or 35, wherein the topoisomerase I inhibitor or a prodrug thereof is administered intravenously, intravitreally, intraarterially, intrathecally, intraventricularly, or orally.

Embodiment 37. The method according to any one of embodiments 34-36, wherein the oncolytic adenovirus and the topoisomerase I inhibitor or a prodrug thereof are formulated in separate compositions.

Embodiment 38. The method according to any one of embodiments 34-37, wherein the oncolytic adenovirus and the topoisomerase I inhibitor or a prodrug thereof are formulated in a single composition.

Embodiment 39. The method according to any one of embodiments 34-38, wherein the oncolytic adenovirus is administered first and the topoisomerase I inhibitor or a prodrug thereof is administered within about 12 weeks of the administration of the oncolytic adenovirus.

Embodiment 40. The method of embodiment 39, wherein the topoisomerase I inhibitor or a prodrug thereof is administered within about 60 minutes, about 2 hours, about 3 hours, about 4 hours, about 5 hours, about 6 hours, about 7 hours, about 8 hours, about 9 hours, about 10 hours, about 11 hours, about 12 hours, about 1 day, about 2 days, about 3 days, about 4 days, about 5 days, about 6 days, about 7 days, about 8 days, about 9 days, about 10 days, about 11 days, about 12 days, about 13 days, about 2 weeks, about 3 weeks, about 4 weeks, about 5 weeks, about 6 weeks, about 7 weeks, about 8 weeks, about 9 weeks, about 10 weeks, or about 11 weeks of the administration of the oncolytic adenovirus.

Embodiment 41. The method according to any one of embodiments 1-32, wherein the subject is administered one intrathecal injection of the oncolytic adenovirus concomitant with systemic or intrathecal cycles of the topoisomerase I inhibitor or prodrug thereof.

Embodiment 42. The method according to any one of embodiments 1-32, wherein the subject is administered the oncolytic adenovirus and the topoisomerase I inhibitor or prodrug thereof systemically.

Embodiment 43. The method according to any one of embodiments 1-32, wherein the oncolytic adenovirus and/or topoisomerase I inhibitor or prodrug thereof is co-administered with one or more local and/or systemic corticosteroids, optionally wherein the one or more local and/or systemic corticosteroids are selected from hydrocortisone, prednisone, prednisolone, methylprednisolone, dexamethasone, betamethasone, alsosterone, budesonide, fluticasone, flunisolide, ciclesonide, mometasone, beclomethasone, triamcinolone, and tixocortol.

Embodiment 44. The method according to any one of embodiments 34-43, wherein the topoisomerase I inhibitor is topotecan or SN-38 and the topoisomerase I inhibitor prodrug is irinotecan.

Embodiment 45. The method according to any one of embodiments 34-44, wherein the hyaluronidase enzyme is the human hyaluronidase enzyme PH20.

Embodiment 46. The method according to embodiment 45, wherein the sequence that encodes a hyaluronidase enzyme is SEQ ID NO: 9, or a sequence having at least about 75%, at least about 80%, at least about 85%, at least about 90%, at least about 95%, at least about 98%, or at least about 99% sequence identity thereto.

Embodiment 47. The method according to any one of embodiments 34-46, wherein the oncolytic adenovirus is generated from a human adenovirus serotype 5.

Embodiment 48. The method according to any one of embodiments 34-47, wherein oncolytic adenoviral replication occurs in tumor cells with an aberrant Rb-E2F pathway and not in healthy, non-tumor, or normal cells.

Embodiment 49. The method according to embodiment 48, wherein oncolytic adenovirus is engineered to replicate in tumor cells and not healthy, non-tumor, or normal cells by deletion of a Rb binding domain the deletion Δ24 in the sequence coding for the E1a protein and the insertion of four binding sites to E2F-1 and one binding site to Sp1 into the endogenous promoter of E1a to control the expression of E1a.

Embodiment 50. The method according to any one of embodiments 34-49, wherein the oncolytic adenovirus has the capsid modified such that the heparan sulfate binding domain ⁹¹KKTK⁹⁴ (SEQ ID NO: 8) present in the adenovirus fiber has been replaced, optionally by the domain ⁹¹RGDK⁹⁴ (SEQ ID NO: 9).

Embodiment 51. The method according to any one of embodiments 34-50, wherein the oncolytic adenovirus is VCN-01 (SEQ ID NO: 3).

Embodiment 52. The method according to any one of embodiments 34-51, wherein the patient is a human patient.

Embodiment 53. The method according to embodiment 52, wherein the human patient is a pediatric human patient.

Embodiment 54. The method according to any one of embodiments 34-53, wherein the retinoblastoma, Ewing sarcoma, or neuroblastoma is a retinoblastoma, Ewing sarcoma, or neuroblastoma resistant to conventional chemotherapy and/or radiotherapy treatment.

Embodiment 55. The method according to any one of embodiments 34-54, wherein the method improves and/or increases and/or enhances antitumor efficacy compared to treatment with the topoisomerase I inhibitor or a prodrug thereof without the oncolytic adenovirus.

Embodiment 56. The method according to any one of embodiments 34-55, wherein the method results in reduced or maintained tumor size, and/or prevention or reduction of metastasis, secondary malignancy, or trilateral retinoblastoma associated with retinoblastoma, compared to treatment with the topoisomerase I inhibitor or a prodrug thereof without the oncolytic adenovirus.

Embodiment 57. The method according to any one of embodiments 34-55, wherein the method results in reduced or maintained tumor size, and/or prevention or reduction of metastasis, secondary malignancy, or recurrent disease associated with Ewing sarcoma, compared to treatment with the topoisomerase I inhibitor or a prodrug thereof without the oncolytic adenovirus.

Embodiment 58. The method according to any one of embodiments 34-55, wherein the method results in reduced or maintained tumor size, and/or prevention or reduction of metastasis, secondary malignancy, or recurrent disease associated with neuroblastoma, compared to compared to treatment with the topoisomerase I inhibitor or a prodrug thereof without the oncolytic adenovirus.

Embodiment 59. The method according to any one of embodiments 34-58, wherein administration of the oncolytic adenovirus reduces apoptotic action of topotecan as compared to monotherapy.

Embodiment 60. The method according to any one of embodiments 34-59, wherein administration of topotecan following administration of the oncolytic adenovirus leads to S-phase cell cycle arrest.

Embodiment 61. The method according to embodiments 60, wherein the S-phase cell cycle arrest results in increased infectivity of the oncolytic adenovirus as compared to treatment with the oncolytic adenovirus without the topoisomerase I inhibitor or prodrug thereof.

Embodiment 62. The method according to any one of embodiments 34-61, wherein administration of topotecan following administration of the oncolytic adenovirus leads to increased E2F-1, p21 and/or cyclin E1 expression as compared to treatment with the oncolytic adenovirus without the topoisomerase I inhibitor or prodrug thereof.

Embodiment 63. The method according to embodiment 62, wherein the increased E2F-1 expression results in increased oncolytic activity of the oncolytic adenovirus as compared to treatment with the oncolytic adenovirus without the topoisomerase I inhibitor or prodrug thereof.

Embodiment 64. The method according to any one of embodiments 34-63, wherein the increased infectivity and oncolytic activity of the oncolytic adenovirus occurs without substantially increasing replication of the oncolytic adenovirus.

Embodiment 65. The method according to any one of embodiments 34-64, wherein sequential treatments in which the oncolytic adenovirus is administered first, followed by systemic topotecan, enhance or increase cell infection and/or anticancer efficacy of the oncolytic adenovirus.

SEQUENCES

SEQ ID NO: 1
Complete PH20 cDNA sequence comprising the carboxy-terminal domain. (from ATG until stop codon,
both included, and with nt 1471-1527 underlined and bolded)
atgggagtgctaaaattcaagcacatctttttcagaagctttgttaaatcaagtggagtatcccagatagttttcaccttccttctgattccatg
ttgcttgactctgaatttcagagcacctcctgttattccaaatgtgcctttcctctgggcctggaatgccccaagtgaattttgtcttggaaaat
ttgatgagccactagatatgagcctcttctctttcataggaagcccccgaataaacgccaccgggcaaggtgttacaatattttatgttgataga
cttggctactatccttacatagattcaatcacaggagtaactgtgaatggaggaatcccccagaagatttccttacaagaccatctggacaaagc
taagaaagacattacattttatatgccagtagacaatttgggaatggctgttattgactgggaagaatggagacccacttgggcaagaaactgga
aacctaaagatgtttacaagaataggtctattgaattggttcagcaacaaaatgtacaacttagtctcacagaggccactgagaaagcaaaacaa
gaatttgaaaaggcagggaaggatttcctggtagagactataaaattgggaaaattacttcggccaaatcacttgtggggttattatctttttcc
ggattgttacaaccatcactataagaaacccggttacaatggaagttgcttcaatgtagaaataaaaagaaatgatgatctcagctggttgtgga
atgaaagcactgctctttacccatccatttatttgaacactcagcagtctcctgtagctgctacactctatgtgcgcaatcgagttcgggaagcc
atcagagtttccaaaatacctgatgcaaaaagtccacttccggtttttgcatatacccgcatagtttttactgatcaagttttgaaattcctttc
tcaagatgaacttgtgtatacatttggcgaaactgttgctctgggtgcttctggaattgtaatatggggaaccctcagtataatgcgaagtatga
aatcttgcttgctcctagacaattacatggagactatactgaatccttacataatcaacgtcacactagcagccaaaatgtgtagccaagtgctt
tgccaggagcaaggagtgtgtataaggaaaaactggaattcaagtgactatcttcacctcaacccagataattttgctattcaacttgagaaagg
tggaaagttcacagtacgtggaaaaccgacacttgaagacctggagcaattttctgaaaaattttattgcagctgttatagcaccttgagttgta
aggagaaagctgatgtaaaagacactgatgctgttgatgtgtgtattgctgatggtgtctgtatagatgcttttctaaaacctcccatggagaca
gaagaacctcaaattttctacaatgcttcaccctccacactatctgccacaatgttcattgttagtattttgtttcttatcatttcttctgtagc
gagtttgtaa
SEQ ID NO: 2
Sequence of the modified endogenous promoter of E1a + encoding E1a-Delta24 region in VCN-01
(comprising the four binding sites to E2F-1, the binding site to Sp1 and the encoding region of
E1a-Delta24)
aataagaggaagtgaaatctgaataattttgtgttactcatagcgcgtaatatttgtctagggccgcggggactttgaccgtttacgtggagact
cgcccaggtgtttttctcaggtgttttccgcgtacgtcggcggctcgtggctctttcgcggcaaaaaggatttggcgcgtaaaagtggttcgagt
acgtcggcggctcgtggctctttcgcggcaaaaaggatttggcgcgtaaaagtggttcgaagtacgtcgaccacaaaccccgcccagcgtcttgt
cattggcgtcgacgctgtacggggtcaaagttggcgttttattattatagtcagctgacgtgtagtgtatttatacccggtgagttcctcaagag
gccactcttgagtgccagcgagtagagttttctcctccgagccgctccgacaccgggactgaaaatgagacatattatctgccacggaggtgtta
ttaccgaagaaatggccgccagtcttttggaccagctgatcgaagaggtactggctgataatcttccacctcctagccattttgaaccacctacc
cttcacgaactgtatgatttagacgtgacggcccccgaagatcccaacgaggaggcggtttcgcagatttttcccgactctgtaatgttggcggt
gcaggaagggattgacttactcacttttccgccggcgcccggttctccggagccgcctcacctttcccggcagcccgagcagccggagcagagag
ccttgggtccggtttctatgccaaaccttgtaccggaggtgatcgatccacccagtgacgacgaggatgaagagggtgaggagtttgtgttagat
tatgtggagcaccccgggcacggttgcaggtcttgtcattatcaccggaggaatacgggggacccagatattatgtgttcgctttgctatatgag
gacctgtggcatgtttgtctacagtaagtgaaaattatgggcagtgggtgatagagtggtgggtttggtgtggtaattttttttttaatttttac
agttttgtggtttaaagaattttgtattgtgatttttttaaaaggtcctgtgtctgaacctgagcctgagcccgagccagaaccggagcctgcaa
gacctacccgccgtcctaaaatggcgcctgctatcctgagacgcccgacatcacctgtgtctagagaatgcaatagtagtacggatagctgtgac
tccggtccttctaacacacctcctgagatacacccggtggtcccgctgtgccccattaaaccagttgccgtgagagttggtgggcgtcgccaggc
tgtggaatgtatcgaggacttgcttaacgagcctgggcaacctttggacttgagctgtaaacgccccaggccataa
SEQ ID NO: 3
Complete sequence of VCN-01
catcatcaataatataccttattttggattgaagccaatatgataatgagggggtggagtttgtgacgtggcgcggggcgtgggaacggggcggg
tgacgtagtagtgtggcggaagtgtgatgttgcaagtgtggcggaacacatgtaagcgacggatgtggcaaaagtgacgtttttggtgtgcgccg
gtgtacacaggaagtgacaattttcgcgcggttttaggcggatgttgtagtaaatttgggcgtaaccgagtaagatttggccattttcgcgggaa
aactgaataagaggaagtgaaatctgaataattttgtgttactcatagcgcgtaatatttgtctagggccgcggggactttgaccgtttacgtgg
agactcgcccaggtgtttttctcaggtgttttccgcgtacgtcggcggctcgtggctctttcgcggcaaaaaggatttggcgcgtaaaagtggtt
cgagtacgtcggcggctcgtggctctttcgcggcaaaaaggatttggcgcgtaaaagtggttcgaagtacgtcgaccacaaaccccgcccagcgt
cttgtcattggcgtcgacgctgtacggggtcaaagttggcgttttattattatagtcagctgacgtgtagtgtatttatacccggtgagttcctc
aagaggccactcttgagtgccagcgagtagagttttctcctccgagccgctccgacaccgggactgaaaatgagacatattatctgccacggagg
tgttattaccgaagaaatggccgccagtcttttggaccagctgatcgaagaggtactggctgataatcttccacctcctagccattttgaaccac
ctacccttcacgaactgtatgatttagacgtgacggcccccgaagatcccaacgaggaggcggtttcgcagatttttcccgactctgtaatgttg
gcggtgcaggaagggattgacttactcacttttccgccggcgcccggttctccggagccgcctcacctttcccggcagcccgagcagccggagca
gagagccttgggtccggtttctatgccaaaccttgtaccggaggtgatcgatccacccagtgacgacgaggatgaagagggtgaggagtttgtgt
tagattatgtggagcaccccgggcacggttgcaggtcttgtcattatcaccggaggaatacgggggacccagatattatgtgttcgctttgctat
atgaggacctgtggcatgtttgtctacagtaagtgaaaattatgggcagtgggtgatagagtggtgggtttggtgtggtaattttttttttaatt
tttacagttttgtggtttaaagaattttgtattgtgatttttttaaaaggtcctgtgtctgaacctgagcctgagcccgagccagaaccggagcc
tgcaagacctacccgccgtcctaaaatggcgcctgctatcctgagacgcccgacatcacctgtgtctagagaatgcaatagtagtacggatagct
gtgactccggtccttctaacacacctcctgagatacacccggtggtcccgctgtgccccattaaaccagttgccgtgagagttggtgggcgtcgc
caggctgtggaatgtatcgaggacttgcttaacgagcctgggcaacctttggacttgagctgtaaacgccccaggccataaggtgtaaacctgtg
attgcgtgtgtggttaacgcctttgtttgctgaatgagttgatgtaagtttaataaagggtgagataatgtttaacttgcatggcgtgttaaatg
gggggggcttaaagggtatataatgcgccgtgggctaatcttggttacatctgacctcatggaggcttgggagtgtttggaagatttttctgctg
tgcgtaacttgctggaacagagctctaacagtacctcttggttttggaggtttctgtggggctcatcccaggcaaagttagtctgcagaattaag
gaggattacaagtgggaatttgaagagcttttgaaatcctgtggtgagctgtttgattctttgaatctgggtcaccaggcgcttttccaagagaa
ggtcatcaagactttggatttttccacaccggggcgcgctgcggctgctgttgcttttttgagttttataaaggataaatggagcgaagaaaccc
atctgagcggggggtacctgctggattttctggccatgcatctgtggagagcggttgtgagacacaagaatcgcctgctactgttgtcttccgtc
cgcccggcgataataccgacggaggagcagcagcagcagcaggaggaagccaggcggcggcggcaggagcagagcccatggaacccgagagccgg
cctggaccctcgggaatgaatgttgtacaggtggctgaactgtatccagaactgagacgcattttgacaattacagaggatgggcaggggctaaa
gggggtaaagagggagcggggggcttgtgaggctacagaggaggctaggaatctagcttttagcttaatgaccagacaccgtcctgagtgtatta
cttttcaacagatcaaggataattgcgctaatgagcttgatctgctggcgcagaagtattccatagagcagctgaccacttactggctgcagcca
ggggatgattttgaggaggctattagggtatatgcaaaggtggcacttaggccagattgcaagtacaagatcagcaaacttgtaaatatcaggaa
ttgttgctacatttctgggaacggggccgaggtggagatagatacggaggatagggggcctttagatgtagcatgataaatatgtggccgggggt
gcttggcatggacggggtggttattatgaatgtaaggtttactggccccaattttagcggtacggttttcctggccaataccaaccttatcctac
acggtgtaagcttctatgggtttaacaatacctgtgtggaagcctggaccgatgtaagggttcggggctgtgccttttactgctgctggaagggg
gtggtgtgtcgccccaaaagcagggcttcaattaagaaatgcctctttgaaaggtgtaccttgggtatcctgtctgagggtaactccagggtgcg
ccacaatgtggcctccgactgtggttgcttcatgctagtgaaaagcgtggctgtgattaagcataacatggtatgtggcaactgcgaggacaggg
cctctcagatgctgacctgctcggacggcaactgtcacctgctgaagaccattcacgtagccagccactctcgcaaggcctggccagtgtttgag
cataacatactgacccgctgttccttgcatttgggtaacaggaggggggtgttcctaccttaccaatgcaatttgagtcacactaagatattgct
tgagcccgagagcatgtccaaggtgaacctgaacggggtgtttgacatgaccatgaagatctggaaggtgctgaggtacgatgagacccgcacca
ggtgcagaccctgcgagtgtggcggtaaacatattaggaaccagcctgtgatgctggatgtgaccgaggagctgaggcccgatcacttggtgctg
gcctgcacccgcgctgagtttggctctagcgatgaagatacagattgaggtactgaaatgtgtgggcgtggcttaaggggggaaagaatatataa
ggtgggggtcttatgtagttttgtatctgttttgcagcagccgccgccgccatgagcaccaactcgtttgatggaagcattgtgagctcatattt
gacaacgcgcatgcccccatgggccggggtgcgtcagaatgtgatgggctccagcattgatggtcgccccgtcctgcccgcaaactctactacct
tgacctacgagaccgtgtctggaacgccgttggagactgcagcctccgccgccgcttcagccgctgcagccaccgcccgcgggattgtgactgac
tttgctttcctgagcccgcttgcaagcagtgcagcttcccgttcatccgcccgcgatgacaagttgacggctcttttggcacaattggattcttt
gacccgggaacttaatgtcgtttctcagcagctgttggatctgcgccagcaggtttctgccctgaaggcttcctcccctcccaatgcggtttaaa
acataaataaaaaaccagactctgtttggatttggatcaagcaagtgtcttgctgtctttatttaggggttttgcgcgcgcggtaggcccgggac
cagcggtctcggtcgttgagggtcctgtgtattttttccaggacgtggtaaaggtgactctggatgttcagatacatgggcataagcccgtctct
ggggtggaggtagcaccactgcagagcttcatgctgcggggtggtgttgtagatgatccagtcgtagcaggagcgctgggcgtggtgcctaaaaa
tgtctttcagtagcaagctgattgccaggggcaggcccttggtgtaagtgtttacaaagcggttaagctgggatgggtgcatacgtggggatatg
agatgcatcttggactgtatttttaggttggctatgttcccagccatatccctccggggattcatgttgtgcagaaccaccagcacagtgtatcc
ggtgcacttgggaaatttgtcatgtagcttagaaggaaatgcgtggaagaacttggagacgcccttgtgacctccaagattttccatgcattcgt
ccataatgatggcaatgggcccacgggcggcggcctgggcgaagatatttctgggatcactaacgtcatagttgtgttccaggatgagatcgtca
taggccatttttacaaagcgcgggcggagggtgccagactgcggtataatggttccatccggcccaggggcgtagttaccctcacagatttgcat
ttcccacgctttgagttcagatggggggatcatgtctacctgcggggcgatgaagaaaacggtttccggggtaggggagatcagctgggaagaaa
gcaggttcctgagcagctgcgacttaccgcagccggtgggcccgtaaatcacacctattaccggctgcaactggtagttaagagagctgcagctg
ccgtcatccctgagcaggggggccacttcgttaagcatgtccctgactcgcatgttttccctgaccaaatccgccagaaggcgctcgccgcccag
cgatagcagttcttgcaaggaagcaaagtttttcaacggtttgagaccgtccgccgtaggcatgcttttgagcgtttgaccaagcagttccaggc
ggtcccacagctcggtcacctgctctacggcatctcgatccagcatatctcctcgtttcgcgggttggggcggctttcgctgtacggcagtagtc
ggtgctcgtccagacgggccagggtcatgtctttccacgggcgcagggtcctcgtcagcgtagtctgggtcacggtgaaggggtgcgctccgggc
tgcgcgctggccagggtgcgcttgaggctggtcctgctggtgctgaagcgctgccggtcttcgccctgcgcgtcggccaggtagcatttgaccat
ggtgtcatagtccagcccctccgcggcgtggcccttggcgcgcagcttgcccttggaggaggcgccgcacgaggggcagtgcagacttttgaggg
cgtagagcttgggcgcgagaaataccgattccggggagtaggcatccgcgccgcaggccccgcagacggtctcgcattccacgagccaggtgagc
tctggccgttcggggtcaaaaaccaggtttcccccatgctttttgatgcgtttcttacctctggtttccatgagccggtgtccacgctcggtgac
gaaaaggctgtccgtgtccccgtatacagacttgagaggcctgtcctcgagcggtgttccgcggtcctcctcgtatagaaactcggaccactctg
agacaaaggctcgcgtccaggccagcacgaaggaggctaagtgggaggggtagcggtcgttgtccactagggggtccactcgctccagggtgtga
agacacatgtcgccctcttcggcatcaaggaaggtgattggtttgtaggtgtaggccacgtgaccgggtgttcctgaaggggggctataaaaggg
ggtgggggcgcgttcgtcctcactctcttccgcatcgctgtctgcgagggccagctgttggggtgagtactccctctgaaaagcgggcatgactt
ctgcgctaagattgtcagtttccaaaaacgaggaggatttgatattcacctggcccgcggtgatgcctttgagggtggccgcatccatctggtca
gaaaagacaatctttttgttgtcaagcttggtggcaaacgacccgtagagggcgttggacagcaacttggcgatggagcgcagggtttggttttt
gtcgcgatcggcgcgctccttggccgcgatgtttagctgcacgtattcgcgcgcaacgcaccgccattcgggaaagacggtggtgcgctcgtcgg
gcaccaggtgcacgcgccaaccgcggttgtgcagggtgacaaggtcaacgctggtggctacctctccgcgtaggcgctcgttggtccagcagagg
cggccgcccttgcgcgagcagaatggcggtagggggtctagctgcgtctcgtccggggggtctgcgtccacggtaaagaccccgggcagcaggcg
cgcgtcgaagtagtctatcttgcatccttgcaagtctagcgcctgctgccatgcgcgggcggcaagcgcgcgctcgtatgggttgagtgggggac
cccatggcatggggtgggtgagcgcggaggcgtacatgccgcaaatgtcgtaaacgtagaggggctctctgagtattccaagatatgtagggtag
catcttccaccgcggatgctggcgcgcacgtaatcgtatagttcgtgcgagggagcgaggaggtcgggaccgaggttgctacgggcgggctgctc
tgctcggaagactatctgcctgaagatggcatgtgagttggatgatatggttggacgctggaagacgttgaagctggcgtctgtgagacctaccg
cgtcacgcacgaaggaggcgtaggagtcgcgcagcttgttgaccagctcggcggtgacctgcacgtctagggcgcagtagtccagggtttccttg
atgatgtcatacttatcctgtcccttttttttccacagctcgcggttgaggacaaactcttcgcggtctttccagtactcttggatcggaaaccc
gtcggcctccgaacggtaagagcctagcatgtagaactggttgacggcctggtaggcgcagcatcccttttctacgggtagcgcgtatgcctgcg
cggccttccggagcgaggtgtgggtgagcgcaaaggtgtccctgaccatgactttgaggtactggtatttgaagtcagtgtcgtcgcatccgccc
tgctcccagagcaaaaagtccgtgcgctttttggaacgcggatttggcagggcgaaggtgacatcgttgaagagtatctttcccgcgcgaggcat
aaagttgcgtgtgatgcggaagggtcccggcacctcggaacggttgttaattacctgggcggcgagcacgatctcgtcaaagccgttgatgttgt
ggcccacaatgtaaagttccaagaagcgcgggatgcccttgatggaaggcaattttttaagttcctcgtaggtgagctcttcaggggagctgagc
ccgtgctctgaaagggcccagtctgcaagatgagggttggaagcgacgaatgagctccacaggtcacgggccattagcatttgcaggtggtcgcg
aaaggtcctaaactggcgacctatggccattttttctggggtgatgcagtagaaggtaagcgggtcttgttcccagcggtcccatccaaggttcg
cggctaggtctcgcgcggcagtcactagaggctcatctccgccgaacttcatgaccagcatgaagggcacgagctgcttcccaaaggcccccatc
caagtataggtctctacatcgtaggtgacaaagagacgctcggtgcgaggatgcgagccgatcgggaagaactggatctcccgccaccaattgga
ggagtggctattgatgtggtgaaagtagaagtccctgcgacgggccgaacactcgtgctggcttttgtaaaaacgtgcgcagtactggcagcggt
gcacgggctgtacatcctgcacgaggttgacctgacgaccgcgcacaaggaagcagagtgggaatttgagcccctcgcctgggggtttggctggt
ggtcttctacttcggctgcttgtccttgaccgtctggctgctcgaggggagttacggtggatcggaccaccacgccgcgcgagcccaaagtccag
atgtccgcgcgcggcggtcggagcttgatgacaacatcgcgcagatgggagctgtccatggtctggagctcccgcggcgtcaggtcaggcgggag
ctcctgcaggtttacctcgcatagacgggtcagggcgcgggctagatccaggtgatacctaatttccaggggctggttggtggcggcgtcgatgg
cttgcaagaggccgcatccccgcggcgcgactacggtaccgcgcggcgggcggtgggccgcgggggtgtccttggatgatgcatctaaaagcggt
gacgcgggcgagcccccggaggtagggggggctccggacccgccgggagagggggcaggggcacgtcggcgccgcgcgcgggcaggagctggtgc
tgcgcgcgtaggttgctggcgaacgcgacgacgcggcggttgatctcctgaatctggcgcctctgcgtgaagacgacgggcccggtgagcttgaa
cctgaaagagagttcgacagaatcaatttcggtgtcgttgacggcggcctggcgcaaaatctcctgcacgtctcctgagttgtcttgataggcga
tctcggccatgaactgctcgatctcttcctcctggagatctccgcgtccggctcgctccacggtggcggcgaggtcgttggaaatgcgggccatg
agctgcgagaaggcgttgaggcctccctcgttccagacgcggctgtagaccacgcccccttcggcatcgcgggcgcgcatgaccacctgcgcgag
attgagctccacgtgccgggcgaagacggcgtagtttcgcaggcgctgaaagaggtagttgagggtggtggcggtgtgttctgccacgaagaagt
acataacccagcgtcgcaacgtggattcgttgatatcccccaaggcctcaaggcgctccatggcctcgtagaagtccacggcgaagttgaaaaac
tgggagttgcgcgccgacacggttaactcctcctccagaagacggatgagctcggcgacagtgtcgcgcacctcgcgctcaaaggctacaggggc
ctcttcttcttcttcaatctcctcttccataagggcctccccttcttcttcttctggcggcggtgggggaggggggacacggggcgacgacggcg
caccgggaggcggtcgacaaagcgctcgatcatctccccgcggcgacggcgcatggtctcggtgacggcgcggccgttctcgcgggggcgcagtt
ggaagacgccgcccgtcatgtcccggttatgggttggcggggggctgccatgcggcagggatacggcgctaacgatgcatctcaacaattgttgt
gtaggtactccgccgccgagggacctgagcgagtccgcatcgaccggatcggaaaacctctcgagaaaggcgtctaaccagtcacagtcgcaagg
taggctgagcaccgtggcgggcggcagcgggcggcggtcggggttgtttctggcggaggtgctgctgatgatgtaattaaagtaggcggtcttga
gacggcggatggtcgacagaagcaccatgtccttgggtccggcctgctgaatgcgcaggcggtcggccatgccccaggcttcgttttgacatcgg
cgcaggtctttgtagtagtcttgcatgagcctttctaccggcacttcttcttctccttcctcttgtcctgcatctcttgcatctatcgctgcggc
ggcggcggagtttggccgtaggtggcgccctcttcctcccatgcgtgtgaccccgaagcccctcatcggctgaagcagggctaggtcggcgacaa
cgcgctcggctaatatggcctgctgcacctgcgtgagggtagactggaagtcatccatgtccacaaagcggtggtatgcgcccgtgttgatggtg
taagtgcagttggccataacggaccagttaacggtctggtgacccggctgcgagagctcggtgtacctgagacgcgagtaagccctcgagtcaaa
tacgtagtcgttgcaagtccgcaccaggtactggtatcccaccaaaaagtgcggcggcggctggcggtagaggggccagcgtagggtggccgggg
ctccgggggcgagatcttccaacataaggcgatgatatccgtagatgtacctggacatccaggtgatgccggcggcggtggtggaggcgcgcgga
aagtcgcggacgcggttccagatgttgcgcagcggcaaaaagtgctccatggtcgggacgctctggccggtcaggcgcgcgcaatcgttgacgct
ctagaccgtgcaaaaggagagcctgtaagcgggcactcttccgtggtctggtggataaattcgcaagggtatcatggcggacgaccggggttcga
gccccgtatccggccgtccgccgtgatccatgcggttaccgcccgcgtgtcgaacccaggtgtgcgacgtcagacaacgggggagtgctcctttt
ggcttccttccaggcgcggcggctgctgcgctagcttttttggccactggccgcgcgcagcgtaagcggttaggctggaaagcgaaagcattaag
tggctcgctccctgtagccggagggttattttccaagggttgagtcgcgggacccccggttcgagtctcggaccggccggactgcggcgaacggg
ggtttgcctccccgtcatgcaagaccccgcttgcaaattcctccggaaacagggacgagccccttttttgcttttcccagatgcatccggtgctg
cggcagatgcgcccccctcctcagcagoggcaagagcaagagcagcggcagacatgcagggcaccctcccctcctcctaccgcgtcaggaggggc
gacatccgcggttgacgcggcagcagatggtgattacgaacccccgcggcgccgggcccggcactacctggacttggaggagggcgagggcctgg
cgcggctaggagcgccctctcctgagcggcacccaagggtgcagctgaagcgtgatacgcgtgaggcgtacgtgccgcggcagaacctgtttcgc
gaccgcgagggagaggagcccgaggagatgcgggatcgaaagttccacgcagggcgcgagctgcggcatggcctgaatcgcgagcggttgctgcg
cgaggaggactttgagcccgacgcgcgaaccgggattagtcccgcgcgcgcacacgtggcggccgccgacctggtaaccgcatacgagcagacgg
tgaaccaggagattaactttcaaaaaagctttaacaaccacgtgcgtacgcttgtggcgcgcgaggaggtggctataggactgatgcatctgtgg
gactttgtaagcgcgctggagcaaaacccaaatagcaagccgctcatggcgcagctgttccttatagtgcagcacagcagggacaacgaggcatt
cagggatgcgctgctaaacatagtagagcccgagggccgctggctgctcgatttgataaacatcctgcagagcatagtggtgcaggagcgcagct
tgagcctggctgacaaggtggccgccatcaactattccatgcttagcctgggcaagttttacgcccgcaagatataccataccccttacgttccc
atagacaaggaggtaaagatcgaggggttctacatgcgcatggcgctgaaggtgcttaccttgagcgacgacctgggcgtttatcgcaacgagcg
catccacaaggccgtgagcgtgagccggcggcgcgagctcagcgaccgcgagctgatgcacagcctgcaaagggccctggctggcacgggcagcg
gcgatagagaggccgagtcctactttgacgcgggcgctgacctgcgctgggccccaagccgacgcgccctggaggcagctggggccggacctggg
ctggcggtggcacccgcgcgcgctggcaacgtcggcggcgtggaggaatatgacgaggacgatgagtacgagccagaggacggcgagtactaagc
ggtgatgtttctgatcagatgatgcaagacgcaacggacccggcggtgcgggcggcgctgcagagccagccgtccggccttaactccacggacga
ctggcgccaggtcatggaccgcatcatgtcgctgactgcgcgcaatcctgacgcgttccggcagcagccgcaggccaaccggctctccgcaattc
tggaagcggtggtcccggcgcgcgcaaaccccacgcacgagaaggtgctggcgatcgtaaacgcgctggccgaaaacagggccatccggcccgac
gaggccggcctggtctacgacgcgctgcttcagcgcgtggctcgttacaacagcggcaacgtgcagaccaacctggaccggctggtgggggatgt
gcgcgaggccgtggcgcagcgtgagcgcgcgcagcagcagggcaacctgggctccatggttgcactaaacgccttcctgagtacacagcccgcca
acgtgccgcggggacaggaggactacaccaactttgtgagcgcactgcggctaatggtgactgagacaccgcaaagtgaggtgtaccagtctggg
ccagactattttttccagaccagtagacaaggcctgcagaccgtaaacctgagccaggctttcaaaaacttgcaggggctgtggggggtgcgggc
tcccacaggcgaccgcgcgaccgtgtctagcttgctgacgcccaactcgcgcctgttgctgctgctaatagcgcccttcacggacagtggcagcg
tgtcccgggacacatacctaggtcacttgctgacactgtaccgcgaggccataggtcaggcgcatgtggacgagcatactttccaggagattaca
agtgtcagccgcgcgctggggcaggaggacacgggcagcctggaggcaaccctaaactacctgctgaccaaccggcggcagaagatcccctcgtt
gcacagtttaaacagcgaggaggagcgcattttgcgctacgtgcagcagagcgtgagccttaacctgatgcgcgacggggtaacgcccagcgtgg
cgctggacatgaccgcgcgcaacatggaaccgggcatgtatgcctcaaaccggccgtttatcaaccgcctaatggactacttgcatcgcgcggcc
gccgtgaaccccgagtatttcaccaatgccatcttgaacccgcactggctaccgccccctggtttctacaccgggggattcgaggtgcccgaggg
taacgatggattcctctgggacgacatagacgacagcgtgttttccccgcaaccgcagaccctgctagagttgcaacagcgcgagcaggcagagg
cggcgctgcgaaaggaaagcttccgcaggccaagcagcttgtccgatctaggcgctgcggccccgcggtcagatgctagtagcccatttccaagc
ttgatagggtctcttaccagcactcgcaccacccgcccgcgcctgctgggcgaggaggagtacctaaacaactcgctgctgcagccgcagcgcga
aaaaaacctgcctccggcatttcccaacaacgggatagagagcctagtggacaagatgagtagatggaagacgtacgcgcaggagcacagggacg
tgccaggcccgcgcccgcccacccgtcgtcaaaggcacgaccgtcagcggggtctggtgtgggaggacgatgactcggcagacgacagcagcgtc
ctggatttgggagggagtggcaacccgtttgcgcaccttcgccccaggctggggagaatgttttaaaaaaaaaaaaagcatgatgcaaaataaaa
aactcaccaaggccatggcaccgagcgttggttttcttgtattccccttagtatgcggcgcgcggcgatgtatgaggaaggtcctcctccctcct
acgagagtgtggtgagcgcggcgccagtggcggcggcgctgggttctcccttcgatgctcccctggacccgccgtttgtgcctccgcggtacctg
cggcctaccggggggagaaacagcatccgttactctgagttggcacccctattcgacaccacccgtgtgtacctggtggacaacaagtcaacgga
tgtggcatccctgaactaccagaacgaccacagcaactttctgaccacggtcattcaaaacaatgactacagcccgggggaggcaagcacacaga
ccatcaatcttgacgaccggtcgcactggggcggcgacctgaaaaccatcctgcataccaacatgccaaatgtgaacgagttcatgtttaccaat
aagtttaaggcgcgggtgatggtgtcgcgcttgcctactaaggacaatcaggtggagctgaaatacgagtgggtggagttcacgctgcccgaggg
caactactccgagaccatgaccatagaccttatgaacaacgcgatcgtggagcactacttgaaagtgggcagacagaacggggttctggaaagcg
acatcggggtaaagtttgacacccgcaacttcagactggggtttgaccccgtcactggtcttgtcatgcctggggtatatacaaacgaagccttc
catccagacatcattttgctgccaggatgcggggtggacttcacccacagccgcctgagcaacttgttgggcatccgcaagcggcaacccttcca
ggagggctttaggatcacctacgatgatctggagggtggtaacattcccgcactgttggatgtggacgcctaccaggcgagcttgaaagatgaca
ccgaacaggggggggtggcgcaggcggcagcaacagcagtggcagcggcgcggaagagaactccaacgcggcagccgcggcaatgcagccggtgg
aggacatgaacgatcatgccattcgcggcgacacctttgccacacgggctgaggagaagcgcgctgaggccgaagcagcggccgaagctgccgcc
cccgctgcgcaacccgaggtcgagaagcctcagaagaaaccggtgatcaaacccctgacagaggacagcaagaaacgcagttacaacctaataag
caatgacagcaccttcacccagtaccgcagctggtaccttgcatacaactacggcgaccctcagaccggaatccgctcatggaccctgctttgca
ctcctgacgtaacctgcggctcggagcaggtctactggtcgttgccagacatgatgcaagaccccgtgaccttccgctccacgcgccagatcagc
aactttccggtggtgggcgccgagctgttgcccgtgcactccaagagcttctacaacgaccaggccgtctactcccaactcatccgccagtttac
ctctctgacccacgtgttcaatcgctttcccgagaaccagattttggcgcgcccgccagcccccaccatcaccaccgtcagtgaaaacgttcctg
ctctcacagatcacgggacgctaccgctgcgcaacagcatcggaggagtccagcgagtgaccattactgacgccagacgccgcacctgcccctac
gtttacaaggccctgggcatagtctcgccgcgcgtcctatcgagccgcactttttgagcaagcatgtccatccttatatcgcccagcaataacac
aggctggggcctgcgcttcccaagcaagatgtttggcggggccaagaagcgctccgaccaacacccagtgcgcgtgcgcgggcactaccgcgcgc
cctggggcgcgcacaaacgcggccgcactgggcgcaccaccgtcgatgacgccatcgacgcggtggtggaggaggcgcgcaactacacgcccacg
ccgccaccagtgtccacagtggacgcggccattcagaccgtggtgcgcggagcccggcgctatgctaaaatgaagagacggcggaggcgcgtagc
acgtcgccaccgccgccgacccggcactgccgcccaacgcgcggcggcggccctgcttaaccgcgcacgtcgcaccggccgacgggggccatgcg
ggccgctcgaaggctggccgcgggtattgtcactgtgccccccaggtccaggcgacgagcggccgccgcagcagccgcggccattagtgctatga
ctcagggtcgcaggggcaacgtgtattgggtgcgcgactcggttagcggcctgcgcgtgcccgtgcgcacccgccccccgcgcaactagattgca
agaaaaaactacttagactcgtactgttgtatgtatccagcggcggcggcgcgcaacgaagctatgtccaagcgcaaaatcaaagaagagatgct
ccaggtcatcgcgccggagatctatggccccccgaagaaggaagagcaggattacaagccccgaaagctaaagcgggtcaaaaagaaaaagaaag
atgatgatgatgaacttgacgacgaggtggaactgctgcacgctaccgcgcccaggcgacgggtacagtggaaaggtcgacgcgtaaaacgtgtt
ttgcgacccggcaccaccgtagtctttacgcccggtgagcgctccacccgcacctacaagcgcgtgtatgatgaggtgtacggcgacgaggacct
gcttgagcaggccaacgagcgcctcggggagtttgcctacggaaagcggcataaggacatgctggcgttgccgctggacgagggcaacccaacac
ctagcctaaagcccgtaacactgcagcaggtgctgcccgcgcttgcaccgtccgaagaaaagcgcggcctaaagcgcgagtctggtgacttggca
cccaccgtgcagctgatggtacccaagcgccagcgactggaagatgtcttggaaaaaatgaccgtggaacctgggctggagcccgaggtccgcgt
gcggccaatcaagcaggtggcgccgggactgggcgtgcagaccgtggacgttcagatacccactaccagtagcaccagtattgccaccgccacag
agggcatggagacacaaacgtccccggttgcctcagcggtggcggatgccgcggtgcaggcggtcgctgcggccgcgtccaagacctctacggag
gtgcaaacggacccgtggatgtttcgcgtttcagccccccggcgcccgcgccgttcgaggaagtacggcgccgccagcgcgctactgcccgaata
tgccctacatccttccattgcgcctacccccggctatcgtggctacacctaccgccccagaagacgagcaactacccgacgccgaaccaccactg
gaacccgccgccgccgtcgccgtcgccagcccgtgctggccccgatttccgtgcgcagggtggctcgcgaaggaggcaggaccctggtgctgcca
acagcgcgctaccaccccagcatcgtttaaaagccggtctttgtggttcttgcagatatggccctcacctgccgcctccgtttcccggtgccggg
attccgaggaagaatgcaccgtaggaggggcatggccggccacggcctgacgggcggcatgcgtcgtgcgcaccaccggcggcggcgcgcgtcgc
accgtcgcatgcgcggcggtatcctgcccctccttattccactgatcgccgcggcgattggcgccgtgcccggaattgcatccgtggccttgcag
gcgcagagacactgattaaaaacaagttgcatgtggaaaaatcaaaataaaaagtctggactctcacgctcgcttggtcctgtaactattttgta
gaatggaagacatcaactttgcgtctctggccccgcgacacggctcgcgcccgttcatgggaaactggcaagatatcggcaccagcaatatgagc
ggtggcgccttcagctggggctcgctgtggagcggcattaaaaatttcggttccaccgttaagaactatggcagcaaggcctggaacagcagcac
aggccagatgctgagggataagttgaaagagcaaaatttccaacaaaaggtggtagatggcctggcctctggcattagcggggtggtggacctgg
ccaaccaggcagtgcaaaataagattaacagtaagcttgatccccgccctcccgtagaggagcctccaccggccgtggagacagtgtctccagag
gggcgtggcgaaaagcgtccgcgccccgacagggaagaaactctggtgacgcaaatagacgagcctccctcgtacgaggaggcactaaagcaagg
cctgcccaccacccgtcccatcgcgcccatggctaccggagtgctgggccagcacacacccgtaacgctggacctgcctccccccgccgacaccc
agcagaaacctgtgctgccaggcccgaccgccgttgttgtaacccgtcctagccgcgcgtccctgcgccgcgccgccagcggtccgcgatcgttg
cggcccgtagccagtggcaactggcaaagcacactgaacagcatcgtgggtctgggggtgcaatccctgaagcgccgacgatgcttctgatagct
aacgtgtcgtatgtgtgtcatgtatgcgtccatgtcgccgccagaggagctgctgagccgccgcgcgcccgctttccaagatggctaccccttcg
atgatgccgcagtggtcttacatgcacatctcgggccaggacgcctcggagtacctgagccccgggctggtgcagtttgcccgcgccaccgagac
gtacttcagcctgaataacaagtttagaaaccccacggtggcgcctacgcacgacgtgaccacagaccggtcccagcgtttgacgctgcggttca
tccctgtggaccgtgaggatactgcgtactcgtacaaggcgcggttcaccctagctgtgggtgataaccgtgtgctggacatggcttccacgtac
tttgacatccgcggcgtgctggacaggggccctacttttaagccctactctggcactgcctacaacgccctggctcccaagggtgccccaaatcc
ttgcgaatgggatgaagctgctactgctcttgaaataaacctagaagaagaggacgatgacaacgaagacgaagtagacgagcaagctgagcagc
aaaaaactcacgtatttgggcaggcgccttattctggtataaatattacaaaggagggtattcaaataggtgtcgaaggtcaaacacctaaatat
gccgataaaacatttcaacctgaacctcaaataggagaatctcagtggtacgaaacagaaattaatcatgcagctgggagagtcctaaaaaagac
taccccaatgaaaccatgttacggttcatatgcaaaacccacaaatgaaaatggagggcaaggcattcttgtaaagcaacaaaatggaaagctag
aaagtcaagtggaaatgcaatttttctcaactactgaggcagccgcaggcaatggtgataacttgactcctaaagtggtattgtacagtgaagat
gtagatatagaaaccccagacactcatatttottacatgcccactattaaggaaggtaactcacgagaactaatgggccaacaatctatgcccaa
caggcctaattacattgcttttagggacaattttattggtctaatgtattacaacagcacgggtaatatgggtgttctggcgggccaagcatcgc
agttgaatgctgttgtagatttgcaagacagaaacacagagctttcataccagcttttgcttgattccattggtgatagaaccaggtacttttct
atgtggaatcaggctgttgacagctatgatccagatgttagaattattgaaaatcatggaactgaagatgaacttccaaattactgctttccact
gggaggtgtgattaatacagagactcttaccaaggtaaaacctaaaacaggtcaggaaaatggatgggaaaaagatgctacagaattttcagata
aaaatgaaataagagttggaaataattttgccatggaaatcaatctaaatgccaacctgtggagaaatttcctgtactccaacatagcgctgtat
ttgcccgacaagctaaagtacagtccttccaacgtaaaaatttctgataacccaaacacctacgactacatgaacaagcgagtggtggctcccgg
gctagtggactgctacattaaccttggagcacgctggtcccttgactatatggacaacgtcaacccatttaaccaccaccgcaatgctggcctgc
gctaccgctcaatgttgctgggcaatggtcgctatgtgcccttccacatccaggtgcctcagaagttctttgccattaaaaacctccttctcctg
ccgggctcatacacctacgagtggaacttcaggaaggatgttaacatggttctgcagagctccctaggaaatgacctaagggttgacggagccag
cattaagtttgatagcatttgcctttacgccaccttcttccccatggcccacaacaccgcctccacgcttgaggccatgcttagaaacgacacca
acgaccagtcctttaacgactatctctccgccgccaacatgctctaccctatacccgccaacgctaccaacgtgcccatatccatcccctcccgc
aactgggcggctttccgcggctgggccttcacgcgccttaagactaaggaaaccccatcactgggctcgggctacgacccttattacacctactc
tggctctataccctacctagatggaaccttttacctcaaccacacctttaagaaggtggccattacctttgactcttctgtcagctggcctggca
atgaccgcctgcttacccccaacgagtttgaaattaagcgctcagttgacggggagggttacaacgttgcccagtgtaacatgaccaaagactgg
ttcctggtacaaatgctagctaactataacattggctaccagggcttctatatcccagagagctacaaggaccgcatgtactccttctttagaaa
cttccagcccatgagccgtcaggtggtggatgatactaaatacaaggactaccaacaggtgggcatcctacaccaacacaacaactctggatttg
ttggctaccttgcccccaccatgcgcgaaggacaggcctaccctgctaacttcccctatccgcttataggcaagaccgcagttgacagcattacc
cagaaaaagtttctttgcgatcgcaccctttggcgcatcccattctccagtaactttatgtccatgggcgcactcacagacctgggccaaaacct
tctctacgccaactccgcccacgcgctagacatgacttttgaggtggatcccatggacgagcccacccttctttatgttttgtttgaagtctttg
acgtggtccgtgtgcaccagccgcaccgcggcgtcatcgaaaccgtgtacctgcgcacgcccttctcggccggcaacgccacaacataaagaagc
aagcaacatcaacaacagctgccgccatgggctccagtgagcaggaactgaaagccattgtcaaagatcttggttgtgggccatattttttgggc
acctatgacaagcgctttccaggctttgtttctccacacaagctcgcctgcgccatagtcaatacggccggtcgcgagactgggggcgtacactg
gatggcctttgcctggaacccgcactcaaaaacatgctacctctttgagccctttggcttttctgaccagcgactcaagcaggtttaccagtttg
agtacgagtcactcctgcgccgtagcgccattgcttcttcccccgaccgctgtataacgctggaaaagtccacccaaagcgtacaggggcccaac
tcggccgcctgtggactattctgctgcatgtttctccacgcctttgccaactggccccaaactcccatggatcacaaccccaccatgaaccttat
taccggggtacccaactccatgctcaacagtccccaggtacagcccaccctgcgtcgcaaccaggaacagctctacagcttcctggagcgccact
cgccctacttccgcagccacagtgcgcagattaggagcgccacttctttttgtcacttgaaaaacatgtaaaaataatgtactagagacactttc
aataaaggcaaatgcttttatttgtacactctcgggtgattatttacccccacccttgccgtctgcgccgtttaaaaatcaaaggggttctgccg
cgcatcgctatgcgccactggcagggacacgttgcgatactggtgtttagtgctccacttaaactcaggcacaaccatccgcggcagctcggtga
agttttcactccacaggctgcgcaccatcaccaacgcgtttagcaggtcgggcgccgatatcttgaagtcgcagttggggcctccgccctgcgcg
cgcgagttgcgatacacagggttgcagcactggaacactatcagcgccgggtggtgcacgctggccagcacgctcttgtcggagatcagatccgc
gtccaggtcctccgcgttgctcagggcgaacggagtcaactttggtagctgccttcccaaaaagggcgcgtgcccaggctttgagttgcactcgc
accgtagtggcatcaaaaggtgaccgtgcccggtctgggcgttaggatacagcgcctgcataaaagccttgatctgcttaaaagccacctgagcc
tttgcgccttcagagaagaacatgccgcaagacttgccggaaaactgattggccggacaggccgcgtcgtgcacgcagcaccttgcgtcggtgtt
ggagatctgcaccacatttcggccccaccggttcttcacgatcttggccttgctagactgctccttcagcgcgcgctgcccgttttcgctcgtca
catccatttcaatcacgtgctccttatttatcataatgcttccgtgtagacacttaagctcgccttcgatctcagcgcagcggtgcagccacaac
gcgcagcccgtgggctcgtgatgcttgtaggtcacctctgcaaacgactgcaggtacgcctgcaggaatcgccccatcatcgtcacaaaggtctt
gttgctggtgaaggtcagctgcaacccgcggtgctcctcgttcagccaggtcttgcatacggccgccagagcttccacttggtcaggcagtagtt
tgaagttcgcctttagatcgttatccacgtggtacttgtccatcagcgcgcgcgcagcctccatgcccttctcccacgcagacacgatcggcaca
ctcagcgggttcatcaccgtaatttcactttccgcttcgctgggctcttcctcttcctcttgcgtccgcataccacgcgccactgggtcgtcttc
attcagccgccgcactgtgcgcttacctcctttgccatgcttgattagcaccggtgggttgctgaaacccaccatttgtagcgccacatcttctc
tttcttcctcgctgtccacgattacctctggtgatggcgggcgctcgggcttgggagaagggcgcttctttttcttcttgggcgcaatggccaaa
tccgccgccgaggtcgatggccgcgggctgggtgtgcgcggcaccagcgcgtcttgtgatgagtcttcctcgtcctcggactcgatacgccgcct
catccgcttttttgggggcgcccggggaggcggcggcgacggggacggggacgacacgtcctccatggttgggggacgtcgcgccgcaccgcgtc
cgcgctcgggggtggtttcgcgctgctcctcttcccgactggccatttccttctcctataggcagaaaaagatcatggagtcagtcgagaagaag
gacagcctaaccgccccctctgagttcgccaccaccgcctccaccgatgccgccaacgcgcctaccaccttccccgtcgaggcacccccgcttga
ggaggaggaagtgattatcgagcaggacccaggttttgtaagcgaagacgacgaggaccgctcagtaccaacagaggataaaaagcaagaccagg
acaacgcagaggcaaacgaggaacaagtcgggggggggacgaaaggcatggcgactacctagatgtgggagacgacgtgctgttgaagcatctgc
agcgccagtgcgccattatctgcgacgcgttgcaagagcgcagcgatgtgcccctcgccatagcggatgtcagccttgcctacgaacgccaccta
ttctcaccgcgcgtaccccccaaacgccaagaaaacggcacatgcgagcccaacccgcgcctcaacttctaccccgtatttgccgtgccagaggt
gcttgccacctatcacatctttttccaaaactgcaagatacccctatcctgccgtgccaaccgcagccgagcggacaagcagctggccttgcggc
agggcgctgtcatacctgatatcgcctcgctcaacgaagtgccaaaaatctttgagggtcttggacgcgacgagaagcgcgcggcaaacgctctg
caacaggaaaacagcgaaaatgaaagtcactctggagtgttggtggaactcgagggtgacaacgcgcgcctagccgtactaaaacgcagcatcga
ggtcacccactttgcctacccggcacttaacctaccccccaaggtcatgagcacagtcatgagtgagctgatcgtgcgccgtgcgcagcccctgg
agagggatgcaaatttgcaagaacaaacagaggagggcctacccgcagttggcgacgagcagctagcgcgctggcttcaaacgcgcgagcctgcc
gacttggaggagcgacgcaaactaatgatggccgcagtgctcgttaccgtggagcttgagtgcatgcagcggttctttgctgacccggagatgca
gcgcaagctagaggaaacattgcactacacctttcgacagggctacgtacgccaggcctgcaagatctccaacgtggagctctgcaacctggtct
cctaccttggaattttgcacgaaaaccgccttgggcaaaacgtgcttcattccacgctcaagggcgaggcgcgccgcgactacgtccgcgactgc
gtttacttatttctatgctacacctggcagacggccatgggcgtttggcagcagtgcttggaggagtgcaacctcaaggagctgcagaaactgct
aaagcaaaacttgaaggacctatggacggccttcaacgagcgctccgtggccgcgcacctggcggacatcattttccccgaacgcctgcttaaaa
ccctgcaacagggtctgccagacttcaccagtcaaagcatgttgcagaactttaggaactttatcctagagcgctcaggaatcttgcccgccacc
tgctgtgcacttcctagcgactttgtgcccattaagtaccgcgaatgccctccgccgctttggggccactgctaccttctgcagctagccaacta
ccttgcctaccactctgacataatggaagacgtgagoggtgacggtctactggagtgtcactgtcgctgcaacctatgcaccccgcaccgctccc
tggtttgcaattcgcagctgcttaacgaaagtcaaattatcggtacctttgagctgcagggtccctcgcctgacgaaaagtccgcggctccgggg
ttgaaactcactccggggctgtggacgtcggcttaccttcgcaaatttgtacctgaggactaccacgcccacgagattaggttctacgaagacca
atcccgcccgcctaatgcggagcttaccgcctgcgtcattacccagggccacattcttggccaattgcaagccatcaacaaagcccgccaagagt
ttctgctacgaaagggacggggggtttacttggacccccagtccggcgaggagctcaacccaatccccccgccgccgcagccctatcagcagcag
ccgcgggcccttgcttcccaggatggcacccaaaaagaagctgcagctgccgccgccacccacggacgaggaggaatactgggacagtcaggcag
aggaggttttggacgaggaggaggaggacatgatggaagactgggagagcctagacgaggaagcttccgaggtcgaagaggtgtcagacgaaaca
ccgtcaccctcggtcgcattcccctcgccggcgccccagaaatcggcaaccggttccagcatggctacaacctccgctcctcaggcgccgccggc
actgcccgttcgccgacccaaccgtagatgggacaccactggaaccagggccggtaagtccaagcagccgccgccgttagcccaagagcaacaac
agcgccaaggctaccgctcatggcgcgggcacaagaacgccatagttgcttgcttgcaagactgtgggggcaacatctccttcgcccgccgcttt
cttctctaccatcacggcgtggccttcccccgtaacatcctgcattactaccgtcatctctacagcccatactgcaccggcggcagcggcagcaa
cagcagcggccacacagaagcaaaggcgaccggatagcaagactctgacaaagcccaagaaatccacagcggcggcagcagcaggaggaggagcg
ctgcgtctggcgcccaacgaacccgtatcgacccgcgagcttagaaacaggatttttcccactctgtatgctatatttcaacagagcaggggcca
agaacaagagctgaaaataaaaaacaggtctctgcgatccctcacccgcagctgcctgtatcacaaaagcgaagatcagcttcggcgcacgctgg
aagacgcggaggctctcttcagtaaatactgcgcgctgactcttaaggactagtttcgcgccctttctcaaatttaagcgcgaaaactacgtcat
ctccagcggccacacccggcgccagcacctgttgtcagcgccattatgagcaaggaaattcccacgccctacatgtggagttaccagccacaaat
gggacttgcggctggagctgcccaagactactcaacccgaataaactacatgagcgcgggaccccacatgatatcccgggtcaacggaatacgcg
cccaccgaaaccgaattctcctggaacaggcggctattaccaccacacctcgtaataaccttaatccccgtagttggcccgctgccctggtgtac
caggaaagtcccgctcccaccactgtggtacttcccagagacgcccaggccgaagttcagatgactaactcaggggcgcagcttgcgggcggctt
tcgtcacagggtgcggtcgcccgggcagggtataactcacctgacaatcagagggcgaggtattcagctcaacgacgagtcggtgagctcctcgc
ttggtctccgtccggacgggacatttcagatcggcggcgccggccgctcttcattcacgcctcgtcaggcaatcctaactctgcagacctcgtcc
tctgagccgcgctctggaggcattggaactctgcaatttattgaggagtttgtgccatcggtctactttaaccccttctcgggacctcccggcca
ctatccggatcaatttattcctaactttgacgcggtaaaggactcggcggacggctacgactgaatgttaagtggagaggcagagcaactgcgcc
tgaaacacctggtccactgtcgccgccacaagtgctttgcccgcgactccggtgagttttgctactttgaattgcccgaggatcatatcgagggc
ccggcgcacggcgtccggcttaccgcccagggagagcttgcccgtagcctgattcgggagtttacccagcgccccctgctagttgagcgggacag
gggaccctgtgttctcactgtgatttgcaactgtcctaaccctggattacatcaagatctttgttgccatctctgtgctgagtataataaataca
gaaattaaaatatactggggctcctatcgccatcctgtaaacgccaccgtcttcacccgcccaagcaaaccaaggcgaaccttacctggtacttt
taacatctctccctctgtgatttacaacagtttcaacccagacggagtgagtctacgagagaacctctccgagctcagctactccatcagaaaaa
acaccaccctccttacctgccgggaacgtacgagtgcgtcaccggccgctgcaccacacctaccgcctgaccgtaaaccagactttttccggaca
gacctcaataactctgtttaccagaacaggaggtgagcttagaaaacccttagggtattaggccaaaggcgcagctactgtggggtttatgaaca
attcaagcaactctacgggctattctaattcaggtttctctagaatcggggttggggttattctctgtcttgtgattctctttattcttatacta
acgcttctctgcctaaggctcgccgcctgctgtgtgcacatttgcatttattgtcagctttttaaacgctggggtcgccacccaagatgattagg
tacataatcctaggtttactcacccttgcgtcagcccacggtaccacccaaaaggtggattttaaggagccagcctgtaatgttacattcgcagc
tgaagctaatgagtgcaccactcttataaaatgcaccacagaacatgaaaagctgcttattcgccacaaaaacaaaattggcaagtatgctgttt
atgctatttggcagccaggtgacactacagagtataatgttacagttttccagggtaaaagtcataaaacttttatgtatacttttccattttat
gaaatgtgcgacattaccatgtacatgagcaaacagtataagttgtggcccccacaaaattgtgtggaaaacactggcactttctgctgcactgc
tatgctaattacagtgctcgctttggtctgtaccctactctatattaaatacaaaagcagacgcagctttattgaggaaaagaaaatgccttaat
ttactaagttacaaagctaatgtcaccactaactgctttactcgctgcttgcaaaacaaattcaaaaagttagcattataattagaataggattt
aaaccccccggtcatttcctgctcaataccattcccctgaacaattgactctatgtgggatatgctccagcgctacaaccttgaagtcaggcttc
ctggatgtcagcatctgactttggccagcacctgtcccgcggatttgttccagtccaactacagcgacccaccctaacagagatgaccaacacaa
ccaacgcggccgccgctaccggacttacatctaccacaaatacaccccaagtttctgcctttgtcaataactgggataacttgggcatgtggtgg
ttctccatagcgcttatgtttgtatgccttattattatgtggctcatctgctgcctaaagcgcaaacgcgcccgaccacccatctatagtcccat
cattgtgctacacccaaacaatgatggaatccatagattggacggactgaaacacatgttcttttctcttacagtatgattaaatgagacatgat
tcctcgagtttttatattactgacccttgttgcgcttttttgtgcgtgctccacattggctgcggtttctcacatcgaagtagactgcattccag
ccttcacagtctatttgctttacggatttgtcaccctcacgctcatctgcagcctcatcactgtggtcatcgcctttatccagtgcattgactgg
gtctgtgtgcgctttgcatatctcagacaccatccccagtacagggacaggactatagctgagcttcttagaattctttaattatgaaatttact
gtgacttttctgctgattatttgcaccctatctgcgttttgttccccgacctccaagcctcaaagacatatatcatgcagattcactcgtatatg
gaatattccaagttgctacaatgaaaaaagcgatctttccgaagcctggttatatgcaatcatctctgttatggtgttctgcagtaccatcttag
ccctagctatatatccctaccttgacattggctggaacgcaatagatgccatgaaccacccaactttccccgcgcccgctatgcttccactgcaa
caagttgttgccggcggctttgtcccagccaatcagcctcgcccaccttctcccacccccactgaaatcagctactttaatctaacaggaggaga
tgactgacaccctagatctagaaatggacggaattattacagagcagcgcctgctagaaagacgcagggcagcggccgagcaacagcgcatgaat
caagagctccaagacatggttaacttgcaccagtgcaaaaggggtatcttttgtctcgtaaagcaggccaaagtcacctacgacagtaataccac
cggacaccgccttagctacaagttgccaaccaagcgtcagaaattggtggtcatggtgggagaaaagcccattaccataactcagcactcggtag
aaaccgaaggctgcattcactcaccttgtcaaggacctgaggatctctgcacccttattaagaccctgtgcggtctcaaagatcttattcccttt
aactaataaaaaaaaataataaagcatcacttacttaaaatcagttagcaaatttctgtccagtttattcagcagcacctccttgccctcctccc
agctctggtattgcagcttcctcctggctgcaaactttctccacaatctaaatggaatgtcagtttcctcctgttcctgtccatccgcacccact
atcttcatgttgttgcagatgaagcgcgcaagaccgtctgaagataccttcaaccccgtgtatccatatgacacggaaaccggtcctccaactgt
gccttttcttactcctccctttgtatcccccaatgggtttcaagagagtccccctggggtactctctttgcgcctatccgaacctctagttacct
ccaatggcatgcttgcgctcaaaatgggcaacggcctctctctggacgaggccggcaaccttacctcccaaaatgtaaccactgtgagcccacct
ctccgaggagacaagtcaaacataaacctggaaatatctgcacccctcacagttacctcagaagccctaactgtggctgccgccgcacctctaat
ggtcgcgggcaacacactcaccatgcaatcacaggccccgctaaccgtgcacgactccaaacttagcattgccacccaaggacccctcacagtgt
cagaaggaaagctagccctgcaaacatcaggccccctcaccaccaccgatagcagtacccttactatcactgcctcaccccctctaactactgcc
actggtagcttgggcattgacttgaaagagcccatttatacacaaaatggaaaactaggactaaagtacggggctcctttgcatgtaacagacga
cctaaacactttgaccgtagcaactggtccaggtgtgactattaataatacttccttgcaaactaaagttactggagccttgggttttgattcac
aaggcaatatgcaacttaatgtagcaggaggactaaggattgattctcaaaacagacgccttatacttgatgttagttatccgtttgatgctcaa
aaccaactaaatctaagactaggacagggccctctttttataaactcagcccacaacttggatattaactacaacaaaggcctttacttgtttac
agcttcaaacaattccaaaaagcttgaggttaacctaagcactgccaaggggttgatgtttgacgctacagccatagccattaatgcaggagatg
ggcttgaatttggttcacctaatgcaccaaacacaaatcccctcaaaacaaaaattggccatggcctagaatttgattcaaacaaggctatggtt
cctaaactaggaactggccttagttttgacagcacaggtgccattacagtaggaaacaaaaataatgataagctaactttgtggaccacaccagc
tccatctcctaactgtagactaaatgcagagaaagatgctaaactcactttggtcttaacaaaatgtggcagtcaaatacttgctacagtttcag
ttttggctgttaaaggcagtttggctccaatatctggaacagttcaaagtgctcatcttattataagatttgacgaaaatggagtgctactaaac
aattccttcctggacccagaatattggaactttagaaatggagatcttactgaaggcacagcctatacaaacgctgttggatttatgcctaacct
atcagcttatccaaaatctcacggtaaaactgccaaaagtaacattgtcagtcaagtttacttaaacggagacaaaactaaacctgtaacactaa
ccattacactaaacggtacacaggaaacaggagacacaactccaagtgcatactctatgtcattttcatgggactggtctggccacaactacatt
aatgaaatatttgccacatcctcttacactttttcatacattgcccaagaataaagaatcgtttgtgttatgtttcaacgtgtttatttttcaat
tgtactaagcggtgatgtttctgatcagccaccatgggagtgctaaaattcaagcacatctttttcagaagctttgttaaatcaagtggagtatc
ccagatagttttcaccttccttctgattccatgttgcttgactctgaatttcagagcacctcctgttattccaaatgtgcctttcctctgggcct
ggaatgccccaagtgaattttgtcttggaaaatttgatgagccactagatatgagcctcttctctttcataggaagcccccgaataaacgccacc
gggcaaggtgttacaatattttatgttgatagacttggctactatccttacatagattcaatcacaggagtaactgtgaatggaggaatccccca
gaagatttccttacaagaccatctggacaaagctaagaaagacattacattttatatgccagtagacaatttgggaatggctgttattgactggg
aagaatggagacccacttgggcaagaaactggaaacctaaagatgtttacaagaataggtctattgaattggttcagcaacaaaatgtacaactt
agtctcacagaggccactgagaaagcaaaacaagaatttgaaaaggcagggaaggatttcctggtagagactataaaattgggaaaattacttcg
gccaaatcacttgtggggttattatctttttccggattgttacaaccatcactataagaaacccggttacaatggaagttgcttcaatgtagaaa
taaaaagaaatgatgatctcagctggttgtggaatgaaagcactgctctttacccatccatttatttgaacactcagcagtctcctgtagctgct
acactctatgtgcgcaatcgagttcgggaagccatcagagtttccaaaatacctgatgcaaaaagtccacttccggtttttgcatatacccgcat
agtttttactgatcaagttttgaaattcctttctcaagatgaacttgtgtatacatttggcgaaactgttgctctgggtgcttctggaattgtaa
tatggggaaccctcagtataatgcgaagtatgaaatcttgcttgctcctagacaattacatggagactatactgaatccttacataatcaacgtc
acactagcagccaaaatgtgtagccaagtgctttgccaggagcaaggagtgtgtataaggaaaaactggaattcaagtgactatcttcacctcaa
cccagataattttgctattcaacttgagaaaggtggaaagttcacagtacgtggaaaaccgacacttgaagacctggagcaattttctgaaaaat
tttattgcagctgttatagcaccttgagttgtaaggagaaagctgatgtaaaagacactgatgctgttgatgtgtgtattgctgatggtgtctgt
atagatgcttttctaaaacctcccatggagacagaagaacctcaaattttctacaatgcttcaccctccacactatcttaataaactttattttt
caattgcagaaaatttcaagtcatttttcattcagtagtatagccccaccaccacatagcttatacagatcaccgtaccttaatcaaactcacag
aaccctagtattcaacctgccacctccctcccaacacacagagtacacagtcctttctccccggctggccttaaaaagcatcatatcatgggtaa
cagacatattcttaggtgttatattccacacggtttcctgtcgagccaaacgctcatcagtgatattaataaactccccgggcagctcacttaag
ttcatgtcgctgtccagctgctgagccacaggctgctgtccaacttgcggttgcttaacgggcggcgaaggagaagtccacgcctacatgggggt
agagtcataatcgtgcatcaggatagggcggtggtgctgcagcagcgcgcgaataaactgctgccgccgccgctccgtcctgcaggaatacaaca
tggcagtggtctcctcagcgatgattcgcaccgcccgcagcataaggcgccttgtcctccgggcacagcagcgcaccctgatctcacttaaatca
gcacagtaactgcagcacagcaccacaatattgttcaaaatcccacagtgcaaggcgctgtatccaaagctcatggggggaccacagaacccacg
tggccatcataccacaagcgcaggtagattaagtggcgacccctcataaacacgctggacataaacattacctcttttggcatgttgtaattcac
cacctcccggtaccatataaacctctgattaaacatggcgccatccaccaccatcctaaaccagctggccaaaacctgcccgccggctatacact
gcagggaaccgggactggaacaatgacagtggagagcccaggactcgtaaccatggatcatcatgctcgtcatgatatcaatgttggcacaacac
aggcacacgtgcatacacttcctcaggattacaagctcctcccgcgttagaaccatatcccagggaacaacccattcctgaatcagcgtaaatcc
cacactgcagggaagacctcgcacgtaactcacgttgtgcattgtcaaagtgttacattcgggcagcagcggatgatcctccagtatggtagcgc
gggtttctgtctcaaaaggaggtagacgatccctactgtacggagtgcgccgagacaaccgagatcgtgttggtcgtagtgtcatgccaaatgga
acgccggacgtagtcatatttcctgaagcaaaaccaggtgcgggcgtgacaaacagatctgcgtctccggtctcgccgcttagatcgctctgtgt
agtagttgtagtatatccactctctcaaagcatccaggcgccccctggcttcgggttctatgtaaactccttcatgcgccgctgccctgataaca
tccaccaccgcagaataagccacacccagccaacctacacattcgttctgcgagtcacacacgggaggagcgggaagagctggaagaaccatgtt
tttttttttattccaaaagattatccaaaacctcaaaatgaagatctattaagtgaacgcgctcccctccggtggcgtggtcaaactctacagcc
aaagaacagataatggcatttgtaagatgttgcacaatggcttccaaaaggcaaacggccctcacgtccaagtggacgtaaaggctaaacccttc
agggtgaatctcctctataaacattccagcaccttcaaccatgcccaaataattctcatctcgccaccttctcaatatatctctaagcaaatccc
gaatattaagtccggccattgtaaaaatctgctccagagcgccctccaccttcagcctcaagcagcgaatcatgattgcaaaaattcaggttcct
cacagacctgtataagattcaaaagcggaacattaacaaaaataccgcgatcccgtaggtcccttcgcagggccagctgaacataatcgtgcagg
tctgcacggaccagcgcggccacttccccgccaggaaccatgacaaaagaacccacactgattatgacacgcatactcggagctatgctaaccag
cgtagccccgatgtaagcttgttgcatgggcggcgatataaaatgcaaggtgctgctcaaaaaatcaggcaaagcctcgcgcaaaaaagaaagca
catcgtagtcatgctcatgcagataaaggcaggtaagctccggaaccaccacagaaaaagacaccatttttctctcaaacatgtctgcgggtttc
tgcataaacacaaaataaaataacaaaaaaacatttaaacattagaagcctgtcttacaacaggaaaaacaacccttataagcataagacggact
acggccatgccggcgtgaccgtaaaaaaactggtcaccgtgattaaaaagcaccaccgacagctcctcggtcatgtccggagtcataatgtaaga
ctcggtaaacacatcaggttgattcacatcggtcagtgctaaaaagcgaccgaaatagcccgggggaatacatacccgcaggcgtagagacaaca
ttacagcccccataggaggtataacaaaattaataggagagaaaaacacataaacacctgaaaaaccctcctgcctaggcaaaatagcaccctcc
cgctccagaacaacatacagcgcttccacagoggcagccataacagtcagccttaccagtaaaaaagaaaacctattaaaaaaacaccactcgac
acggcaccagctcaatcagtcacagtgtaaaaaagggccaagtgcagagcgagtatatataggactaaaaaatgacgtaacggttaaagtccaca
aaaaacacccagaaaaccgcacgcgaacctacgcccagaaacgaaagccaaaaaacccacaacttcctcaaatcgtcacttccgttttcccacgt
tacgtcacttcccattttaagaaaactacaattcccaacacatacaagttactccgccctaaaacctacgtcacccgccccgttcccacgccccg
cgccacgtcacaaactccaccccctcattatcatattggcttcaatccaaaataaggtatattattgatgatg
SEQ ID NO: 4
Amino acid sequence of the modified version of the fiber of adenovirus serotype in which RGDK
modification has been introduced
MKRARPSEDTFNPVYPYDTETGPPTVPFLTPPFVSPNGFQESPPGVLSLRLSEPLVTSNGMLALKMGNGLSLDEAGNLTSQNVTTVSPPLRGDKS
NINLEISAPLTVTSEALTVAAAAPLMVAGNTLTMGSQAPLTVHDSKLSIATQGPLTVSEGKLALQTSGPLTTTDSSTLTITASPPLTTATGSLGI
DLKEPIYTQNGKLGLKYGAPLHVTDDLNTLTVATGPGVTINNTSLGTKVTGALGFDSQGNMQLNVAGGLRIDSQNRRLILDVSYPFDAQNQLNLR
LGQGPLFINSAHNLDINYNKGLYLFTASNNSKKLEVNLSTAKGLMFDATAIAINAGDGLEFGSPNAPNTNPLKTKIIGHGLEFDSNKAMVPKLGT
GLSFDSTGAITVGNKNNDKLTLWTTPAPSPNCDLNAEKDAKLTLVLTKCGSQILATVSVLAVKGSLAPISGTVQSAHLIIRFDENGVLLNNSFLD
PEYWNFRNGDLTEGTAYTNAVGFMPNLSAYPKSHGKTAKSNIVSQVYLNGDKTKPVTLTITLNGTQETGDTTPSAYSMSFSWDWSGHNYINEIFA
TSSYTF
SEQ ID NO: 5
RGD-C peptide
CDCRGDCFC
SEQ ID NO: 6
binding domain
KKTK
SEQ ID NO: 7
binding domain
RGDK
SEQ ID NO: 8
Translation of SEQ ID NO: 1 (PH20 with carboxy-terminal domain present)
QIVFTFLLIPCCLTLNFRAPPVIPNVPFLWAWNAPSEFCLGKFDEPLDMSLFSFIGMGVLKFKHIFFRSFVKSSGVSSPRINATGQGVTIFYVDR
LGYYPYIDSITGVTVNGGIPQKISLQDHLDKAKKDITFYMPVDNLGMAVIDWEEWRPTWARNWKPKDVYKNRSIELVQQQNVQLSLTEATEKAKQ
EFEKAGKDFLVETIKLGKLLRPNHLWGYYLFPDCYNHHYKKPGYNGSCFNVEIKRNDDLSWLWNESTALYPSIYLNTQQSPVAATLYVRNRVREA
IRVSKIPDAKSPLPVFAYTRIVFTDQVLKFLSQDELVYTFGETVALGASGIVIWGTLSIMRSMKSCLLLDNYMETILNPYIINVTLAAKMCSQVL
CQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMET
EEPQIFYNASPSTLSATMFIVSILFLIISSVASL-
SEQ ID NO: 9
cDNA encoding PH20 with carboxy-terminal domain deleted
atgggagtgctaaaattcaagcacatctttttcagaagctttgttaaatcaagtggagtatcccagatagttttcaccttccttctgattccatg
ttgcttgactctgaatttcagagcacctcctgttattccaaatgtgcctttcctctgggcctggaatgccccaagtgaattttgtcttggaaaat
ttgatgagccactagatatgagcctcttctctttcataggaagcccccgaataaacgccaccgggcaaggtgttacaatattttatgttgataga
cttggctactatccttacatagattcaatcacaggagtaactgtgaatggaggaatcccccagaagatttccttacaagaccatctggacaaagc
taagaaagacattacattttatatgccagtagacaatttgggaatggctgttattgactgggaagaatggagacccacttgggcaagaaactgga
aacctaaagatgtttacaagaataggtctattgaattggttcagcaacaaaatgtacaacttagtctcacagaggccactgagaaagcaaaacaa
gaatttgaaaaggcagggaaggatttcctggtagagactataaaattgggaaaattacttcggccaaatcacttgtggggttattatctttttcc
ggattgttacaaccatcactataagaaacccggttacaatggaagttgcttcaatgtagaaataaaaagaaatgatgatctcagctggttgtgga
atgaaagcactgctctttacccatccatttatttgaacactcagcagtctcctgtagctgctacactctatgtgcgcaatcgagttcgggaagcc
atcagagtttccaaaatacctgatgcaaaaagtccacttccggtttttgcatatacccgcatagtttttactgatcaagttttgaaattcctttc
tcaagatgaacttgtgtatacatttggcgaaactgttgctctgggtgcttctggaattgtaatatggggaaccctcagtataatgcgaagtatga
aatcttgcttgctcctagacaattacatggagactatactgaatccttacataatcaacgtcacactagcagccaaaatgtgtagccaagtgctt
tgccaggagcaaggagtgtgtataaggaaaaactggaattcaagtgactatcttcacctcaacccagataattttgctattcaacttgagaaagg
tggaaagttcacagtacgtggaaaaccgacacttgaagacctggagcaattttctgaaaaattttattgcagctgttatagcaccttgagttgta
aggagaaagctgatgtaaaagacactgatgctgttgatgtgtgtattgctgatggtgtctgtatagatgcttttctaaaacctcccatggagaca
gaagaacctcaaattttctacaatgcttcaccctccacactatottaa
SEQ ID NO: 10
Translation of SEQ ID NO: 9 (PH20 with carboxy-terminal domain deleted)
MGVLKFKHIFFRSFVKSSGVSQIVFTFLLIPCCLTLNFRAPPVIPNVPFLWAWNAPSEFCLGKFDEPLDMSLFSFIGSPRINATGQGVTIFYVDR
LGYYPYIDSITGVTVNGGIPQKISLQDHLDKAKKDITFYMPVDNLGMAVIDWEEWRPTWARNWKPKDVYKNRSIELVQQQNVQLSLTEATEKAKQ
EFEKAGKDFLVETIKLGKLLRPNHLWGYYLFPDCYNHHYKKPGYNGSCFNVEIKRNDDLSWLWNESTALYPSIYLNTQQSPVAATLYVRNRVREA
IRVSKIPDAKSPLPVFAYTRIVFTDQVLKFLSQDELVYTFGETVALGASGIVIWGTLSIMRSMKSCLLLDNYMETILNPYIINVTLAAKMCSQVL
CQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMET
EEPQIFYNASPSTLS-

What is claimed is:

1. A method for treating retinoblastoma, Ewing sarcoma, or neuroblastoma in a patient in need thereof, comprising co-administering to the patient (i) an oncolytic adenovirus comprising replication machinery specific for tumor cells and, optionally, a polynucleotide sequence encoding a hyaluronidase enzyme inserted into its genome and (ii) a topoisomerase I inhibitor or a prodrug thereof.

2. The method according to claim 1, wherein the oncolytic adenovirus is administered by intraocular, intravenous, intraarterial, intrathecal, intravitreal, or intratumoral injection.

3. The method according to claim 1 or 2, wherein the topoisomerase I inhibitor or prodrug thereof is administered intravenously, intraarterially, intravitreally, intrathecally, intraventricularly, or orally.

4. The method according to any one of claims 1-3, wherein the oncolytic adenovirus and the topoisomerase I inhibitor or prodrug thereof are formulated in separate compositions.

5. The method according to any one of claims 1-3, wherein the oncolytic adenovirus and the topoisomerase I inhibitor or prodrug thereof are formulated in a single composition.

6. The method according to any one of claims 1-5, wherein the separate compositions are administered simultaneously or contemporaneously.

7. The method according to any one of claims 1-6, wherein the oncolytic adenovirus is administered first and the topoisomerase I inhibitor or prodrug thereof is administered within about 60 minutes of the administration of the oncolytic adenovirus.

8. The method of claim 7, wherein the topoisomerase I inhibitor or prodrug thereof is administered within about 30 minutes, within about 20 minutes, within about 10 minutes, within about 5 minutes, or within about 1 minute of the administration of the oncolytic adenovirus.

9. The method according to any one of claims 1-8, wherein the subject is administered one intrathecal injection of the oncolytic adenovirus concomitant with systemic or intrathecal cycles of the topoisomerase I inhibitor or prodrug thereof.

10. The method according to any one of claims 1-8, wherein the subject is administered the oncolytic adenovirus and the topoisomerase I inhibitor or prodrug thereof systemically.

11. The method according to any one of claims 1-10, wherein the oncolytic adenovirus and/or topoisomerase I inhibitor or prodrug thereof is co-administered with one or more local and/or systemic corticosteroids, optionally wherein the one or more local and/or systemic corticosteroids are selected from hydrocortisone, prednisone, prednisolone, methylprednisolone, dexamethasone, betamethasone, alsosterone, budesonide, fluticasone, flunisolide, ciclesonide, mometasone, beclomethasone, triamcinolone, and tixocortol.

12. The method according to any one of claims 1-11, wherein the topoisomerase I inhibitor is topotecan or SN-38 and/or the topoisomerase I inhibitor prodrug is irinotecan.

13. The method according to any one of claims 1-12, wherein the hyaluronidase enzyme is the human hyaluronidase enzyme PH20.

14. The method according to claim 13, wherein the sequence that encodes a hyaluronidase enzyme is SEQ ID NO: 9, or a sequence having at least about 75%, at least about 80%, at least about 85%, at least about 90%, at least about 95%, at least about 98%, or at least about 99% sequence identity thereto.

15. The method according to any one of claims 1-14, wherein the oncolytic adenovirus is generated from a human adenovirus serotype 5.

16. The method according to any one of claims 1-15, wherein the treatment is of retinoblastoma and the oncolytic adenoviral replication occurs in tumor cells with an aberrant Rb-E2F pathway and not in healthy, non-tumor, or normal cells.

17. The method according to claim 16, wherein the oncolytic adenovirus is engineered to replicate in tumor cells and not healthy, non-tumor, or normal cells by deletion of a Rb binding domain or deletion Δ24 in the sequence coding for the E1a protein and the insertion of four binding sites to E2F-1 and one binding site to Sp1 into the endogenous promoter of E1a to control the expression of E1a.

18. The method according to any one of claims 1-17, wherein the oncolytic adenovirus has the capsid modified such that the heparan sulfate binding domain ⁹¹KKTK⁹⁴ (SEQ ID NO: 8) present in the adenovirus fiber has been replaced, optionally by the domain ⁹¹RGDK⁹⁴ (SEQ ID NO: 9).

19. The method according to any one of claims 1-18, wherein the oncolytic adenovirus is VCN-01 (SEQ ID NO: 3).

20. The method according to any one of claims 1-19, wherein the patient is a human patient.

21. The method according to claim 20, wherein the human patient is a pediatric human patient.

22. The method according to any one of claims 1-21, wherein the retinoblastoma, Ewing sarcoma, or neuroblastoma is a retinoblastoma, Ewing sarcoma, or neuroblastoma resistant to conventional chemotherapy and/or radiotherapy treatment.

23. The method according to any one of claims 1-22, wherein the method improves and/or increases and/or enhances antitumor efficacy compared to treatment with the topoisomerase I inhibitor or prodrug thereof without the oncolytic adenovirus.

24. The method according to any one of claims 1-23, wherein the method results in reduced or maintained tumor size, and/or prevention or reduction of metastasis, secondary malignancy, or trilateral retinoblastoma associated with retinoblastoma, compared to treatment with the topoisomerase I inhibitor or prodrug thereof without the oncolytic adenovirus.

25. The method according to any one of claims 1-23, wherein the method results in reduced or maintained tumor size, and/or prevention or reduction of metastasis, secondary malignancy, or recurrent disease associated with Ewing sarcoma, compared to compared to treatment with the topoisomerase I inhibitor or prodrug thereof without the oncolytic adenovirus.

26. The method according to any one of claims 1-23, wherein the method results in reduced or maintained tumor size, and/or prevention or reduction of metastasis, secondary malignancy, or recurrent disease associated with neuroblastoma, compared to compared to treatment with the topoisomerase I inhibitor or prodrug thereof without the oncolytic adenovirus.

27. The method according to any one of claims 1-26, wherein administration of the oncolytic adenovirus reduces apoptotic action of topotecan as compared to monotherapy.

28. The method according to any one of claims 1-27, wherein administration of topotecan following administration of the oncolytic adenovirus leads to S-phase cell cycle arrest.

29. The method according to claim 28, wherein the S-phase cell cycle arrest results in increased infectivity of the oncolytic adenovirus as compared to treatment with the oncolytic adenovirus without the topoisomerase I inhibitor or prodrug thereof.

30. The method according to any one of claims 1-29, wherein administration of topotecan following administration of the oncolytic adenovirus leads to increased E2F-1, p21 and/or cyclin E1 expression as compared to treatment with the oncolytic adenovirus without the topoisomerase I inhibitor or prodrug thereof.

31. The method according to claim 30, wherein the increased E2F-1 expression results in increased oncolytic activity of the oncolytic adenovirus as compared to treatment with the oncolytic adenovirus without the topoisomerase I inhibitor or prodrug thereof.

32. The method according to any one of claims 1-31, wherein the increased infectivity and oncolytic activity of the oncolytic adenovirus occurs without substantially increasing replication of the oncolytic adenovirus.

33. The method according to any one of claims 1-32, wherein sequential treatments in which the oncolytic adenovirus is administered first, followed by systemic topotecan, enhance or increase cell infection and/or anticancer efficacy of the oncolytic adenovirus.

34. A method for treating retinoblastoma, Ewing sarcoma, or neuroblastoma in a patient in need thereof, comprising administering to the patient (i) an oncolytic adenovirus comprising replication machinery specific for tumor cells and, optionally, a polynucleotide sequence encoding a hyaluronidase enzyme inserted into its genome and (ii) a topoisomerase I inhibitor or a prodrug thereof.

35. The method according to claim 34, wherein the oncolytic adenovirus is administered by intraocular, intravenous, intraarterial, intrathecal, intravitreal, or intratumoral injection.

36. The method according to claim 34 or 35, wherein the topoisomerase I inhibitor or prodrug thereof is administered intravenously, intraarterially, intravitreally, intrathecally, intraventricularly, or orally.

37. The method according to any one of claims 34-36, wherein the oncolytic adenovirus and the topoisomerase I inhibitor or prodrug thereof are formulated in separate compositions.

38. The method according to any one of claims 34-37, wherein the oncolytic adenovirus and the topoisomerase I inhibitor or prodrug thereof are formulated in a single composition.

39. The method according to any one of claims 34-38, wherein the oncolytic adenovirus is administered first and the topoisomerase I inhibitor or prodrug thereof is administered within about 12 weeks of the administration of the oncolytic adenovirus.

40. The method of claim 39, wherein the topoisomerase I inhibitor or prodrug thereof is administered within about 60 minutes, about 2 hours, about 3 hours, about 4 hours, about 5 hours, about 6 hours, about 7 hours, about 8 hours, about 9 hours, about 10 hours, about 11 hours, about 12 hours, about 1 day, about 2 days, about 3 days, about 4 days, about 5 days, about 6 days, about 7 days, about 8 days, about 9 days, about 10 days, about 11 days, about 12 days, about 13 days, about 2 weeks, about 3 weeks, about 4 weeks, about 5 weeks, about 6 weeks, about 7 weeks, about 8 weeks, about 9 weeks, about 10 weeks, or about 11 weeks of the administration of the oncolytic adenovirus.

41. The method according to any one of claims 34-40, wherein the subject is administered one intrathecal injection of the oncolytic adenovirus concomitant with systemic or intrathecal cycles of the topoisomerase I inhibitor or prodrug thereof.

42. The method according to any one of claims 34-40, wherein the subject is administered the oncolytic adenovirus and the topoisomerase I inhibitor or prodrug thereof systemically.

43. The method according to any one of claims 34-42, wherein the oncolytic adenovirus and/or topoisomerase I inhibitor or prodrug thereof is co-administered with one or more local and/or systemic corticosteroids, optionally wherein the one or more local and/or systemic corticosteroids are selected from hydrocortisone, prednisone, prednisolone, methylprednisolone, dexamethasone, betamethasone, alsosterone, budesonide, fluticasone, flunisolide, ciclesonide, mometasone, beclomethasone, triamcinolone, and tixocortol.

44. The method according to any one of claims 34-43, wherein the topoisomerase I inhibitor is topotecan or SN-38 and/or the prodrug of the topoisomerase I inhibitor is irinotecan.

45. The method according to any one of claims 34-44, wherein the hyaluronidase enzyme is the human hyaluronidase enzyme PH20.

46. The method according to claim 45, wherein the sequence that encodes a hyaluronidase enzyme is SEQ ID NO: 9, or a sequence having at least about 75%, at least about 80%, at least about 85%, at least about 90%, at least about 95%, at least about 98%, or at least about 99% sequence identity thereto.

47. The method according to any one of claims 34-46, wherein the oncolytic adenovirus is generated from a human adenovirus serotype 5.

48. The method according to any one of claims 34-47, wherein the oncolytic adenoviral replication occurs in tumor cells with an aberrant Rb-E2F pathway and not in healthy, non-tumor, or normal cells.

49. The method according to claim 48, wherein the oncolytic adenovirus is engineered to replicate in tumor cells and not healthy, non-tumor, or normal cells by deletion Δ24 in the sequence coding for the E1a protein and the insertion of four binding sites to E2F-1 and one binding site to Sp1 into the endogenous promoter of E1a to control the expression of E1a.

50. The method according to any one of claims 34-49, wherein the oncolytic adenovirus has the capsid modified such that the heparan sulfate binding domain ⁹¹KKTK⁹⁴ (SEQ ID NO: 8) present in the adenovirus fiber has been replaced, optionally by the domain ⁹¹RGDK⁹⁴ (SEQ ID NO: 9).

51. The method according to any one of claims 34-50, wherein the oncolytic adenovirus is VCN-01 (SEQ ID NO: 3).

52. The method according to any one of claims 34-51, wherein the patient is a human patient.

53. The method according to claim 52, wherein the human patient is a pediatric human patient.

54. The method according to any one of claims 34-53, wherein the retinoblastoma, Ewing sarcoma, or neuroblastoma is a retinoblastoma, Ewing sarcoma, or neuroblastoma resistant to conventional chemotherapy and/or radiotherapy treatment.

55. The method according to any one of claims 34-54, wherein the method improves and/or increases and/or enhances antitumor efficacy compared to treatment with the topoisomerase I inhibitor or prodrug thereof without the oncolytic adenovirus.

56. The method according to any one of claims 34-55, wherein the method results in reduced or maintained tumor size, and/or prevention or reduction of metastasis, secondary malignancy, or trilateral retinoblastoma associated with retinoblastoma, compared to treatment with the topoisomerase I inhibitor or prodrug thereof without the oncolytic adenovirus.

57. The method according to any one of claims 34-55, wherein the method results in reduced or maintained tumor size, and/or prevention or reduction of metastasis, secondary malignancy, or recurrent disease associated with Ewing sarcoma, compared to treatment with the topoisomerase I inhibitor or prodrug thereof without the oncolytic adenovirus.

58. The method according to any one of claims 34-55, wherein the method results in reduced or maintained tumor size, and/or prevention or reduction of metastasis, secondary malignancy, or recurrent disease associated with neuroblastoma, compared to compared to treatment with the topoisomerase I inhibitor or prodrug thereof without the oncolytic adenovirus.

59. The method according to any one of claims 34-58, wherein administration of the oncolytic adenovirus reduces apoptotic action of topotecan as compared to monotherapy.

60. The method according to any one of claims 34-59, wherein administration of topotecan following administration of the oncolytic adenovirus leads to S-phase cell cycle arrest.

61. The method according to claim 60, wherein the S-phase cell cycle arrest results in increased infectivity of the oncolytic adenovirus as compared to treatment with the oncolytic adenovirus without the topoisomerase I inhibitor or prodrug thereof.

62. The method according to any one of claims 34-61, wherein administration of topotecan following administration of the oncolytic adenovirus leads to increased E2F-1, p21 and/or cyclin E1 expression as compared to treatment with the oncolytic adenovirus without the topoisomerase I inhibitor or prodrug thereof.

63. The method according to claim 62, wherein the increased E2F-1 expression results in increased oncolytic activity of the oncolytic adenovirus as compared to treatment with the oncolytic adenovirus without the topoisomerase I inhibitor or prodrug thereof.

64. The method according to any one of claims 34-63, wherein the increased infectivity and oncolytic activity of the oncolytic adenovirus occurs without substantially increasing replication of the oncolytic adenovirus.

65. The method according to any one of claims 34-64, wherein sequential treatments in which the oncolytic adenovirus is administered first, followed by systemic topotecan, enhance or increase cell infection and/or anticancer efficacy of the oncolytic adenovirus.
